SEQUENCE LISTING (32 sequences)

SEQ ID NO: 1 - DNA, 21 bp, Artificial
  siRNA targeted to GFP (sense strand)
ggcuacguccaggagcgcatt

SEQ ID NO: 2 - DNA, 21 bp, Artificial
  siRNA targeted to GFP (antisense strand)
ugcgcuccuggacguagcctt

SEQ ID NO: 3 - DNA, 21 bp, Artificial
  siRNA targeted to influenza virus NP transcript (sense strand)
ggaucuuauuucuucggagtt

SEQ ID NO: 4 - DNA, 21 bp, Artificial
  siRNA targeted to influenza virus NP transcript (antisense strand)
cuccgaagaaauaagaucctt

SEQ ID NO: 5 - DNA, 21 bp, Artificial
  siRNA targeted to influenza virus PA transcript (sense strand)
gcaauugaggagugccugatt

SEQ ID NO: 6 - DNA, 21 bp, Artificial
  siRNA targeted to influenza virus PA transcript (antisense strand)
ucaggcacuccucaauugctt

SEQ ID NO: 7 - DNA, 21 bp, Artificial
  siRNA targeted to infuenza virus B-1 transcript (sense strand)
gaucuguuccaccauugaatt

SEQ ID NO: 8 - DNA, 21 bp, Artificial
  siRNA targeted to influenza virus PB-1 transcript (antisense strand)
uucaaugguggaacagauctt

SEQ ID NO: 9 - DNA, 21 bp, Artificial
  siRNA targeted to influenza virus NP transcript (sense strand)
uagagagaauggugcucuctt

SEQ ID NO: 10 - DNA, 21 bp, Artificial
  siRNA targeted to influenza virus NP transcript (antisense strand)
gagagcaccauucucucuatt

SEQ ID NO: 11 - DNA, 21 bp, siRNA targeted to influenza virus M transcript (sense strand)
ccgaggucgaaacguacgutt

SEQ ID NO: 12 - DNA, 21 bp, Artificial
  siRNA targeted to influenza virus M transcript (antisense strand)
acguacguuucgaccucggtt

SEQ ID NO: 13 - DNA, 21 bp, Artificial
  siRNA targeted to influenza virus PB-1 transcript (sense strand)
caggauacaccauggauactt

SEQ ID NO: 14 - DNA, 21 bp, Artificial
  siRNA targeted to influenza virus PB-1 transcript (antisense strand)
guauccaugguguauccugtt

SEQ ID NO: 15 - DNA, 20 bp, Artificial
  shRNA targeted to influenza virus NP transcript (sense portion)
ggaucuuauuucuucggaga

SEQ ID NO: 16 - DNA, 20 bp, Artificial
  shRNA targeted to influenza virus NP transcript (antisense portion)
ucuccgaagaaauaagaucc

SEQ ID NO: 17 - DNA, 57 bp, Artificial
  oligonucleotide for construction of shRNA targeted to influenza virus NP transcript.
tggatcttatttcttcggagattcaagagatctccgaagaaataagatccttttttc

SEQ ID NO: 18 - DNA, 61 bp, Artificial
  oligonucleotide for construction of shRNA targeted to infuenza virus NP transcript
tcgagaaaaaaggatcttatttcttcggagatctcttgaatctccgaagaaataagatcca

SEQ ID NO: 19 - DNA, 55 bp, Artificial
  oligonucleotide for construction of shRNA targeted to influenza virus PB1 transcript
tgatctgttccaccattgaattcaagagattcaatggtggaacagatcttttttc

SEQ ID NO: 20 - DNA, 59 bp, Artificial
  oligonucleotide for construction of shRNA targeted to influenza virus PB1 transcript
tcgagaaaaaagatctgttccaccattgaatctcttgaattcaatggtggaacagatca

SEQ ID NO: 21 - DNA, 56 bp, Artificial
  oligonucleotide for construction of shRNA targeted to respiratory syncytial virus transcript
tgcgataatataactgcaagattcaagagatcttgcagttatattatcgttttttc

SEQ ID NO: 22 - DNA, 60 bp, Artificial
  oligonucleotide for construction of shRNA targeted to respiratory syncytial virus transcript
tcgagaaaaaacgataatataactgcaagatctcttgaatcttgcagttatattatcgca

SEQ ID NO: 23 - DNA, 19 bp, Artificial
  siRNA targeted to respiratory syncytial virus transcript (sense strand)
cgataatataactgcaaga

SEQ ID NO: 24 - DNA, 19 bp, Artificial
  siRNA targeted to respiratory syncytial virus transcript (antisense strand)
tcttgcagttatattatcg

SEQ ID NO: 25 - DNA, 55 bp, Artificial
  oligonucleotide for construction of shRNA targeted to influenza virus PA transcript
tgcaattgaggagtgcctgattcaagagatcaggcactcctcaattgcttttttc

SEQ ID NO: 26 - DNA, 59 bp, Artificial
  oligonucleotide for construction of shRNA targeted to influenza virus PA transcript
tcgagaaaaaagcaattgaggagtgcctgatctcttgaatcaggcactcctcaattgca

SEQ ID NO: 27 - DNA, 9 bp, Artificial
  loop sequence for shRNA
uucaagaga

SEQ ID NO: 28 - DNA, 60 bp, Artificial
  shRNA targeted to influenza virus NP transcript
cgggguggaucuuauuucuucggaggcagguccacuccgaagaaauaagauccuucccug

SEQ ID NO: 29 - DNA, 60 bp, Artificial
  shRNA targeted to GFP transcript
cgggguugcgcuccuggacguagccgcagguccaggcuacguccaggagcgcauucccug

SEQ ID NO: 30 - DNA, 119 bp, Artificial
  hairpin RNA targeted to GFP and influenza virus NP transcripts
cgggguggaucuuauuucuucggaggcagcgggguugcgcuccuggacguagccgcagguccaggcuacguccaggagcgcauucccuguccacuccgaagaaauaagauccuucccug

SEQ ID NO: 31 - DNA, 119 bp, Artificial
  hairpin RNA targeted to GFP and influenza virus NP transcripts
cgggguugcgcuccuggacguagccgcagcgggguggaucuuauuucuucggaggcagguccacuccgaagaaauaagauccuucccuguccaggcuacguccaggagcgcauucccug

SEQ ID NO: 32 - DNA, 51 bp, Artificial
  shRNA targeted to infuenza virus NP transcript
ggaucuuauuucuucggagauucaagagaucuccgaagaaauaagauccuu

(19) United States
(12) Patent Application Publication
(10) Pub. No.: US 2005/0008617 A1
(43) Pub. Date: Jan. 13, 2005

(54) Compositions and methods for delivery of short interfering RNA and short hairpin RNA

(75) Inventors: Jianzhu Chen, Brookline, MA (US); Herman N. Eisen, Waban, MA (US); Qing Ge, Cambridge, MA (US)

Correspondence Address: Choate, Hall & Stewart, Exchange Place, 53 State Street, Boston, MA, 02109, US

(73) Assignee: Massachusetts Institute of Technology

(21) Appl. No.: 10/674,087

(22) Filed: Sep. 29, 2003

Related U.S. Application Data
(60) Provisional application No. 60/414,457, filed on Sep. 28, 2002.
(60) Provisional application No. 60/446,377, filed on Feb. 10, 2003.

(51) Int. Cl. A61K 48/00

(52) U.S. Cl. 424/93.2; 514/44

(57) ABSTRACT

The present invention provides compositions comprising an RNAi-inducing entity any of a variety of different delivery agents. Preferred RNAi-inducing agents include siRNA, shRNA, and RNAi-inducing vectors. Preferred delivery agents include cationic polymers, modified cationic polymers, lipids, and surfactants suitable for introduction into the lung. The invention further provides methods of inhibiting expression of a target transcript in a mammal and methods of treating or preventing a disease or condition in a mammal by administration of the compositions.

### CROSS-REFERENCE TO RELATED APPLICATION

This application claims priority to U.S. Provisional Patent Application 60/414,457, filed Sep. 28, 2002, and U.S. Provisional Patent Application 60/446,377, filed Feb. 10, 2003. The contents of each of these applications is incorporated herein by reference.

### GOVERNMENT SUPPORT

The United States Government has provided grant support utilized in the development of the present invention. In particular, National Institutes of Health grant numbers 5-RO1-AI44477, 5-RO1-AI44478, 5-ROI-CA60686, 1-RO1-AI50631, and RO1-AI40146 have supported development of this invention. The United States Government may have certain rights in the invention.

### BACKGROUND OF THE INVENTION

RNA interference (RNAi) is a process by which double-stranded RNA (dsRNA) directs sequence-specific degradation of RNA transcripts such as messenger RNA (mRNA) (Sharp, 2001; Vaucheret et al., 2001). This phenomenon was initially observed in plants (Baulcombe, 2002; Vaucheret et al., 2001) and in C. elegans (Fire et al., 1998). In plants, it appears to be an evolutionarily conserved response to virus infection. Naturally occurring RNAi is initiated by the dsRNA-specific endonuclease, called DICER, which processively cleaves long dsRNA into double-stranded fragments between 21 and 25 nucleotides long, termed short interfering RNA (siRNA) (Elbashir et al., 2001).

Studies in Drosophila showed that DICER processes long dsRNA into siRNAs comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs (WO 01/75164; Bernstein et al., Nature 409:363, 2001). siRNAs are then incorporated into a protein complex that recognizes and cleaves target mRNAs. Homologs of the DICER enzyme occur in diverse species ranging from E. coli to humans (Sharp, 2001; Zamore, Nat. Struct. Biol. 8:746, 2001), raising the possibility that an RNAi-like mechanism might be able to silence gene expression in a variety of different cell types including mammalian, or even human, cells.

Subsequently it was discovered that RNAi can be triggered in mammalian cells by introducing synthetic 21-nucleotide siRNA duplexes (Elbashir et al., 2001), bypassing the requirement for Dicer-mediated processing of long dsRNA. In mammalian cell culture, RNAi has been shown to operate in a wide variety of different cell types when synthetic oligonucleotides are introduced into cells by techniques such as transfection (Elbashir et al., 2001). Because 21 nucleotide siRNAs are too short to induce an interferon response in mammalian cells (Kumar and Carmichael, 1998), yet still able to interfere with gene expression in a sequence specific manner, they represent a new class of molecules that may have significant applications in fields ranging from functional genomics to medicine. For example, it has been shown that RNAi induces degradation of respiratory syncytial virus in culture (Bitko and Barik, 2001). However, in order to fully realize the potential of siRNA, it would be desirable to be able to induce the phenomenon in larger eukaryotic organisms, e.g., mammals and birds. Thus there is a need in the art for compositions and methods for delivery of siRNA and related molecules to mammalian and avian cells within intact organisms. The present invention addresses this need among others.

### SUMMARY OF THE INVENTION

The present invention provides novel compositions to facilitate the delivery of RNAi-inducing entities such as short interfering siRNAs (siRNAs), short hairpin RNAs (shRNAs), and RNAi-inducing vectors (i.e., vectors whose presence within a cell results in production of an siRNA or shRNA) to cells, tissues, and organs in living birds and mammals, e.g., humans. In particular, the invention provides compositions comprising an RNAi-inducing entity, wherein the RNAi-inducing entity is targeted to one or more target transcripts and any of a variety of delivery agents. In various embodiments of the invention the RNAi-inducing entity can be an siRNA, shRNA, or RNAi-inducing vector. In certain embodiments of the invention the composition comprises an siRNA comprising two RNA strands having a region of complementarity approximately 19 nucleotides in length and optionally further comprises one or two single-stranded overhangs or loops. In certain embodiments of the invention the composition comprises an shRNA comprising a single RNA strand having a region of self-complementarity. The single RNA strand may form a hairpin structure with a stem and loop and, optionally, one or more unpaired portions at the 5′ and/or 3′ portion of the RNA.

Thus in one aspect, the invention provides a composition comprising: (i) an RNAi-inducing entity, wherein the RNAi-inducing entity is targeted to a target transcript; and (ii) a delivery agent selected from the group consisting of: cationic polymers, modified cationic polymers, peptide molecular transporters, surfactants suitable for introduction into the lung, liposomes, non-cationic polymers, modified non-cationic polymers, bupivacaine, and chloroquine. In certain embodiments of the invention the delivery agent incorporates a delivery-enhancing moiety to enhance delivery or specificity of delivery to a cell of interest. In various embodiments of the invention the RNAi-inducing entity can be an siRNA, sHRNA, or RNAi-inducing vector.

In another aspect, the invention provides a method of inhibiting expression of a target transcript in a mammalian subject comprising the step of administering to the subject a composition comprising: (i) an RNAi-inducing entity, wherein the RNAi-inducing entity is targeted to the target transcript; and (ii) a delivery agent selected from the group consisting of: cationic polymers, modified cationic polymers, peptide molecular transporters, surfactants suitable for introduction into the lung, lipids, liposomes, non-cationic polymers, modified non-cationic polymers, bupivacaine, and chloroquine. In various embodiments of the invention the compositions are administered intravenously or by introduction into the lung.

The methods may be applied for a variety of purposes, e.g, to study the function of the transcript, to study the effect of different compounds on a cell or organism in the absence (or reduced activity) of the polypeptide encoded by the transcript, etc. Knowledge of the effect of inhibiting expression of a transcript allows determination of whether the gene from which the transcript is transcribed is a suitable pharmaceutical target for treatment of diseases involving the gene and/or the biological pathway(s) in which it plays a role, or for therapeutic purposes.

The present invention further provides methods of treating or preventing diseases or conditions associated with excessive expression (overexpression) or inappropriate expression of a target transcript or inappropriate or excessive functional activity of a polypeptide encoded by the target transcript, or of providing symptomatic relief, by administering inventive compositions to a subject at risk of or suffering from such a condition within an appropriate time window prior to, during, or after the onset of symptoms.

This application refers to various patents, journal articles, and other publications, all of which are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Goldsby, R. A, et al., Kuby Immunology, 4^{th} ed., W.H. Freeman and Co., New York, 2000; Goodman and Gilman 's The Pharmacological Basis of Therapeutics, 10^{th} ed., McGraw-Hill, 2001 and Physician's Desk Reference, 56^{th} ed., ISBN: 1563634112 Medical Economics, 2002.

### BRIEF DESCRIPTION OF THE DRAWING

FIG. 1 shows the structure of siRNAs observed in the Drosophila system.

FIG. 2 presents a schematic representation of the steps involved in RNA interference in Drosophila.

FIG. 3 shows a variety of exemplary siRNA or shRNA structures useful in accordance with the present invention.

FIG. 4 presents a representation of an alternative inhibitory pathway, in which the DICER enzyme cleaves a substrate having a base mismatch in the stem to generate an inhibitory product that binds to the 3′ UTR of a target transcript and inhibits translation.

FIG. 5 presents one example of a construct that may be used to direct transcription of both strands of an inventive siRNA.

FIG. 6 depicts one example of a construct that may be used to direct transcription of an shRNA according to the present invention.

FIG. 7A shows schematic diagrams of NP-1496 and GFP-949 siRNA and their hairpin derivatives/precursors.

FIG. 7B shows tandem arrays of NP-1496 and GFP-949 in two different orders.

FIG. 7C shows pSLOOP III expression vectors. Hairpin precursors of siRNA are cloned in the pSLOOP III vector alone (top), in tandem arrays (middle), or simultaneously with independent promoter and termination sequence (bottom).

FIG. 8A is a plot showing that siRNA inhibits influenza virus production in mice when administered prior to infection with influenza virus. Filled squares (no treatment); Open squares (GFP siRNA); Open circles (30 μg NP siRNA); Filled circles (60 μg NP siRNA). Each symbol represents an individual animal. p values between different groups are shown.

FIG. 8B is a plot showing that siRNA inhibits influenza virus production in mice when administered together with the cationic polymer PLL prior to infection with influenza virus. Filled squares (no treatment); Open squares (GFP siRNA); Filled circles (60 μg NP siRNA). Each symbol represents an individual animal. p values between different groups are shown.

FIG. 8C is a plot showing that siRNA inhibits influenza virus production in mice when administered together with the cationic polymer jetPEI prior to infection with influenza virus significantly more effectively than when administered in PBS. Open squares (no treatment); Open triangles (GFP siRNA in PBS); Filled triangles (NP siRNA in PBS); Open circles (GFP siRNA with jetPEI); Filled circles (NP siRNA with jetPEI). Each symbol represents an individual animal. p values between different groups are shown.

FIG. 9 is a plot showing that siRNAs targeted to influenza virus NP and PA transcripts exhibit an additive and/or synergistic effect when administered together prior to infection with influenza virus. Filled squares (no treatment); Open circles (60 μg NP siRNA); Open triangles (60 μg PA siRNA); Filled circles (60 μg NP siRNA+60 μg PA siRNA). Each symbol represents an individual animal. p values between different groups are shown.

FIG. 10 is a plot showing that siRNA inhibits influenza virus production in mice when administered following infection with influenza virus. Filled squares (no treatment); Open squares (60 μg GFP siRNA); Open triangles (60 μg PA siRNA); Open circles (60 μg NP siRNA); Filled circles (60 μg NP+60 μg PA siRNA). Each symbol represents an individual animal. p values between different groups are shown.

FIG. 11A is a schematic diagram of a lentiviral vector expressing a shRNA. Transcription of shRNA is driven by the U6 promoter. EGFP expression is driven by the CMV promoter. SIN-LTR, Ψ, cPPT, and WRE are lentivirus components. The sequence of NP-1496 shRNA is shown.

FIG. 11B presents plots of flow cytometry results demonstrating that Vero cells infected with the lentivirus depicted in FIG. 25B express EGFP in a dose-dependent manner. Lentivirus was produced by co-transfecting DNA vector encoding NP-1496 shRNA and packaging vectors into 293T cells. Culture supernatants (0.25 ml or 1.0 ml) were used to infect Vero cells. The resulting Vero cell lines (Vero-NP-0.25 and Vero-NP-1.0) were analyzed for GFP expression by flow cytometry. Mean fluorescence intensity of Vero-NP-0.25 (left) and Vero-NP-1.0 (right) cells are shown.

FIG. 11C is a plot showing inhibition of influenza virus production in Vero cells that express NP-1496 shRNA. Parental and NP-1496 shRNA expressing Vero cells were infected with PR8 virus at MOI of 0.04, 0.2 and 1. Virus titers in the supernatants were determined by hemagglutination (HA) assay 48 hrs after infection.

FIG. 12 is a plot showing that influenza virus production in mice is inhibited by administration of DNA vectors that express siRNA targeted to influenza virus transcripts. Sixty μg of DNA encoding RSV, NP-1496 (NP) or PB1-2257 (PB1) shRNA were mixed with 40 μl Infasurf and were administered into mice by instillation. For no treatment (NT) group, mice were instilled with 60 μl of 5% glucose. Thirteen hrs later, the mice were infected intranasally with PR8 virus, 2000 pfu per mouse. The virus titers in the lungs were measured 24 hrs after infection by MDCK/hemagglutinin assay. Each data point represents one mouse. p values between groups are indicated.

FIG. 13A shows results of an electrophoretic mobility shift assay for detecting complex formation between siRNA and poly-L-lysine (PLL). SiRNA-polymer complexes were formed by mixing 150 ng of NP-1496 siRNA with increasing amounts of polymer (0-1200 ng) for 30 min at room temperature. The reactive mixtures were then run on a 4% agarose gel and siRNAs were visualized with ethidium-bromide staining.

FIG. 13B shows results of an electrophoretic mobility shift assay for detecting complex formation between siRNA and poly-L-arginine (PLA). SiRNA-polymer complexes were formed by mixing 150 ng of NP-1496 siRNA with increasing amounts of polymer (0-1200 ng) for 30 min at room temperature. The reactive mixtures were then run on a 4% agarose gel and siRNAs were visualized with ethidium-bromide staining.

FIG. 14A is a plot showing cytotoxicity of siRNA/PLL complexes. Vero cells in 96-well plates were treated with siRNA (400 pmol)/polymer complexes for 6 hrs. The polymer-containing medium was then replaced with DMEM-10% FCS. The metabolic activity of the cells was measured 24 h later by using the MTT assay. Squares=PLL (MW ˜8K); Circles=PLL (MW ˜42K) Filled squares=25%; Open triangles=50%; Filled triangles=75%; X=95%. The data are shown as the average of triplicates.

FIG. 14B is a plot showing cytotoxicity of siRNA/PLA complexes. Vero cells in 96-well plates were treaed with siRNA (400 pmol)/polymer complexes for 6 hrs. The polymer-containing medium was then replaced with DMEM-10% FCS. The metabolic activity of the cells was measured 24 h later by using the MTT assay. The data are shown as the average of triplicates.

FIG. 15A is a plot showing that PLL stimulates cellular uptake of siRNA. Vero cells in 24-well plates were incubated with Lipofectamine+siRNA (400 pmol) or with siRNA (400 pmol)/polymer complexes for 6 hrs. The cells were then washed and infected with PR8 virus at a MOI of 0.04. Virus titers in the culture supernatants at different time points after infection were measured by HA assay. Polymer to siRNA ratios are indicated. Open circles=no treatment; Filled squares=Lipofectamine; Filled triangles=PLL (MW ˜42K); Open triangles=PLL (MW ˜8K).

FIG. 15B is a plot showing that poly-L-arginine stimulates cellular uptake of siRNA. Vero cells in 24-well plates were incubated with siRNA (400 pmol)/polymer complexes for 6 hrs. The cells were then washed and infected with PR8 virus at a MOI of 0.04. Virus titers in the culture supernatants at different time points after infection were measured by HA assay. Polymer to siRNA ratios are indicated. 0, 25, 50, 75, and 95% refer to percentage of ε-amino groups on PLL substituted with imidazole acetyl groups. Closed circles=no transfection; Open circles=Lipofectamine; Open and filled squares=0% and 25% (Note that the data points for 0% and 25% are identical); Filled triangles=50%; Open triangles=75%; X=95%.

FIG. 16A is a schematic of a developing chicken embryo indicating the area for injection of siRNA and siRNA/delivery agent compositions.

FIG. 16B shows the ability of various siRNAs to inhibit influenza virus production in developing chicken embryos when delivered in combination with a lipid-based delivery agent.

### DEFINITIONS

In general, the term antibody refers to an immunoglobulin, whether natural or wholly or partially synthetically produced. In certain embodiments of the invention the term also encompasses any protein comprising a immunoglobulin binding domain. These proteins may be derived from natural sources, or partly or wholly synthetically produced. The antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol.2, 750-765, 2002, and references therein. In certain embodiments of the invention the term includes “humanized” antibodies in which for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. An antibody may be polyclorial or monoclonal, though for purposes of the present invention monoclonal antibodies are generally preferred.

As used herein, the terms approximately or about in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

By associated with, characterized by, or featuring excessive or inappropriate expression of a transcript or polypeptide is generally meant that excessive or inappropriate expression of the transcript or polypeptide frequently (e.g., in a majority of instances), typically, or consistently occurs in the presence of the disease or condition. It is not necessary that excessive or inappropriate expression invariably occurs in the presence of the disease or condition, and in fact excessive or inappropriate expression may only occur in a small subset (e.g., less than 5%) of the subjects suffering from the disease or condition). In general, the excessive or inappropriate expression of the transcript or polypeptide either directly or indirectly causes or contributes to the disease or condition or a symptom thereof. It is noted that whether or not expression or activity is excessive or inappropriate may depend on context. For example, expression of a receptor for a ligand may have no effect in the absence of the ligand while in the presence of the ligand such expression may be deemed excessive or inappropriate if it results in a disease or symptom. In the therapeutic context, the phrases associated with, characterized by, or featuring generally mean that at least one symptom of the condition or disease to be treated is caused, exacerbated, or contributed to by the transcript or encoded polypeptide, such that a reduction in the expression of the transcript or polypeptide will alleviate, reduce, or prevent one or more features or symptoms of the disease or condition.

The term gene has its meaning as understood in the art. However, it will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs. For the purpose of clarity we note that, as used in the present application, the term gene generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

A gene product or expression product is, in general, an RNA transcribed from the gene (either pre-or post-processing) or a polypeptide (either pre- or post-modification) encoded by an RNA transcribed from the gene.

The term hybridize, as used herein, refers to the interaction between two complementary nucleic acid sequences. The phrase hybridizes under high stringency conditions describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6× sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.; high stringency hybridization (e.g., 6×SSC at about 45° C., followed by one or more washes in 0.2×SSC, 0.1% SDS at 65° C.; and very high stringency hybridization conditions (e.g., 0.5M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based upon various factors such as the length of the hybridizing sequences, whether they contain RNA or DNA, etc. For example, appropriate temperatures for high, medium, or low stringency hybridization will generally be lower for shorter sequences such as oligonucleotides than for longer sequences.

Inappropriate or excessive, as used herein in reference to the expression of a transcript or the functional activity of a polypeptide or cell refers to expression or activity that either (i) occurs at a level higher than occurs normally in a wild type cell or healthy subject or under typical environmental conditions, typically a level that contributes to or causes a detectable result such as a symptom or sign of disease; or (ii) occurs in a temporal or spatial pattern that differs from that which occurs normally in a wild type cell or healthy subject or under typical environmental conditions. The term includes expression or activity in a cell type that does not normally exhibit expression or activity. Whether or not a cell or subject exhibits inappropriate or excessive expression of a transcript or inappropriate or excessive activity of a polypeptide or inappropriate or excessive cellular functional activity may be determined, for example, by comparing the expression or activity either with wild type or normal subjects or with historical controls.

Isolated, as used herein, means 1) separated from at least some of the components with which it is usually associated in nature; 2) prepared or purified by a process that involves the hand of man; and/or 3) not occurring in nature.

Ligand, as used herein, means a molecule that specifically binds to a second molecule, typically a polypeptide or portion thereof, such as a carbohydrate moiety, through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity (e.g., agonists or antagonists), the term as used herein applies more generally.

Operably linked, as used herein, refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Purified, as used herein, means separated from many other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

The term regulatory sequence is used herein to describe a region of nucleic acid sequence that directs, enhances, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J. 8:729, 1989) and immunoglobulins (see, for example, Baneiji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention regulatory sequences may direct expression of a nucleotide sequence only in cells that have been infected with an infectious agent. For example, the regulatory sequence may comprise a promoter and/or enhancer such as a virus-specific promoter or enhancer that is recognized by a viral protein, e.g., a viral polymerase, transcription factor, etc. Alternately, the regulatory sequence may comprise a promoter and/or enhancer that is active in epithelial cells in the nasal passages, respiratory tract and/or the lungs.

As used herein, the term RNAi-inducing entity encompasses RNA molecules and vectors whose presence within a cell results in RNAi and leads to reduced expression of a transcript to which the RNAi-inducing entity is targeted. The term specifically includes siRNA, shRNA, and RNAi-inducing vectors.

As used herein, an RNAi-inducing vector is a vector whose presence within a cell results in transcription of one or more RNAs that self-hybridize or hybridize to each other to form an shRNA or siRNA. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in production of one or more RNAs that self-hybridize or hybridize to each other to form an shRNA or siRNA. In general, the vector comprises a nucleic acid operably linked to expression signal(s) so that one or more RNA molecules that hybridize or self-hybridize to form an siRNA or shRNA are transcribed when the vector is present within a cell. Thus the vector provides a template for intracellular synthesis of the RNA or RNAs or precursors thereof. For purposes of inducing RNAi, presence of a viral genome into a cell (e.g., following fusion of the viral envelope with the cell membrane) is considered sufficient to constitute presence of the virus within the cell. In addition, for purposes of inducing RNAi, a vector is considered to be present within a cell if it is introduced into the cell, enters the cell, or is inherited from a parental cell, regardless of whether it is subsequently modified or processed within the cell. An RNAi-inducing vector is considered to be targeted to a transcript if presence of the vector within a cell results in production of one or more RNAs that hybridize to each other or self-hybridize to form an siRNA or shRNA that is targeted to the transcript, i.e., if presence of the vector within a cell results in production of one or more siRNAs or shRNAs targeted to the transcript.

A short, interfering RNA (siRNA) comprises an RNA duplex that is approximately 19 basepairs long and optionally further comprises one or two single-stranded overhangs. An siRNA may be formed from two RNA molecules that hybridize together, or may alternatively be generated from a single RNA molecule that includes a self-hybridizing portion. It is generally preferred that free 5′ ends of siRNA molecules have phosphate groups, and free 3′ ends have hydroxyl groups. The duplex portion of an siRNA may, but typically does not, contain one or more bulges consisting of one or more unpaired nucleotides. One strand of an siRNA includes a portion that hybridizes with a target transcript. In certain preferred embodiments of the invention, one strand of the siRNA is precisely complementary with a region of the target transcript, meaning that the siRNA hybridizes to the target transcript without a single mismatch. In other embodiments of the invention one or more mismatches between the siRNA and the targeted portion of the target transcript may exist. In most embodiments of the invention in which perfect complementarity is not achieved, it is generally preferred that any mismatches be located at or near the siRNA termini.

The term short hairpin RNA refers to an RNA molecule comprising at least two complementary portions hybridized or capable of hybridizing to form a double-stranded (duplex) structure sufficiently long to mediate RNAi (typically at least 19 base pairs in length), and at least one single-stranded portion, typically between approximately 1 and 10 nucleotides in length that forms a loop. The duplex portion may, but typically does not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript.

As used herein, the term specific binding refers to an interaction between a target polypeptide (or, more generally, a target molecule) and a binding molecule such as an antibody, ligand, agonist, or antagonist. The interaction is typically dependent upon the presence of a particular structural feature of the target polypeptide such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute but generally refers to the context in which the binding is performed. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target polypeptide versus the affinity of the binding molecule for other targets, e.g., competitors. If a binding molecule exhibits a high affinity for a target molecule that it is desired to detect and low affinity for nontarget molecules, the antibody will likely be an acceptable reagent for immunodiagnostic purposes. Once the specificity of a binding molecule is established in one or more contexts, it may be employed in other, preferably similar, contexts without necessarily re-evaluating its specificity.

The term subject, as used herein, refers to an individual susceptible to infection with an infectious agent, e.g., an individual susceptible to infection with an virus such as the influenza virus. The term includes animals, e.g., domesticated animals (such as chickens, swine, horse, dogs, cats, etc.), and wild animals, non-human primates, and humans.

An siRNA or shRNA or an siRNA or shRNA sequence is considered to be targeted to a target transcript for the purposes described herein if I) the stability of the target transcript is reduced in the presence of the siRNA or shRNA as compared with its absence; and/or 2) the siRNA or shRNA shows at least about 90%, more preferably at least about 91%, 92%, 93%, 94%, 95%, 96%, 97%, 98%, 99%, or 100% precise sequence complementarity with the target transcript for a stretch of at least about 15, more preferably at least about 17, yet more preferably at least about 18 or 19 to about 21-23 nucleotides; and/or 3) one strand of the siRNA or one of the self-complementary portions of the shRNA hybridizes to the target transcript under stringent conditions for hybridization of small (<50 nucleotide) RNA molecules in vitro and/or under conditions typically found within the cytoplasm or nucleus of mammalian cells. An RNA-inducing vector whose presence within a cell results in production of an siRNA or shRNA that is targeted to a transcript is also considered to be targeted to the target transcript. Since the effect of targeting a transcript is to reduce or inhibit expression of the gene that directs synthesis of the transcript, an siRNA, shRNA, or RNAi-inducing vector targeted to a transcript is also considered to target the gene that directs synthesis of the transcript even though the gene itself (i.e., genomic DNA) is not thought to interact with the siRNA, shRNA, or components of the cellular silencing machinery. Thus as used herein, an siRNA, shRNA, or RNAi-inducing vector that targets a transcript is understood to target the gene that provides a template for synthesis of the transcript.

As used herein, treating includes reversing, alleviating, inhibiting the progress of, preventing, or reducing the likelihood of the disease, disorder, or condition to which such term applies, or one or more symptoms or manifestations of such disease, disorder or condition.

In general, the term vector refers to a nucleic acid molecule capable of mediating entry of, e.g., transferring, transporting, etc., a second nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (typically DNA molecules although RNA plasmids are also known), cosmids, and viral vectors. As is well known in the art, the term viral vector may refer either to a nucleic acid molecule (e.g., a plasmid) that includes virus-derived nucleic acid elements that typically facilitate transfer or integration of the nucleic acid molecule (examples include retroviral or lentiviral vectors) or to a virus or viral particle that mediates nucleic acid transfer (examples include retroviruses or lentiviruses). As will be evident to one of ordinary skill in the art, viral vectors may include various viral components in addition to nucleic acid(s).

### DETAILED DESCRIPTION OF CERTAIN PREFERRED EMBODIMENTS OF THE INVENTION

I. Overview

The present invention provides novel compositions to facilitate the delivery of RNAi-inducing entities such as short interfering siRNAs (siRNAs), short hairpin RNAs (shRNAs), and/or RNAi-inducing vectors to cells, tissues, and organs in living mammals, e.g., humans. In particular, the invention provides compositions comprising (i) one or more RNAi-inducing entities, wherein the one or more RNAi-inducing entities are targeted to one or more target transcripts; and (ii) a delivery agent selected from the group consisting of: cationic polymers, modified cationic polymers, peptide molecular transporters, surfactants suitable for introduction into the lung, neutral or cationic lipids, liposomes, non-cationic polymers, modified non-cationic polymers, chloroquine, and bupivacaine. While it is noted that the inventive compositions may find particular utility for delivery of RNAi-inducing entities such as siRNA, shRNA, or RNAi-inducing vectors to cells in intact mammalian subjects, they may also be used for delivery of these agents to cells in tissue culture. Indeed in general it may be desirable to test the safety and/or efficacy of inventive compositions comprising one or more RNAi-inducing entities using cells in tissue culture prior to introducing the compositions into living subjects.

The invention also provides methods for the delivery of one or more RNAi-inducing entities to organs and tissues within the body of a mammal, e.g., a human. In one embodiment of the invention compositions comprising an RNAi-inducing entity and a cationic polymer are introduced into a blood vessel (i.e., intravascularly), preferably into a vein, although arterial delivery is also within the scope of the invention. The one or more RNAi-inducing entity is transported within the body and taken up by cells in one or more organs or tissues, where it inhibits expression of a target transcript. In one embodiment of the invention the organ is the lung.

In another embodiment of the invention compositions comprising an one or more RNAi-inducing entities and a surfactant are introduced into the lung. The siRNA or shRNA is taken up by cells in the lung, where they inhibit expression of a target transcript. In a related embodiment of the invention a compositions comprising an RNAi-inducing entity is introduced into the lung and transported from the lung to other sites within the body, where it inhibits expression of a target transcript. The following sections describe the features of siRNAs and shRNAs for use in the invention and provide further details of the delivery agents and methods of use of the compositions.

II. siRNA and shRNA Features

In general, siRNAs and shRNAs may be designed to inhibit virtually any target transcript in mammalian cells. Whatever transcript target is selected, the design of siRNAs and shRNAs for use in accordance with the present invention will preferably follow certain guidelines. In general, it is preferable to target sequences that are specific to the transcript whose inhibition is desired. Also, in many cases, the RNAi-inducing entity that is delivered to a cell or subject according to the present invention may undergo one or more processing steps before becoming an active suppressing agent (see below for further discussion); in such cases, those of ordinary skill in the art will appreciate that the relevant agent will preferably be designed to include sequences that may be necessary for its processing.

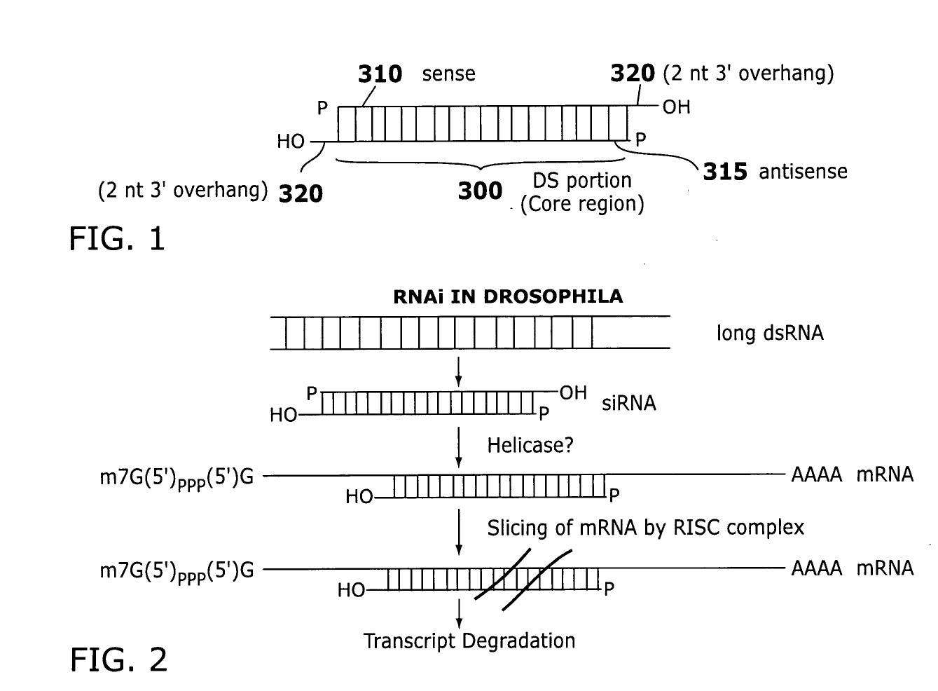

As mentioned above, small inhibitory RNAs were first discovered in studies of the phenomenon of RNA interference (RNAi) in Drosophila, as described in WO 01/75164. In particular, it was found that, in Drosophila, long double-stranded RNAs are processed by an RNase III-like enzyme called DICER (Bernstein et al., Nature 409:363, 2001) into smaller dsRNAs comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. FIG. 1 shows a schematic of siRNAs found in Drosophila. The structure includes a 19 nucleotide double-stranded (DS) portion 300, comprising a sense strand 310 and an antisense strand 315. Each strand has a 2 nt 3′ overhang 320.

These small dsRNAs (siRNAs) act to silence expression of any gene that includes a region complementary to one of the dsRNA strands, presumably because a helicase activity unwinds the 19 bp duplex in the siRNA, allowing an alternative duplex to form between one strand of the siRNA and the target transcript. This new duplex then guides an endonuclease complex, RISC, to the target RNA, which it cleaves (“slices”) at a single location, producing unprotected RNA ends that are promptly degraded by cellular machinery (FIG. 2). As mentioned below, additional mechanisms of silencing mediated by short RNA species (micrORNAs) are also known (see, e.g., Ruvkun, G., Science, 294, 797-799, 2001; Zeng, Y., et al., Molecular Cell, 9, 1-20, 2002). It is noted that the discussion of mechanisms and the figures depicting them are not intended to suggest any limitations on the mechanism of action of the present invention.

The inventors and others have found that siRNAs and shRNAs, and vectors whose presence within a cell results in production of siRNAs or shRNAs, can effectively reduce the expression of target genes when introduced into mammalian cells. As described in copending U.S. patent application entitled “Influenza Therapeutic”, filed on even date herewith, and incorporated by reference herein, the inventors have shown that siRNAs targeted to a variety of cellular transcripts greatly reduced the level of the target transcript in mammalian cells. In addition, as described in the Examples, the inventors have shown that siRNAs targeted to a variety of viral RNAs inhibited the production of influenza virus in tissue culture cells and in mice. Furthermore, the inventors have shown that administration of a DNA vector from which siRNA precursors (shRNAs) can be expressed inhibits influenza virus production in mice. These results demonstrate that treatment with siRNA, shRNA, or with vectors whose presence within a cell leads to expression of siRNA or shRNA is an effective strategy for reducing or inhibiting the expression of target transcripts and for treating or preventing diseases associated with, characterized by, or featuring excessive or inappropriate expression of particular transcripts or inappropriate or excessive expression or functional activity of a polypeptide encoded by the transcript.

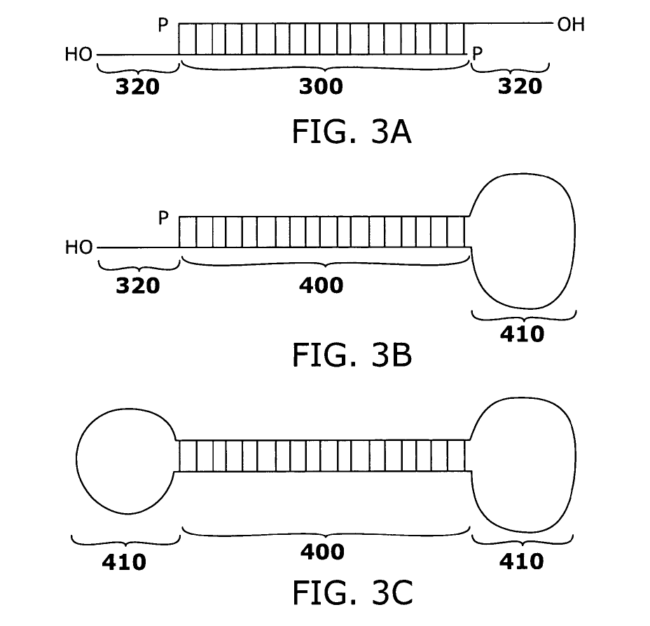

Preferred siRNAs and shRNAs for use in accordance with the present invention include a base-paired region approximately 19 nt long, and may optionally have free or looped ends. For example, FIG. 3 presents various structures that could be utilized as an siRNA or shRNA according to the present invention. FIG. 3A shows the structure found to be active in the Drosophila system described above, which is likely to represent a species that is active in mammalian cells. The present invention encompasses administration of an siRNA having the structure depicted in FIG. 3A to mammalian cells in order to treat or prevent a disease or condition associated with inappropriate or excessive expression of a target transcript or inappropriate or excessive expression or functional activity of a polypeptide encoded by the transcript. However, it is not required that the administered agent have this structure. For example, the administered composition may include any structure capable of being processed in vivo to the structure of FIG. 3A, so long as the administered agent does not induce detrimental effects such as induction of the interferon response. (Note that the term in vivo, as used herein with respect to the synthesis, processing, or activity of siRNA or shRNA, generally refers to events that occur within a cell as opposed to in a cell-free system. In general, the cell can be maintained in tissue culture or can be part of an intact organism.) The invention may also comprise administration of agents that are not processed to precisely the structure depicted in FIG. 3A, so long as administration of such agents reduces target transcript levels sufficiently as discussed herein.

FIGS. 3B and 3C present two alternative structures that may be used to mediate RNAi in accordance with the present invention. These hairpin (stem-loop) structures may function directly as inhibitory RNAs or may be processed intracellularly to yield an siRNA structure such as that depicted in FIG. 3A. FIG. 3B shows an agent comprising an RNA strand containing two complementary elements that hybridize to one another to form a duplex region represented as stem 400, a loop 410, and an overhang 320. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. One of ordinary skill in the art will appreciate that loops of 4 nucleotides or greater are less likely subject to steric constraints than are shorter loops and therefore may be preferred. In some embodiments, the overhang includes a 5′ phosphate and a 3′ hydroxyl. As discussed below, an agent having the structure depicted in FIG. 3B can readily be generated by transcription within cells or by in vitro transcription; in several preferred embodiments, the transcript tail will be included in the overhang, so that often the overhang will comprise a plurality of U residues, e.g., between 1 and 5 U residues. It is noted that synthetic siRNAs that have been studied in mammalian systems often have 2 overhanging U residues. The loop may be located at either the 5′ or 3′ end of the portion that is complementary to the target transcript whose inhibition is desired (i.e., the antisense portion of the shRNA).

FIG. 3C shows an agent comprising an RNA circle that includes complementary elements sufficient to form a stem 400 approximately 19 bp long. Such an agent may show improved stability as compared with various other siRNAs described herein.

In describing siRNAs it will frequently be convenient to refer to sense and antisense strands of the siRNA. In general, the sequence of the duplex portion of the sense strand of the siRNA is substantially identical to the targeted portion of the target transcript, while the antisense strand of the siRNA is substantially complementary to the target transcript in this region as discussed further below. Although shRNAs contain a single RNA molecule that self-hybridizes, it will be appreciated that the resulting duplex structure may be considered to comprise sense and antisense strands or portions. It will therefore be convenient herein to refer to sense and antisense strands, or sense and antisense portions, of an shRNA, where the antisense strand or portion is that segment of the molecule that forms or is capable of forming a duplex and is substantially complementary to the targeted portion of the target transcript, and the sense strand or portion is that segment of the molecule that forms or is capable of forming a duplex and is substantially identical in sequence to the targeted portion of the target transcript.

For purposes of description, the discussion below may refer to siRNA rather than to siRNA or shRNA. However, as will be evident to one of ordinary skill in the art, teachings relevant to the sense and antisense strand of an siRNA are generally applicable to the sense and antisense portions of the stem portion of a corresponding shRNA. Thus in general the considerations below apply also to shRNAs.

It will be appreciated by those of ordinary skill in the art that agents having any of the structures depicted in FIG. 3, or any other effective structure as described herein, may be comprised entirely of natural RNA nucleotides, or may instead include one or more nucleotide analogs. A wide variety of such analogs is known in the art; the most commonly-employed in studies of therapeutic nucleic acids being the phosphorothioate (for some discussion of considerations involved when utilizing phosphorothioates, see, for example, Agarwal, Biochim. Biophys. Acta 1489:53, 1999). In particular, in certain embodiments of the invention it may be desirable to stabilize the siRNA structure, for example by including nucleotide analogs at one or more free strand ends in order to reduce digestion, e.g., by exonucleases. The inclusion of deoxynucleotides, e.g., pyrimidines such as deoxythymidines at one or more free ends may serve this purpose. Alternatively or additionally, it may be desirable to include one or more nucleotide analogs in order to increase or reduce stability of the 19 bp stem, in particular as compared with any hybrid that will be formed by interaction of one strand of the siRNA with a target transcript.

According to certain embodiments of the invention various nucleotide modifications are used selectively in either the sense or antisense strand. For example, it may be preferable to utilize unmodified ribonucleotides in the antisense strand while employing modified ribonucleotides and/or modified or unmodified deoxyribonucleotides at some or all positions in the sense strand. According to certain embodiments of the invention only unmodified ribonucleotides are used in the duplex portion of the antisense and/or the sense strand of the siRNA while the overhang(s) of the antisense and/or sense strand may include modified ribonucleotides and/or deoxyribonucleotides. In particular, according to certain embodiments of the invention the sense strand contains a modification that reduces or eliminates silencing of transcripts complementary to the sense strand while not preventing silencing of transcripts complementary to the antisense strand, as described in copending U.S. Patent Application entitled “Influenza Therapeutic”, filed on even date herewith, and U.S. Provisional Patent Application 60/446,387, entitled “Methods and Reagents for Reducing Undesired Targeting of Short Interfering RNA”.

Numerous nucleotide analogs and nucleotide modifications are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared. A number of modifications have been shown to alter one or more aspects of the oligonucleotide such as its ability to hybridize to a complementary nucleic acid, its stability, etc. For example, useful 2′-modifications include halo, alkoxy and allyloxy groups. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.) “Antisense Drug Technology: Principles, Strategies, and Applications” (1^{st} ed), Marcel Dekker; ISBN: 0824705661; 1st edition (2001) and references therein. As will be appreciated by one of ordinary skill in the art, analogs and modifications may be tested using, e.g., the assays described herein or other appropriate assays, in order to select those that effectively reduce expression of viral genes. See references 116-118 for further discussion of modifications that have been found to be useful in the context of siRNA.

In certain embodiments of the invention the analog or modification results in an siRNA with increased absorbability (e.g., increased absorbability across a mucus layer, increased absorption, etc.), increased stability in the blood stream or within cells, increased ability to cross cell membranes, etc. As will be appreciated by one of ordinary skill in the art, analogs or modifications may result in altered Tm, which may result in increased tolerance of mismatches between the siRNA sequence and the target while still resulting in effective suppression.

It will further be appreciated by those of ordinary skill in the art that effective siRNA agents for use in accordance with certain embodiments of the present invention may comprise one or more moieties that is/are not nucleotides or nucleotide analogs.

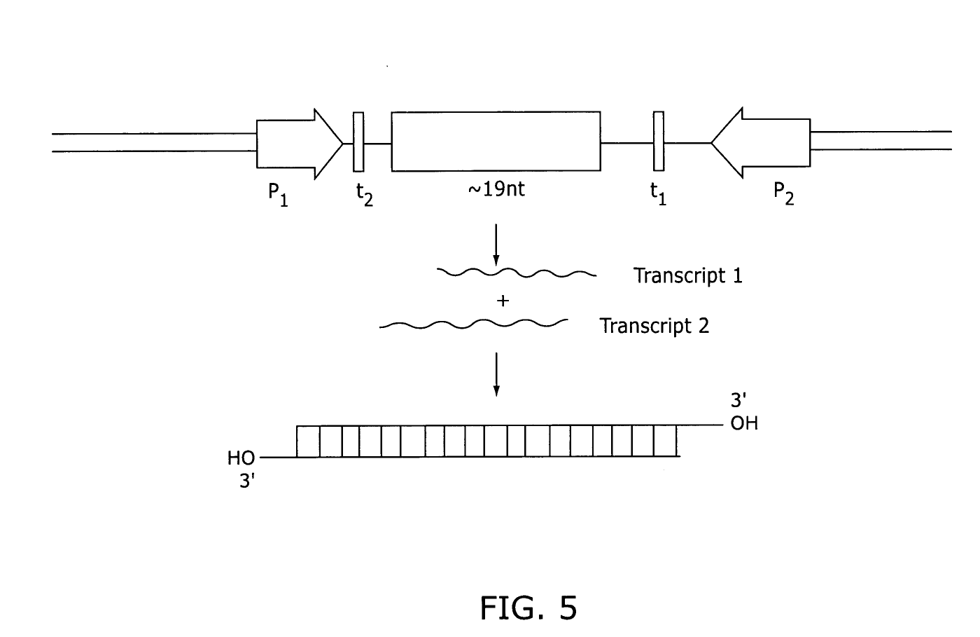

In general, one strand of inventive siRNAs will preferably include a region (the “inhibitory region”) that is substantially complementary to that found in a portion of the target transcript, so that a precise hybrid can form in vivo between one strand or portion of the siRNA (the antisense strand) and the target transcript. In those embodiments of the invention in which an shRNA structure is employed, this substantially complementary region preferably includes most or all of the stem structure depicted in FIG. 5B. In certain preferred embodiments of the invention, the relevant inhibitor region of the siRNA or shRNA is perfectly complementary with the target transcript; in other embodiments, one or more non-complementary residues are located within the siRNA/template duplex. It may be preferable to avoid mismatches in the central portion of the siRNA/template duplex (see, for example, Elbashir et al., EMBO J. 20:6877, 2001, incorporated herein by reference).

In preferred embodiments of the invention, the siRNA hybridizes with a target site that includes exonic sequences in the target transcript. Hybridization with intronic sequences is not excluded, but generally appears not to be preferred in mammalian cells. In certain preferred embodiments of the invention, the siRNA hybridizes exclusively with exonic sequences. In some embodiments of the invention, the siRNA hybridizes with a target site that includes only sequences within a single exon; in other embodiments the target site is created by splicing or other modification of a primary transcript. Any site that is available for hybridization with an siRNA resulting in slicing and degradation of the transcript may be utilized in accordance with the present invention. Nonetheless, those of ordinary skill in the art will appreciate that, in some instances, it may be desirable to select particular regions of target transcript as siRNA hybridization targets. For example, it may be desirable to avoid sections of target transcript that may be shared with other transcripts whose degradation is not desired. In general, coding regions and regions closer to the 3′ end of the transcript than to the 5′ end are preferred.

siRNAs may be selected according to a variety of approaches, and generally any art-accepted method of selection may be used. The description herein is not intended to limit the invention. In general, as mentioned above, inventive siRNAs will preferably include a region (the “inhibitory region” or “duplex region”) that is perfectly complementary or substantially complementary to that found in a portion of the target transcript (the “target portion”), so that a hybrid can form in vivo between the antisense strand of the siRNA and the target transcript. This duplex region, also referred to as the “core region” is understood not to include 3′ overhangs, although overhangs, if present, may also be complementary to the target transcript or its complement (e.g., the 3′ overhang of the antisense siRNA strand may be complementary to the target transcript and the 3′ overhang of the sense siRNA strand may be identical to the corresponding nucleotides in the target transcript, i.e., those nucleotides immediately 3′ of the target site). Preferably, this perfectly or substantially complementary region includes most or all of the duplex or stem structure depicted in FIGS. 3, 4, and 5. The relevant inhibitor region of the siRNA is preferably perfectly complementary with the target transcript. However, siRNAs including one or more non-complementary residues have also been shown to mediate silencing, though the extent of inhibition may be less than that achievable using siRNAs with duplex portions that are perfectly complementary to the target transcript.

For purposes of description herein, the length of an siRNA core region will be assumed to be 19 nucleotides, and a 19 nucleotide sequence is referred to as N19. However, the core region may range in length from 15 to 29 nucleotides. Typically the length of each of the two strands is approximately between 21 and 25 nucleotides although other lengths are also acceptable. Typically the overhangs, if present, are 2 nucleotides in length, although they may be 1 nucleotide or longer than 2 nucleotides. In addition, it is assumed that the siRNA N19 inhibitory region will be chosen so that the core region of the antisense strand of the siRNA (i.e., the portion that is complementary to the target transcript) is perfectly complementary to the target transcript, though as mentioned above one or more mismatches may be tolerated. In general it is desirable to avoid mismatches in the duplex region if an siRNA having maximal ability to reduce expression of the target transcript via the classical pathway is desired. However, as described below, it may be desirable to select an siRNA that exhibits less than maximal ability to reduce expression of the target transcript, or it may be desirable to employ an siRNA that acts via the alternative pathway. In such situations it may be desirable to incorporate one or more mismatches in the duplex portion of the siRNA. In general, preferably fewer than four residues or alternatively less than about 15% of residues in the inhibitory region are mismatched with the target.

In some cases the siRNA sequence is selected such that the entire antisense strand (including the 3′ overhang if present) is perfectly complementary to the target transcript. In cases where the overhang is UU, TT, or dTdT, this requires that the 19 bp target region of the targeted transcript is preceded by AA (i.e., that the two nucleotides immediately 5′ of the target region are AA). Similarly, the siRNA sequence may be selected such that the entire sense strand (including the 3′ overhang) is perfectly identical to the target transcript. In cases where the overhang is UU, TT, or dTdT, this requires that the 19 bp target region of the targeted transcript is followed by UU (i.e., that the two nucleotides immediately 3′ of the target region of the target transcript are UU). However, it is not necessary that overhang(s) are either complementary or identical to the target transcript. Any desired sequence (e.g., UU) may simply be appended to the 3′ ends of antisense and/or sense 19 bp core regions of an siRNA to generate 3′ overhangs. In general, overhangs containing one or more pyrimidines, usually U, T, or dT, are employed. When synthesizing siRNAs it may be more convenient to use T rather than U, while use of dT rather than T may confer increased stability. As indicated above, the presence of overhangs is optional and, where present, they need not have any relationship to the target sequence itself. It is noted that since shRNAs have only one 3′ end, only a single 3′ overhang is possible prior to processing to form siRNA.

In summary, in general an siRNA may be designed by selecting any core region of appropriate length, e.g., 19 nt, in the target transcript, and selecting an siRNA having an antisense strand whose sequence is substantially or perfectly complementary to the core region and a sense strand whose sequence is complementary to the antisense strand of the siRNA. 3′ overhangs such as those described above may then be added to these sequences to generate an siRNA structure. Thus there is no requirement that the overhang in the antisense strand is complementary to the target transcript or that the overhang in the sense strand corresponds with sequence present in the target transcript. It will be appreciated that, in general, where the target transcript is an mRNA, siRNA sequences may be selected with reference to the corresponding sequence of double-stranded cDNA rather than to the mRNA sequence itself, since according to convention the sense strand of the cDNA is identical to the mRNA except that the cDNA contains T rather than U. (Note that in the context of the influenza virus replication cycle, double-stranded cDNA is not generated, and the cDNA present in the cell is single-stranded and is complementary to viral mRNA.)

Not all siRNAs are equally effective in reducing or inhibiting expression of any particular target gene. (See, e.g., Holen, T., et al., Nucleic Acids Res., 30(8):1757-1766, reporting variability in the efficacy of different siRNAs), and a variety of considerations may be employed to increase the likelihood that a selected siRNA proves to be effective. For example, it may be preferable to select target portions within exons rather than introns. In general, target portions near the 3′ end of a target transcript may be preferred to target portions near the 5′ end or middle of a target transcript. siRNAs may generally be designed in accordance with principles described in Technical Bulletin # 003-Revision B, “siRNA Oligonucleotides for RNAi Applications”, available from Dharmacon Research, Inc., Lafayette, Colo. 80026, a commercial supplier of RNA reagents. Technical Bulletins #003 (accessible on the World Wide Web at www.dharmacon.com/tech/tech003B.html) and #004 available at www.dharmacon.com/tech/tech004.html from Dharmacon contain a variety of information relevant to siRNA design parameters, synthesis, etc., and are incorporated herein by reference. Generally it is preferable to select siRNAs with a GC content between 30% and 60% and to avoid strings of three or more identical nucleotides, e.g., GGG, CCC, etc. In order to achieve specific inhibition of the target transcript while avoiding inhibition of other transcripts, it is desirable to select sequences that are unique or lack significant homology to other sequences present in the cell or organism to which the siRNA is delivered, to the extent possible. This may be achieved by searching publicly available databases, e.g., Genbank, draft human genome sequence, etc., to identify any sequences that are homologous to a proposed siRNA and avoiding the use of siRNAs for which identical homologous sequences are found.

One of ordinary skill in the art will appreciate that siRNAs may exhibit a range of melting temperatures (Tm) and dissociation temperatures (Td) in accordance with the foregoing principles. The Tm is defined as the temperature at which 50% of a nucleic acid and its perfect complement are in duplex in solution while the Td, defined as the temperature at a particular salt concentration, and total strand concentration at which 50% of an oligonucleotide and its perfect filter-bound complement are in duplex, relates to situations in which one molecule is immobilized on a filter. Representative examples of acceptable Tms may readily be determined using methods well known in the art, either experimentally or using appropriate empirically or theoretically derived equations, based on the effective siRNA sequences disclosed in the Examples herein.

One common way to determine the actual Tm is to use a thermostatted cell in a UV spectrophotometer. If temperature is plotted vs. absorbance, an S-shaped curve with two plateaus will be observed. The absorbance reading halfway between the plateaus corresponds to Tm. The simplest equation for Td is the Wallace rule: Td=2(A+T)+4(G+C) Wallace, R. B.; Shaffer, J.; Murphy, R. F.; Bonner, J.; Hirose, T.; Itakura, K., Nucleic Acids Res. 6, 3543 (1979). The nature of the immobilized target strand provides a net decrease in the Tm observed relative to the value when both target and probe are free in solution. The magnitude of the decrease is approximately 7-8° C. Another useful equation for DNA which is valid for sequences longer than 50 nucleotides from pH 5 to 9 within appropriate values for concentration of monovalent cations, is: Tm=81.5+16.6 log M+41 (XG+XC)−500/L−0.62F, where M is the molar concentration of monovalent cations, XG and XC are the mole fractions of G and C in the sequence, L is the length of the shortest strand in the duplex, and F is the molar concentration of formamide (Howley, P. M; Israel, M. F.; Law, M-F.; Martin, M. A., J. Biol. Chem. 254, 4876). Similar equations for RNA are: Tm=79.8+18.5 log M+58.4 (XG+XC)+11.8(XG+XC)2−820/L−0.35F and for DNA-RNA hybrids: Tₘ=79.8+18.5 log M+58.4 (XG+XC)+11.8(XG+XC)2−820/L−0.50F. These equations are derived for immobilized target hybrids. Several studies have derived accurate equations for Tm using thermodynamic basis sets for nearest neighbor interactions. The equation for DNA and RNA is: Tm=(1000ΔH)/A+ΔS+Rln(Ct/4)−273.15+16.6 ln[Na⁺], where ΔH (Kcal/mol) is the sum of the nearest neighbor enthalpy changes for hybrids, A (eu) is a constant containing corrections for helix initiation, ΔS (eu) is the sum of the nearest neighbor entropy changes, R is the Gas Constant (1.987 cal deg⁻¹ mol⁻¹) and Ct is the total molar concentration of strands. If the strand is self complementary, Ct/4 is replaced by Ct. Values for thermodynamic parameters are available in the literature. For DNA see Breslauer, et al., Proc. Natl. Acad. Sci. USA 83, 3746-3750, 1986. For RNA:DNA duplexes see Sugimoto, N., et al, Biochemistry, 34(35): 11211-6, 1995. For RNA see Freier, S. M., et al., Proc. Natl. Acad. Sci. 83, 9373-9377, 1986. Rychlik, W., et al., Nucl. Acids Res. 18(21), 6409-6412, 1990. Various computer programs for calculating Tm are widely available. See, e.g., the Web site having URL www.basic.nwu.edu/biotools/oligocalc.html. According to certain embodiments of the invention, preferred siRNAs are selected in accordance with the design criteria described in Semizarov, D., et al., Proc. Natl. Acad. Sci., 100(11), pp. 6347-6352.

Certain siRNAs hybridize to a target site that includes one or more 3′ UTR sequences. In fact, in certain embodiments of the invention, the siRNA hybridizes completely within the 3′ UTR. Such embodiments of the invention may tolerate a larger number of mismatches in the siRNA/template duplex, and particularly may tolerate mismatches within the central region of the duplex. In fact, some mismatches may be desirable as siRNA/template duplex formation in the 3′ UTR may inhibit expression of a protein encoded by the template transcript by a mechanism related to but distinct from classic RNA inhibition. In particular, there is evidence to suggest that siRNAs that bind to the 3′ UTR of a template transcript may reduce translation of the transcript rather than decreasing its stability. For example, when hybridized with the target transcript such siRNAs frequently include two stretches of perfect complementarity separated by a region of mismatch. A variety of structures are possible. For example, the siRNA may include multiple areas of nonidentity (mismatch). The areas of nonidentity (mismatch) need not be symmetrical in the sense that both the target and the siRNA include nonpaired nucleotides. Typically the stretches of perfect complementarity are at least 5 nucleotides in length, e.g., 6, 7, or more nucleotides in length, while the regions of mismatch may be, for example, 1, 2, 3, or 4 nucleotides in length.

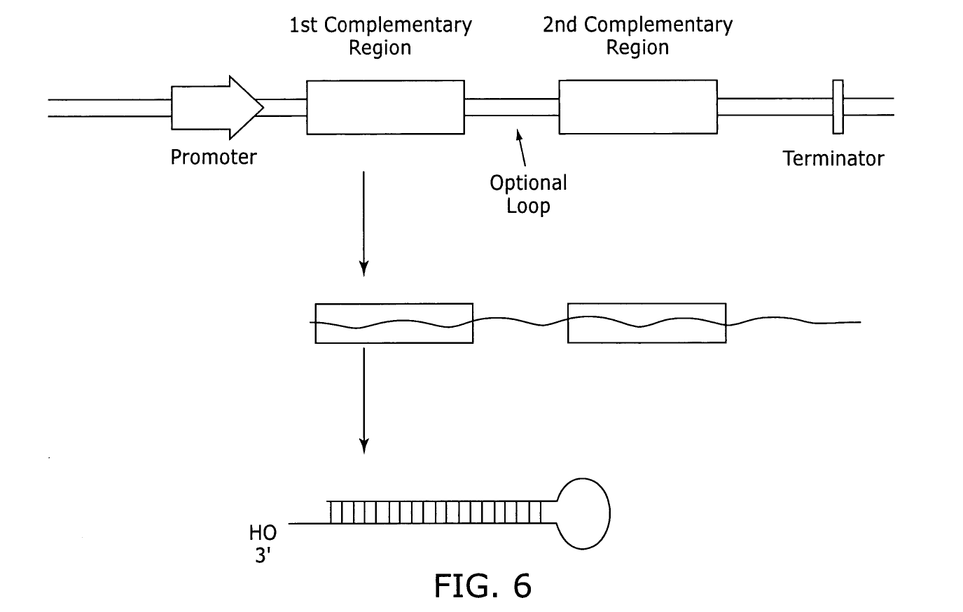

Certain siRNAs hybridize to a target site that includes or consists entirely of 3′ UTR sequences. Such siRNAs may tolerate a larger number of mismatches in the siRNA/template duplex, and particularly may tolerate mismatches within the central region of the duplex. For example, one or both of the strands may include one or more “extra” nucleotides that form a bulge as shown in FIG. 6. Typically the stretches of perfect complementarity are at least 5 nucleotides in length, e.g., 6, 7, or more nucleotides in length, while the regions of mismatch may be, for example, 1, 2, 3, or 4 nucleotides in length. When hybridized with the target transcript such siRNAs frequently include two stretches of perfect complementarity separated by a region of mismatch. A variety of structures are possible. For example, the siRNA may include multiple areas of nonidentity (mismatch). The areas of nonidentity (mismatch) need not be symmetrical, i.e., it is not required that both the target and the siRNA include nonpaired nucleotides.

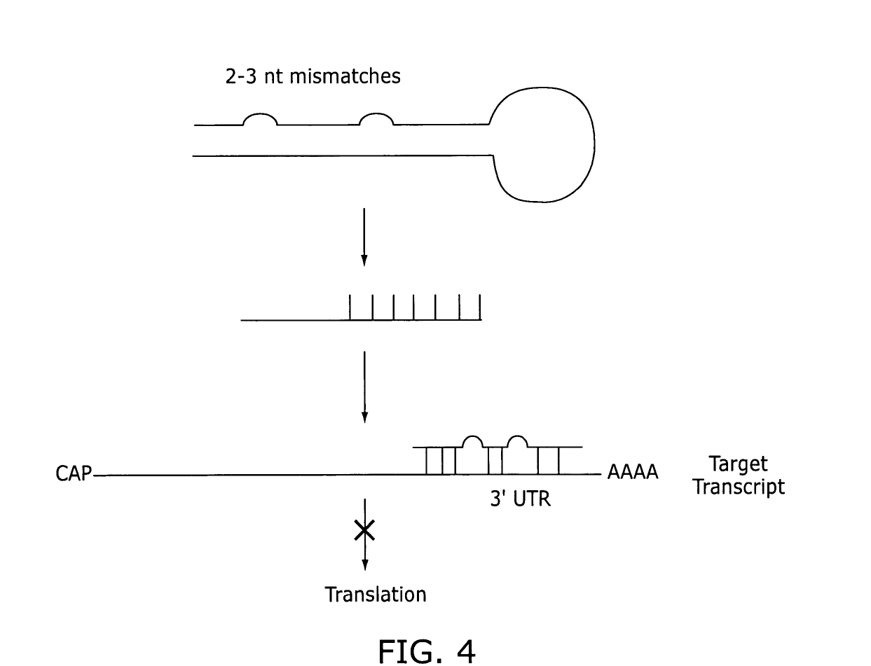

Some mismatches may be desirable, as siRNA/template duplex formation in the 3′ UTR may inhibit expression of a protein encoded by the template transcript by a mechanism related to but distinct from classic RNA inhibition. In particular, there is evidence to suggest that siRNAs that bind to the 3′ UTR of a template transcript may reduce translation of the transcript rather than decreasing its stability. Specifically, as shown in FIG. 4, the DICER enzyme that generates siRNAs in the Drosophila system discussed above and also in a variety of organisms, is known to also be able to process a small, temporal RNA (stRNA) substrate into an inhibitory agent that, when bound within the 3′ UTR of a target transcript, blocks translation of the transcript (see Grishok, A., et al., Cell 106, 23-24, 2001; Hutvagner, G., et al., Science, 293, 834-838, 2001; Ketting, R., et al., Genes Dev., 15, 2654-2659). For the purposes of the present invention, any partly or fully double-stranded short RNA as described herein, one strand of which binds to a target transcript and reduces its expression (i.e., reduces the level of the transcript and/or reduces synthesis of the polypeptide encoded by the transcript) is considered to be an siRNA, regardless of whether the RNA acts by triggering degradation, by inhibiting translation, or by other means. In certain preferred embodiments of the invention, reducing expression of the transcript involves degradation of the transcript. In addition any precursor structure (e.g., a short hairpin RNA, as described herein) that may be processed in vivo (i.e., within a cell or organism) to generate such an siRNA is useful in the practice of the present invention.

Those of ordinary skill in the art will readily appreciate that inventive RNAi-inducing agents may be prepared according to any available technique including, but not limited to chemical synthesis, enzymatic or chemical cleavage in vivo or in vitro, or template transcription in vivo or in vitro. As noted above, inventive RNA-inducing agents may be delivered as a single RNA molecule including self-complementary portions (i.e., an shRNA that can be processed intracellularly to yield an siRNA), or as two strands hybridized to one another. For instance, two separate 21 nt RNA strands may be generated, each of which contains a 19 nt region complementary to the other, and the individual strands may be hybridized together to generate a structure such as that depicted in FIG. 3A.

Alternatively, each strand may be generated by transcription from a promoter, either in vitro or in vivo. For instance, a construct may be provided containing two separate transcribable regions, each of which generates a 21 nt transcript containing a 19 nt region complementary with the other. Alternatively, a single construct may be utilized that contains opposing promoters P1 and P2 and terminators t1 and t2 positioned so that two different transcripts, each of which is at least partly complementary to the other, are generated is indicated in FIG. 5.

In another embodiment, an RNA-inducing entity is generated as a single transcript, for example by transcription of a single transcription unit comprising self complementary regions. FIG. 6 depicts one such embodiment of the present invention. As indicated, a template is employed that includes first and second complementary regions, and optionally includes a loop region. Such a template may be utilized for in vitro or in vivo transcription, with appropriate selection of promoter (and optionally other regulatory elements). The present invention encompasses use of constructs capable of serving as templates for transcription of one or more siRNA strands.

In vitro transcription may be performed using a variety of available systems including the T7, SP6, and T3 promoter/polymerase systems (e.g., those available commercially from Promega, Clontech, New England Biolabs, etc.). As will be appreciated by one of ordinary skill in the art, use of the T7 or T3 promoters typically requires an siRNA sequence having two G residues at the 5′ end while use of the SP6 promoter typically requires an siRNA sequence having a GA sequence at its 5′ end. Vectors including the T7, SP6, or T3 promoter are well known in the art and can readily be modified to direct transcription of siRNAs. When siRNAs or shRNAs are synthesized in vitro they may be allowed to hybridize before transfection or delivery to a subject. It is to be understood that inventive siRNA compositions need not consist entirely of double-stranded (hybridized) molecules. For example, siRNA compositions may include a small proportion of single-stranded RNA. This may occur, for example, as a result of the equilibrium between hybridized and unhybridized molecules, because of unequal ratios of sense and antisense RNA strands, because of transcriptional termination prior to synthesis of both portions of a self-complementary RNA, etc. Generally, preferred compositions comprise at least approximately 80% double-stranded RNA, at least approximately 90% double-stranded RNA, at least approximately 95% double-stranded RNA, or even at least approximately 99-100% double-stranded RNA. However, the siRNA compositions may contain less than 80% hybidized RNA provided that they contain sufficient double-stranded RNA to be effective.

Those of ordinary skill in the art will appreciate that, where inventive siRNA agents are to be generated in vivo, it is generally preferable that they be produced via transcription of one or more transcription units. The primary transcript may optionally be processed (e.g., by one or more cellular enzymes) in order to generate the final agent that accomplishes gene inhibition. It will further be appreciated that appropriate promoter and/or regulatory elements can readily be selected to allow expression of the relevant transcription units in mammalian cells. In some embodiments of the invention, it may be desirable to utilize a regulatable promoter; in other embodiments, constitutive expression may be desired. It is noted that the term “expression” as used herein in reference to synthesis (transcription) of siRNA or siRNA precursors does not imply translation of the transcribed RNA.

In certain preferred embodiments of the invention, the promoter utilized to direct in vivo expression of one or more siRNA or shRNA transcription units is a promoter for RNA polymerase III (Pol III). Pol III directs synthesis of small transcripts that terminate within a stretch of 4-5 T residues. Certain Pol III promoters such as the U6 or H1 promoters do not require cis-acting regulatory elements (other than the first transcribed nucleotide) within the transcribed region and thus are preferred according to certain embodiments of the invention since they readily permit the selection of desired siRNA sequences. In the case of naturally occurring U6 promoters the first transcribed nucleotide is guanosine, while in the case of naturally occurring H1 promoters the first transcribed nucleotide is adenine. (See, e.g., Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Tuschl, T., et al., Nat. Biotech., 20, 446-448 (2002). Thus in certain embodiments of the invention, e.g., where transcription is driven by a U6 promoter, the 5-nucleotide of preferred siRNA sequences is G. In certain other embodiments of the invention, e.g., where transcription is driven by an H1 promoter, the 5′ nucleotide may be A.

According to certain embodiments of the invention promoters for RNA polymerase II (Pol II) may also be used as described, for example, in Xia, H., et al., Nat. Biotechnol., 20, pp. 1006-1010, 2002. As described therein, constructs in which a hairpin sequence is juxtaposed within close proximity to a transcription start site and followed by a polyA cassette, resulting in minimal to no overhangs in the transcribed hairpin, may be employed. In certain embodiments of the invention tissue-specific, cell-specific, or inducible Pol II promoters may be used, provided the foregoing requirements are met. For example, it may be desirable to use mast cell specific, T cell specific, or B cell specific promoters. In addition, in certain embodiments of the invention promoters for Pol I may be used as described, for example, in (McCown 2003).

It will be appreciated that in vivo expression of constructs that provide templates for synthesis of siRNA or shRNA, such as those depicted in FIGS. 5 and 6, can desirably be accomplished by introducing the constructs into a vector, such as, for example, a DNA plasmid or viral vector, and introducing the vector into mammalian cells. Any of a variety of vectors may be selected, though in certain embodiments of the invention it is desirable to select a vector that can deliver the construct(s) to one or more cells in the respiratory passages. The present invention encompasses compositions comprising vectors containing siRNA or shRNA transcription units. In certain preferred embodiments of the invention, the vectors are gene therapy vectors appropriate for the delivery of the construct to mammalian cells (e.g., cells of a domesticated mammal), and most preferably human cells.

A variety of different siRNA-inducing vectors may be used in the compositions of the invention. In certain embodiments of the invention two separate, complementary siRNA strands are transcribed using a single vector containing two promoters, each of which directs transcription of a single siRNA strand, i.e., the promoter is operably linked to a template for the siRNA strand so that transcription occurs. In certain embodiments of the invention two separate, complementary siRNA strands are transcribed using a single vector containing two promoters, each of which directs transcription of a single siRNA strand, i.e., is operably linked to a template for the siRNA so that transcription occurs. The two promoters may be in the same orientation, in which case each is operably linked to a template for one of the siRNA strands. Alternately, the promoters may be in opposite orientation flanking a single template so that transcription from the promoters results in synthesis of two complementary RNA strands.

In other embodiments of the invention a vector containing a promoter that drives transcription of a single RNA molecule comprising two complementary regions (e.g., an shRNA) is employed. In certain embodiments of the invention a vector containing multiple promoters, each of which drives transcription of a single RNA molecule comprising two complementary regions is used. Alternately, multiple different shRNAs may be transcribed, either from a single promoter or from multiple promoters. A variety of configurations are possible. For example, a single promoter may direct synthesis of a single RNA transcript containing multiple self-complementary regions, each of which may hybridize to generate a plurality of stem-loop structures. These structures may be cleaved in vivo, e.g., by DICER, to generate multiple different shRNAs. It will be appreciated that such transcripts preferably contain a termination signal at the 3′ end of the transcript but not between the individual shRNA units. It will also be appreciated that single RNAs from which multiple siRNAs can be generated need not be produced in vivo but may instead be chemically synthesized or produced using in vitro transcription and provided exogenously. In another embodiment of the invention, the vector includes multiple promoters, each of which directs synthesis of a self-complementary RNA that hybridizes to form an siRNA. The multiple siRNAs may all target the same transcript, or they may target different transcripts. Any combination of transcripts may be targeted.

In another embodiment of the invention, the vector includes multiple promoters, each of which directs synthesis of a self-complementary RNA molecule that hybridizes to form an shRNA. The multiple shRNAs may all target the same transcript, or they may target different transcripts. Any combination of transcripts may be targeted.

Those of ordinary skill in the art will further appreciate that in vivo expression of siRNAs or shRNAs from RNAi-inducing vectors delivered according to the present invention may allow the production of cells that produce the siRNA or shRNA over long periods of time (e.g., greater than a few days, preferably at least several weeks to months, more preferably at least a year or longer, possibly a lifetime).

Retroviral vectors, e.g., lentiviral vectors, whose presence within a cell results in transcription of one or more RNAs that self-hybridize or hybridize to each other to form an shRNA or siRNA that inhibits expression of at least one transcript in the cell may be used in the compositions of the invention. For purposes of description it will be assumed that the vector is a lentiviral vector such as those described in Rubinson, D., et al, Nature Genetics, Vol. 33, pp. 401-406, 2003. However, it is to be understood that other retroviral or lentiviral vectors may also be used. According to various embodiments of the invention the lentiviral vector may be either a lentiviral transfer plasmid or a lentiviral particle, e.g., a lentivirus capable of infecting cells. In certain embodiments of the invention the lentiviral vector comprises a nucleic acid segment operably linked to a promoter, so that transcription from the promoter (i.e., transcription directed by the promoter) results in synthesis of an RNA comprising complementary regions that hybridize to form an shRNA targeted to the target transcript. According to certain embodiments of the invention the shRNA comprises a base-paired region approximately 19 nucleotides long. According to certain embodiments of the invention the RNA may comprise more than 2 complementary regions, so that self-hybridization results in multiple base-paired regions, separated by loops or single-stranded regions. The base-paired regions may have identical or different sequences and thus may be targeted to the same or different regions of a single transcript or to different transcripts.

In certain embodiments of the invention the lentiviral vector comprises a nucleic acid segment flanked by two promoters in opposite orientation, wherein the promoters are operably linked to the nucleic acid segment, so that transcription from the promoters results in synthesis of two complementary RNAs that hybridize with each other to form an siRNA targeted to the target transcript. According to certain embodiments of the invention the siRNA comprises a base-paired region approximately 19 nucleotides long. In certain embodiments of the invention the lentiviral vector comprises at least two promoters and at least two nucleic acid segments, wherein each promoter is operably linked to a nucleic acid segment, so that transcription from the promoters results in synthesis of two complementary RNAs that hybridize with each other to form an siRNA targeted to the target transcript.

As mentioned above, the lentiviral vectors for use in the compositions of the invention may be lentiviral transfer plasmids or lentiviral particles (e.g., a lentivirus or pseudotyped lentivirus). See, e.g., U.S. Pat. No. 6,013,516 and references 113-117 for further discussion of lentiviral transfer plasmids, lentiviral particles, and lentiviral expression systems. As is well known in the art, lentiviruses have an RNA genome. Therefore, where the lentiviral vector is a lentiviral particle, e.g., an infectious lentivirus, the viral genome must undergo reverse transcription and second strand synthesis to produce DNA capable of directing RNA transcription. In addition, where reference is made herein to elements such as promoters, regulatory elements, etc., it is to be understood that the sequences of these elements are present in RNA form in the lentiviral particles and are present in DNA form in the lentiviral plasmids. Furthermore, where a template for synthesis of an RNA is “provided by” RNA present in a lentiviral particle, it is understood that the RNA must undergo reverse transcription and second strand synthesis to produce DNA that can serve as a template for synthesis of RNA (transcription). Vectors that provide templates for synthesis of siRNA or shRNA are considered to provide the siRNA or shRNA when introduced into cells in which such synthesis occurs.

III. Compositions and Methods for Improved Delivery of RNAi-inducing Entities

Effective use of RNAi in humans and other mammals for such purposes as prevention and therapy of infections and other diseases and conditions will be enhanced by efficient delivery of RNAi-inducing entities to cells in which inhibition of a transcript is desired. For use in humans, it may be preferable to employ non-viral methods that facilitate intracellular uptake of RNAi-inducing entities such as siRNA, shRNA, or RNAi-inducing vectors (e.g., DNA vectors). The invention therefore provides compositions comprising any of a variety of non-viral delivery agents for enhanced delivery of siRNA, shRNA, and/or RNAi-inducing vectors to cells. While it is anticipated that the delivery agents described herein will primarily be used to enhance delivery of RNA or DNA rather than intact virus, their use for the latter purpose is not excluded.

As used herein, the concept of “delivery” includes transport of an RNAi-inducing entity such as an siRNA, shRNA, or RNAi-inducing vector from its site of entry into the body to the location of the cells in which it is to function, in addition to cellular uptake of the entity and any subsequent steps involved in making siRNA or shRNA available to the intracellular RNAi machinery (e.g., release or siRNA or shRNA from endosomes). In general, the delivery agents described herein serve as a vehicle or carrier for delivery of the RNA or vector, facilitate one or more steps in the process of making the siRNA or shRNA available to the intracellular RNAi machinery, and/or help to protect or stabilize the RNAi-inducing entity within the body.

The invention therefore provides compositions comprising an one or more RNAi-inducing entity targeted to a transcript and any of a variety of delivery agents including, but not limited to, cationic polymers, modified cationic polymers, peptide molecular transporters (including arginine or histidine-rich peptides), lipids (including cationic lipids, neutral lipids, and combinations thereof), liposomes, lipopolyplexes, non-cationic polymers, modified non-cationic polymers, chloroquine, bupivacaine, and surfactants suitable for introduction into the lung. Certain of the delivery agents are modified to incorporate a moiety that increases delivery or increases the selective delivery of the one or more RNAi-inducing entity to cells in which it is desired to inhibit a particular transcript.

In certain embodiments of the invention a disease or condition, or a symptom thereof, is associated with, characterized by, or features inappropriate or excessive expression of the transcript or inappropriate or excessive functional activity of a polypeptide encoded by the transcript. In certain preferred embodiments of the invention adminstration of the composition inhibits expression of the transcript, thereby treating the disease. The invention therefore provides a method of treating a disease or condition, or a symptom thereof, is associated with, characterized by, or features inappropriate or excessive expression of the transcript or inappropriate or excessive functional activity of a polypeptide encoded by the transcript, comprising the step of administering a composition comprising an RNAi-inducing entity, wherein the RNAi-inducing entity is targeted to the transcript and a delivery agent selected from the group consisting of cationic polymers, modified cationic polymers, peptide molecular transporters (including arginine or histidine-rich peptides), lipids (including cationic lipids, neutral lipids, and combinations thereof), liposomes, lipopolyplexes, non-cationic polymers, modified non-cationic polymers, chloroquine, bupivacaine, and surfactants suitable for introduction into the lung. In various embodiments of the invention the RNAi-inducing entity is an siRNA, shRNA, or RNAi-inducing vector.

In certain preferred embodiments of the invention the compositions provide enhanced delivery of RNAi-inducing entities to the lung. However, the efficacy of the various delivery agents described herein is not limited to particular cell types. Therefore, various embodiments of the invention encompass delivery of RNAi-inducing entities to any cell, tissue or solid organ (e.g., lung, liver, heart, kidney, spleen, pancreas, intestine, bladder, thymus, endocrine glands, breast, uterus, testes, skin etc.) in the body. Various embodiments of the invention also encompass delivery of RNAi-inducing entities such as siRNA, shRNA, or RNAi-inducing vectors to cells in the walls of blood vessels, e.g., endothelial cells, smooth muscle cells, fibroblasts, macrophages, etc., and to cells in the blood itself, e.g., lymphocytes, neutrophils, etc.

A. Cationic Polymers and Modified Cationic Polymers

Cationic polymer-based systems have been investigated as carriers for DNA transfection (35). The ability of cationic polymers to promote cellular uptake of DNA is thought to arise partly from their ability to bind to DNA and condense large plasmid DNA molecules into smaller DNA/polymer complexes for more efficient endocytosis. The DNA/cationic polymer complexes also act as bioadhesives because of their electrostatic interaction with negatively charged sialic acid residues of cell surface glycoproteins (36). In addition, some cationic polymers apparently promote disruption of the endosomal membrane and therefore release of DNA into the cytosol (32). The invention therefore provides a composition comprising (i) an RNAi-inducing entity, wherein the RNAi-inducing entity is targeted to a target transcript; and (ii) a cationic polymer. The invention further provides methods of inhibiting target transcripts by administering such compositions.

In general, a cationic polymer is a polymer that is positively charged at approximately physiological pH, e.g., a pH ranging from approximately 7.0 to 7.6, preferably approximately 7.2 to 7.6, more preferably approximately 7.4. Such cationic polymers include, but are not limited to, polylysine (PLL), polyarginine (PLA), polyhistidine, polyethyleneimine (PEI) (37), including linear PEI and low molecular weight PEI as described, for example, in (76), polyvinylpyrrolidone (PVP) (38), and chitosan (39, 40). It will be appreciated that certain of these polymers comprise primary amine groups, imine groups, guanidine groups, and/or imidazole groups. Preferred cationic polymers have relatively low toxicity and high DNA transfection efficiency.

Suitable cationic polymers also include copolymers comprising subunits of any of the foregoing polymers, e.g., lysine-histidine copolymers, etc. The percentage of the various subunits need not be equal in the copolymers but may be selected, e.g., to optimize such properties as ability to form complexes with nucleic acids while minimizing cytotoxicity. Furthermore, the subunits need not alternate in a regular fashion. Appropriate assays to evaluate various polymers with respect to desirable properties are described in the Examples. Preferred cationic polymers also include polymers such as the foregoing, further incorporating any of various modifications. Appropriate modifications are discussed below and include, but are not limited to, modification with acetyl, succinyl, acyl, or imidazole groups (32).

While not wishing to be bound by any theory, it is believed that cationic polymers such as PEI compact or condense DNA into positively charged particles capable of interacting with anionic proteoglycans at the cell surface and entering cells by endocytosis. Such polymers may possess the property of acting as a “proton sponge” that buffers the endosomal pH and protects DNA from degradation. Continuous proton influx also induces endosome osmotic swelling and rupture, which provides an escape mechanism for DNA particles to the cytoplasm. The inventors have recognized that similar considerations apply to the delivery of siRNA, shRNA, and DNA vectors that provide templates for synthesis of siRNA or shRNA. Furthermore, the inventors have demonstrated effective delivery of siRNA and DNA vectors providing a template for synthesis of shRNA to cells in mammalian subjects, resulting in inhibition of target transcripts using a variety of such agents. References 85-87; U.S. Pat. No. 6,013,240; WO9602655 provide further information on PEI and other cationic polymers useful in the practice of the invention. According to certain embodiments of the invention the commercially available PEI reagent known as jetPEI™ (Qbiogene, Carlsbad, Calif.), a linear form of PEI (U.S. Pat. No. 6,013,240) is used.

As described in Example 4, the inventors have shown that compositions comprising PEI, PLL, or PLA and an siRNA that targets an influenza virus RNA significantly inhibit production of influenza virus in mice when administered intravenously either before or after influenza virus infection. The inhibition is dose-dependent and exhibits additive effects when two siRNAs targeted to different influenza virus RNAs were used. Thus siRNA, when combined with a cationic polymer and introduced into the vascular system, is able to reach the lung, to enter cells, and to effectively inhibit the viral replication cycle. Furthermore, in the absence of the cationic polymer (i.e., when siRNA was administered intravenously in phosphate buffered saline), no such inhibition was observed. It is believed that these findings represent the first report of enhanced efficacy of siRNA in mammals by admininstration of the siRNA in combination with a delivery agent other than a standard buffer solution. It is also believed that these findings represent the first report of efficacy in inhibiting a complete viral life cycle (i.e., a sequence of events beginning with infection by an intact virus and leading to production of infectious virus in the host) in a mammalian subject using siRNA. It is noted that the experiments described herein involved immunocompetent animals, thus providing a realistic model for the efficacy of RNAi in inhibiting viral infection in a clinically relevant setting. Since RNAi has been proven to effectively inhibit virtually any target transcript in a mammalian cell, the demonstrations of effiacy in inhibiting influenza virus transcripts described herein are equally relevant for the inhibition of endogenous cellular transcripts or transcripts of other infectious agents present in mammalian cells.

The invention therefore provides a method of inhibiting expression of a target transcript in a cell within a mammalian subject comprising the step of introducing a composition comprising an RNAi-inducing entity targeted to the target transcript and a cationic polymer into the vascular system of the subject. In certain embodiments of the invention the RNAi-including entity is an siRNA, shRNA, or RNAi-inducing vector. In certain preferred embodiments of the invention the composition is introduced into a vein, e.g., by intravenous injection. However, the composition may also be administered into an artery, delivered using a device such as a catheter, indwelling intravenous line, etc. In certain embodiments of the invention the target transcript is expressed in the lung, and expression of the target transcript is inhibited in the lung. However, the invention is not limited to delivery of RNAi-inducing entities to the lung since the ability of cationic polymers and other agents described herein to enhance cellular delivery is not limited to particular cell types.

It is noted that other efforts to deliver RNAi-inducing entities intravenously to solid organs and tissues within the body (see, e.g., McCaffrey 2002; McCaffrey 2003; Lewis, D. L., et al.) have employed the technique known as hydrodynamic transfection, which involves rapid delivery of large volumes of fluid into the tail vein of mice and has been shown to result in accumulation of significant amounts of plasmid DNA in solid organs, particularly the liver (Liu 1999; Zhang 1999; Zhang 2000). This technique involves delivery of fluid volumes that are almost equivalent to the total blood volume of the animal, e.g., 1.6 ml for mice with a body weight of 18-20 grams, equivalent to approximately 8-12% of body weight, as opposed to conventional techniques that involve injection of approximately 200 μl of fluid (Liu 1999). In addition, injection using the hydrodynamic transfection approach takes place over a short time interval (e.g., 5 seconds), which is necessary for efficient expression of injected transgenes (Liu 1999).

While the mechanism by which hydrodynamic transfection achieves transfer and high level expression of injected transgenes in the liver is not entirely clear, it is thought to be due to a reflux of DNA solution into the liver via the hepatic vein due to a transient cardiac congestion (Zhang 2000). A comparable approach for therapeutic purposes in humans seems unlikely to be feasible. The inventors, in contrast, have used conventional volumes of fluid (e.g., 200 μl) and have demonstrated effective delivery of siRNA to the lung under conditions that would be expected to lead to minimal expression of injected transgenes even in the liver, the site at which expression is most readily achieved using hydrodynamic transfection. The invention thus provides a method of inhibiting expression of a target transcript in a mammalian cell within a subject comprising the step of introducing a composition comprising an RNAi-inducing entity targeted to the target transcript into the vascular system of the subject using a conventional delivery technique, e.g., a technique using conventional pressures and/or conventional volumes of fluid. In preferred embodiments of the invention the composition is introduced in a fluid volume equivalent to less than 10% of the subject's body weight. In certain embodiments of the invention the fluid volume is equivalent to less than 5%, less than 2%, less than 1%, or less than 0.1% of the subject's body weight. In certain embodiments of the invention application of the method results in effective amounts of siRNA or shRNA in a cell in a body tissue or organ other than the liver. In certain embodiments of the invention the amount of siRNA or shRNA resulting in the cell is sufficient to inhibit production of an infectious agent such as a virus or parasite. In certain preferred embodiments of the invention the composition inhibits a target transcript in the lung, e.g., in respiratory epithelial cells.

In certain embodiments of the invention the compositions may be used to enhance delivery of RNAi-inducing entities to non-mammalian cells within the body of a mammalian subject, e.g., cells of a parasite. The invention therefore provides a method of inhibiting expression of a target transcript in a non-mammalian cell within a mammalian subject comprising the step of introducing a composition comprising an RNAi-inducing entity targeted to the target transcript and a cationic polymer into the vascular system of the subject. In certain embodiments of the invention the amount of siRNA or shRNA resulting from delivery of the composition is sufficient to inhibit production of an infectious agent such as a virus or parasite within the subject.

As described in Example 7, the inventors have also shown that the cationic polymers PLL and PLA are able to form complexes with siRNAs and promote uptake of functional siRNA in cultured cells. Transfection with complexes of PLL and NP-1496 or complexes of PLA and NP-1496 siRNA inhibited production of influenza virus in cells. These results and the results in mice discussed above demonstrate the feasibility of using mixtures of cationic polymers and siRNA for delivery of siRNA to mammalian cells in the body of a subject. The approach described in Example 7 may be employed to test additional polymers, particularly polymers modified by addition of groups (e.g., acyl, succinyl, acetyl, or imidazole groups) to reduce cytotoxicity, and to optimize those that are initially effective. In general, certain preferred modifications result in a reduction in the positive charge of the cationic polymer. Certain preferred modifications convert a primary amine into a secondary amine. Methods for modifying cationic polymers to incorporate such additional groups are well known in the art. (See, e.g., reference 32). For example, the ε-amino group of various residues may be substituted, e.g., by conjugation with a desired modifying group after synthesis of the polymer. In general, it is desirable to select a % substitution sufficient to achieve an appropriate reduction in cytotoxicity relative to the unsubstituted polymer while not causing too great a reduction in the ability of the polymer to enhance delivery of the RNAi-inducing entity. Accordingly, in certain embodiments of the invention between 25% and 75% of the residues in the polymer are substituted. In certain embodiments of the invention approximately 50% of the residues in the polymer are substituted. It is noted that similar effects may be achieved by initially forming copolymers of appropriately selected monomeric subunits, i.e., subunits some of which already incorporate the desired modification.

A variety of additional cationic polymers may also be used. Large libraries of novel cationic polymers and oligomers from diacrylate and amine monomers have been developed and tested in DNA transfection. These polymers are referred to herein as poly(β-amino ester) (PAE) polymers. For example, a library of 140 polymers from 7 diacrylate monomers and 20 amine monomers has been described (34) and larger libraries can be produced using similar or identical methodology. Of the 140 members of this library, 70 were found sufficiently water-soluble (2 mg/ml, 25 mM acetate buffer, pH=5.0). Fifty-six of the 70 water-soluble polymers interacted with DNA as shown by electrophoretic mobility shift. Most importantly, two of the 56 polymers mediated DNA transfection into COS-7 cells. Transfection efficiencies of the novel polymers were 4-8 times higher than PEI and equal or better than Lipofectamine 2000. The invention therefore provides compositions comprising at least one siRNA molecule and a cationic polymer, wherein the cationic polymer is a poly(β-amino ester), and methods of inhibiting target gene expression by administering such compositions. Poly(beta-amino esters) are further described in U.S. published patent application 20020131951, entitled “Biodegradable poly(beta-amino esters) and uses thereof”, filed Sep. 19, 2002, by Langer et al. It is noted that the cationic polymers for use to facilitate delivery of RNAi-inducing entities may be modified so that they incorporate one or more residues other than the major monomeric subunit of which the polymer is comprised. For example, one or more alternate residues may be added to the end of a polymer, or polymers may be joined by a residue other than the major monomer of which the polymer is comprised.

Additional cationic polymers that may also be used to enhance delivery of RNAi-inducing entities include polyamidoamine (PAMAM) dendrimers, poly(2-dimethylamino)ethyl methacrylate (pDMAEMA), and its quaternary amine analog poly(2-triemethylamino)ethyl methacrylate (pTMAEMA), poly [a-(4-aminobutyl)-L-glycolic acid (PAGA), and poly (4-hydroxy-1-proline ester). See Han (2000) for further description of these agents.

B. Peptide Molecular Transporters

Studies have shown that a variety of peptides are able to act as delivery agents for nucleic acids. (As used herein, a polypeptide is considered to be a “peptide” if it shorter than approximately 50 amino acids in length.) For example, transcription factors, including HIV Tat protein (42, 43), VP22 protein of herpes simplex virus (44), and Antennapedia protein of Drosophila (45), can penetrate the plasma membrane from the cell surface. The peptide segments responsible for membrane penetration consist of 11-34 amino acid residues, are highly enriched for arginine, and are often referred to as arginine rich peptides (ARPs) or penetratins. When covalently linked with much larger polypeptides, the ARPs are capable of transporting the fused polypeptide across the plasma membrane (46-48). Similarly, when oligonucleotides were covalently linked to ARPs, they were much more rapidly taken up by cells (49, 50). Recent studies have shown that a polymer of eight arginines is sufficient for this transmembrane transport (51). Like cationic polymers, ARPs are also positively charged and likely capable of binding RNA, suggesting that it is probably not necessary to covalently link siRNA or shRNA to ARPs.

The invention therefore provides compositions comprising at least one RNAi-inducing entity, wherein the RNAi-inducing entity is targeted to a transcript, and a peptide molecular transporter and methods of inhibiting target transcript expression by administering such compositions. Peptide molecular transporters include, but are not limited to, those described in references 46-51, 120, and 134-136 and variations thereof evident to one of ordinary skill in the art. Arginine-rich peptides include a peptide consisting of arginine residues only.

Generally, preferred peptide molecular transporters are less than approximately 50 amino acids in length. According to certain embodiments of the invention the peptide molecular transporter is a peptide having length between approximately 7 and 34 amino acids. Many of the preferred peptides are arginine-rich. According to certain embodiments of the invention a peptide is arginine-rich if it includes at least 20%, at least 30%, or at least 40%, or at least 50%, or at least 60% or at least 70%, or at least 80%, or at least 90% arginine. According to certain embodiments of the invention the peptide molecular transporter is an arginine-rich peptide that includes between 6 and 20 arginine residues. According to certain embodiments of the invention the arginine-rich peptide consists of between 6 and 20 arginine residues. According to certain embodiments of the invention the RNAi-inducing entity and the peptide molecular transporter are covalently bound, whereas in other embodiments of the invention the RNAi-inducing entity and the peptide molecular transporter are mixed together but are not covalently bound to one another. According to certain embodiments of the invention a histidine-rich peptide is used (88). In accordance with the invention histidine-rich peptides may exhibit lengths and percentage of histidine residues as described for arginine-rich peptides. The invention therefore provides compositions comprising at least one RNAi-inducing entity targeted to a target transcript and a histidine-rich peptide and methods of inhibiting target transcript expression by administering such compositions.

Additional peptides or modified peptides that facilititate the delivery of RNAi-inducing entities to cells in a subject may also be used in the inventive compositions. For example, a family of lysine-rich peptides has been described, generally containing between 8 and approximately 50 lysine residues (McKenzie 2000). While these peptides can enhance uptake of nucleic acids by cells in tissue culture, they are less efficient delivery vehicles for nucleic acids in the body of a subject than longer polypeptides, e.g., PLL comprising more than 50 lysine residues. This may be due in part to insufficient stability of the nucleic acid/peptide complex within the body. Insertion of multiple cysteines at various positions within the peptides results in low molecular weight DNA condensing peptides that spontaneously oxidize after binding plasmid DNA to form interpeptide disulfide bonds. These cross-linked DNA delivery vehicles were more efficient inducers of gene expression when used to deliver plasmids to cells relative to uncrosslinked peptide DNA condensates (McKenzie 2002). In addition, peptides that comprise sulfhydryl residues for formation of disulfide bonds may incorporate polyethylene glycol (PEG), which is believed to reduce nonspecific binding to serum proteins (Park 2002).

Glycopeptides that include moieties such as galactose or mannose residues may also be used to enhance the selective uptake of RNAi-inducing entities in accordance with the present invention, as discussed further below. Such glycopeptides may also include sulfhydryl groups for formation of disulfide bonds (Park 2002). The invention encompasses administration of various agents that enhance exit of nucleic acids from endocytic vesicles. Such agents include chloroquine (Zhang 2003) and bupivacaine (Satishchandran 2000). The exit-enhancing agents may be administered systemically, orally, and/or locally (e.g. at or in close proximity to the desired site of action). They may be delivered together with RNAi-inducing entities or separately.

The invention encompasses modification of the other delivery agents described herein (e.g., polymeric delivery agents) to incorporate a peptide molecular transporter to facilitate transport of the delivery agent into cells.

C. Additional Polymeric Delivery Agents

The invention provides compositions comprising an RNAi-inducing entity and any of a variety of polymeric delivery agents, including modified polymers, in addition to those described above. The invention further provides methods of inhibiting expression of a transcript in a cell and methods of treating or preventing a disease or condition associated with, characterized by, or featuring excessive or inappropriate expression of particular transcripts or inappropriate or excessive expression or functional activity of a polypeptide encoded by the transcript.

Suitable delivery agents include various agents that have been shown to enhance delivery of DNA to cells. These include modified versions of cationic polymers such as those mentioned above, e.g., poly(L-histidine)-graft-poly(L-lysine) polymers (Benns 2000), polyhistidine-PEG (Putnam 2003), folate-PEG-graft-polyethyleneimine (Benns 2002), polyethylenimine-dextran sulfate (Tiyaboonchai 2003), etc. The polymers may be branched or linear and may be grafted or ungrafted. According to the invention the polymers form complexes with the RNAi-inducing entity, which are then administered to a subject. The complexes may be referred to as nanoparticles or nanocomposites. Any of the polymers may be modified to incorporate PEG or other hydrophilic polymers, which is useful to reduce complement activation and binding of other plasma proteins. Cationic polymers may be multiply modified. For example, a cationic polymer may be modified to incorporate a moiety that reduces the negative charge of the polymer (e.g., imidazole) and may be further modified with a second moiety such as PEG.

In addition, a variety of polymers and polymer matrices distinct from the cationic polymers described above may also be used. Such polymers include a number of non-cationic polymers, i.e., polymers not having positive charge at physiological pH. Such polymers may have certain advantages, e.g., reduced cytotoxicity and, in some cases, FDA approval. A number of suitable polymers have been shown to enhance drug and gene delivery in other contexts. Such polymers include, for example, poly(lactide) (PLA), poly(glycolide) (PLG), and poly(DL-lactide-co-glycolide) (PLGA) (Panyam 2002), which can be formulated into nanoparticles for delivery of inventive RNAi-inducing entities. Copolymers and combinations of the foregoing may also be used. In certain embodiments of the invention a cationic polymer is used to condense the siRNA, shRNA, or vector, and the condensed complex is protected by PLGA or another non-cationic polymer. Other polymers that may be used include noncondensing polymers such as polyvinyl alcohol, or poly(N-ethyl-4-vinylpyridium bromide, which may be complexed with Pluronic 85. Other polymers of use in the invention include combinations between cationic and non-cationic polymers. For example, poly(lactic-co-glycolic acid) (PLGA)-grafted poly(L-lysine) (Jeong 2002) and other combinations including PLA, PLG, or PLGA and any of the cationic polymers or modified cationic polymers such as those discussed above, may be used.

D. Delivery Agents Incorporating Delivery-Enhancing Moieties

The invention encompasses modification of any of the delivery agents to incorporate a moiety that enhances delivery of the agent to cells and/or enhances the selective delivery of the agent to cells in which it is desired to inhibit a target transcript. Any of a variety of moieties may be used including, but not limited to, (i) antibodies or antibody fragments that specifically bind to a molecule expressed by a cell in which inhibition is desired, (e.g., a respiratory epithelial cell); (ii) ligands that specifically bind to a molecule expressed by a cell in which inhibition is desired. Preferably the molecule is expressed on the surface of the cell. Monoclonal antibodies are generally preferred.

In the case of respiratory epithelial cells, suitable moieties include antibodies that specifically bind to receptors such as the p2Y2 purinoceptor, bradykinin receptor, urokinase plasminogen activator R, or serpin enzyme complex may be conjugated to various of the delivery agents mentioned above to increase delivery to and selectivity for, respiratory epithelial cells. Similarly, ligands for these various molecules may be conjugated to the delivery agents to increase delivery to and selectivity for respiratory epithelial cells. See, e.g., (Ferrari 2002). One of ordinary skill in the art will be able to identify appropriate molecules expressed by a cell of interest since many such cell-type specific molecules are described in the scientific literature. Suitable molecules include receptors, e.g., for hormones, growth factors, etc. The cognate hormone or growth factor may be a suitable ligand in such cases. As an example of the general approach, the asialoglycoprotein receptor, which is expressed on liver cells (hepatocytes), is a suitable molecule. Moieties such as galactose that bind to this receptor are appropriate to enhance delivery to hepatocytes. CD4 is expressed on the surface of certain classes of T cells. Thus antibodies or ligands for CD4 are appropriate moieties to enhance delivery of inventive compositions to these cells.

In certain preferred embodiments of the invention binding of the antibody or ligand induces internalization of the bound complex. In certain embodiments of the invention the delivery enhancing agent (e.g., antibody, antibody fragment, or ligand), is conjugated to an RNAi-inducing vector (e.g., a DNA vector) to increase delivery or enhance selectivity. Methods for conjugating antibodies or ligands to nucleic acids or to the various delivery agents described herein are well known in the art. See e.g., “Cross-Linking”, Pierce Chemical Technical Library, available at the Web site having URL www.piercenet.com and originally published in the 1994-95 Pierce Catalog and references cited therein and Wong S S, Chemistry of Protein Conjugation and Crosslinking, CRC Press Publishers, Boca Raton, 1991.

E. Surfactants Suitable for Introduction into the Lung

Natural, endogenous surfactant is a compound composed of phospholipids, neutral lipids, and proteins (Surfactant proteins A, B, C, and D) that forms a layer between the surfaces of alveoli in the lung and the alveolar gas and reduces alveolar collapse by decreasing surface tension within the alveoli (77-84). Surfactant molecules spread within the liquid film that bathes the entire cellular covering of the alveolar walls, where they produce an essentially mono-molecular, all pervasive layer thereon. Surfactant deficiency in premature infants frequently results in respiratory distress syndrome (RDS). Accordingly, a variety of surfactant preparations have been developed for the treatment and/or prevention of this condition. Surfactant can be extracted from animal lung lavage and from human amniotic fluid or produced from synthetic materials (see, e.g., U.S. Pat. Nos. 4,338,301; 4,397,839; 4,312,860; 4,826,821; 5,110,806). Various formulations of surfactant are commercially available, including Infasurf® (manufactured by ONY, Inc., Amherst, N.Y.); Survanta® (Ross Labs, Abbott Park, Ill.), and Exosurf Neonatal® (GlaxoSmithKline, Research Triangle Park, N.C.).

As used herein, the phrase “surfactant suitable for introduction into the lung” includes the particular formulations used in the commercially available surfactant products and the inventive compositions described and claimed in the afore-mentioned patent applications and equivalents thereof. In certain embodiments of the invention the phrase includes preparations comprising 10-20% protein and 80-90% lipid both based on the whole surfactant, which lipid consists of about 10% neutral lipid (e.g., triglyceride, cholesterol) and of about 90% phospholipid both based on the same, while the phosphatidylcholine content based on the total phospholipid is 86%, where both “%” and “part” are on the dried matter basis (see U.S. Pat. Nos. 4,388,301 and 4,397,839).

In certain embodiments of the invention the phrase “surfactant suitable for introduction into the lung” includes synthetic compositions, which may be entirely or substantially free of protein, e.g., compositions comprising or consisting essentially of dipalmitoyl phosphatidylcholine and fatty alcohols, wherein the dipalmitoyl phosphatidylcholine (DPPC) constitutes the major component of the surfactant composition while the fatty alcohol comprises a minor component thereof, optionally including a non-toxic nonionic surface active agent such as tyloxapol (see U.S. Pat. Nos. 4,312,860; 4,826,821; and 5,110,806). One of ordinary skill in the art will be able to determine, by reference to the tests described in the afore-mentioned patents and literature, whether any particular surfactant composition is suitable for introduction into the lung. While not wishing to be bound by any theory, it is possible that the ability of surfactant to spread and cover the alveoli facilitates and the composition of surfactant itself, faciitate the uptake of siRNA and/or nucleic acids or viral vectors by cells within the lung.

Infasurf is a sterile, non-pyrogenic lung surfactant intended for intratracheal instillation only. It is an extract of natural surfactant from calf lungs that includes phospholipids, neutral lipids, and hydrophobic surfactant-associated proteins B and C. Infasurf is approved by the U.S. Food and Drug Administration for the treatment of respiratory distress syndrome and is thus a safe and tolerated vehicle for administration into the respiratory tract and lung. Survanta is also an extract derived from bovine lung, while Exosurf Neonatal is a protein-free synthetic lung surfactant containing dipalmitoylphosphatidylcholine, cetyl alcohol, and tyloxapol. Both of these surfactant formulations have also been approved by the U.S. F.D.A. for treatment of respiratory distress syndrome.

As described in Example 6, the inventors have shown that DNA vectors that provide templates for synthesis of shRNAs that serve as precursors to siRNAs targeted to influenza RNAs can inhibit influenza virus production when mixed with Infasurf and administered to mice by intranasal instillation. In addition, as described in Example 5, the inventors showed that infection with lentiviruses expressing the same shRNAs inhibits influenza virus production in cells in tissue culture. These results demonstrate that shRNAs targeted to target transcripts can be delivered to cells and processed into siRNAs that effectively inhibit expression of the target transcript. The results also demonstrate that surfactant materials such as Infasurf, e.g., materials having a composition and/or properties similar to those of natural lung surfactant, are appropriate vehicles for delivery of RNAi-inducing entitities.

The invention therefore provides a composition comprising (i) an RNAi-inducing entity, wherein the RNAi-inducing entity is targeted to an target transcript; and (ii) a surfactant material suitable for introduction into the lung. These inventive compositions may be introduced into the respiratory system in any of a variety of ways including instillation, by inhalation, by aerosol spray, etc. The compositions need not be introduced directly into the lung but may be introduced into any portion of the respiratory system, including the nasal passages, trachea, bronchi, bronchioles, alveoli, etc. Additional components may be included to facilitate such administration. The compositions may be provided together with a device such as a metered dose inhaler or other device typically used for the administration of drugs to the respiratory passages or alveoli. The invention therefore provides a method of inhibiting expression of a target transcript comprising the step of introducing a composition comprising an RNAi-inducing entity, wherein the RNAi-inducing entity is targeted to the target transcript, and a surfactant suitable for introduction into the lung into the respiratory system of the subject, thereby achieving inhibition of expression of the target transcript in the lung.

The invention is not limited to delivery of RNAi-inducing entities to the lung but also encompasses delivery of these molecules to sites elsewhere in the body. As is well known in the art, the lung is an appropriate site for the delivery of a wide variety of compounds to cells, tissues, and organs elsewhere in the body (Agu 2001; Courrier 2002). The large surface area, good vascularization, large capacity for solute exchange, and extreme thinness of the alveolar epithelium can facilitate systemic delivery of molecules. The invention therefore provides a method of inhibiting expression of a target transcript in a mammalian subject comprising the step of introducing a composition comprising an RNAi-inducing entity targeted to the target transcript and a surfactant suitable for introduction into the lung into the respiratory system of the subject, thereby achieving inhibition of expression of the target transcript in at least one body tissue or organ other than the lung.

F. Additional Agents for Delivery of RNAi-inducing Entities to the Lung

The invention encompasses the use of a variety of additional agents and methods to enhance delivery of RNAi-inducing entities to pulmonary epithelial cells. Methods include CaPO₄ precipitation of vectors prior to delivery or administration together with EGTA to cause calcium chelation. Administration with detergents and thixotrophic solutions may also be used. Perfluorochemical liquids may also be used as delivery vehicles. See (Weiss 2002) for further discussion of these methods and their applicability in gene transfer. In addition, the invention encompasses the use of protein/polyethylenimine complexes incorporating an RNAi-inducing entity for delivery to the lung. Such complexes comprise polyethylenimine in combination with albumin (or other soluble proteins). Similar complexes containing plasmids for gene transfer have been shown to result in delivery to lung tissues after intravascular administration (Orson 2002). Protein/PEI complexes comprising an RNAi-inducing entity may also be used to enhance delivery to cells not within the lung.

G. Lipids

As described in Example 8, the inventors have shown that administration of siRNA targeted to an influenza virus transcript by injection into intact chicken embryos in the presence of the lipid agent known as Oligofectamine™ effectively inhibits influenza virus production while administration of the same siRNA in the absence of Oligofectamine did not result in effective inhibition. These results demonstrate the utility of lipid delivery agents for enhancing the efficacy RNAi in intact organisms. The invention therefore provides a method of treating a subject comprising steps of: (a) providing a subject having disease or condition, or a symptom thereof, that is associated with, characterized by, or features inappropriate or excessive expression of the transcript or inappropriate or excessive functional activity of a polypeptide encoded by the transcript; and (b) administering to the subject a composition comprising (i) an RNAi-inducing entity, wherein the RNAi-inducing entity is targeted to the transcript and (ii) a lipid.

H. Target Transcripts

In general, the target transcript can be any RNA molecule. In preferred embodiments of the invention the target transcript is mRNA. The transcript can be an endogenous cellular transcript or can be an agent-specific transcript that is involved in, for example, replication, pathogenicity, or infection by an infectious agent such as a virus.

Agent-specific transcripts that may be targeted in accordance with the invention include the genome of an infectious agent, where the agent has a genome that comprises RNA and/or any other transcript produced during the life cycle of the agent. Preferred targets include transcripts that are specific for the infectious agent and are not found in the host cell. For example, preferred targets may include transcripts that encode agent-specific polymerases, transcription factors, etc. Such molecules are well known in the art, and the skilled practitioner will be able to select appropriate targets based on knowledge of the life cycle of the agent. In this regard useful information may be found in, e.g., Fields' Virology, 4^{th} ed., Knipe, D. et al. (eds.), Philadelphia, Lippincott Williams & Wilkins, 2001.

In some embodiments of the invention a preferred transcript is one that is particularly associated with the virulence of the infectious agent, e.g., an expression product of a virulence gene. Various methods of identifying virulence genes are known in the art, and a number of such genes have been identified. The availability of genomic sequences for large numbers of pathogenic and nonpathogenic viruses, bacteria, etc., facilitates the identification of virulence genes. Similarly, methods for determining and comparing gene and protein expression profiles for pathogenic and non-pathogenic strains and/or for a single strain at different stages in its life cycle agents enable identification of genes whose expression is associated with virulence. Such methods include, for example, subtractive hybridization. Genes that encode proteins that are toxic to host cells would be considered virulence genes and may be preferred targets for RNAi. Transcripts associated with agent resistance to conventional therapies may also be preferred targets.

As is well known in the art, certain host cell transcripts play an important role in the life cycle of infectious agents, and such transcripts are preferred targets according to certain embodiments of the invention. These transcripts include, among others, host cell transcripts that act as or encode (1) receptors or other molecules that are necessary for or facilitate entry and/or intracellular transport of the infectious agent or a portion of the infectious agent such as the genome; (2) cellular molecules that participate in the life cycle of the infectious agent, e.g., enzymes necessary for replication of the infectious agent's genome, enzymes necessary for integration of a retroviral genome into the host cell genome, cell signalling molecules that enhance pathogen entry and/or gene delivery, cellular molecules that are necessary for or facilitate processing of a viral component, viral assembly, and/or viral transport or exit from the cell; (3) molecules whose expression is induced by the agent and that contribute to or suppress a host response such as an inflammatory or immune response, where the effect of enhancing or suppressing the response is deleterious to the host; and (4) proteins that play a role in the synthesis or processing of any of the foregoing transcripts. See, e.g., Greber, U., et al., “Signalling in viral entry”, Cell Mol Life Sci 2002 April;59(4):608-26), Fuller A and Perez-Romero P, “Mechanisms of DNA virus infection: entry and early events”, Front Biosci 2002 Feb. 1;7:d390-406; Fields' Virology, 4^{th} ed., Knipe, D. et al. (eds.), Philadelphia, Lippincott Williams & Wilkins, 2001, and Bacterial Pathogenesis, Williams, et al. (eds.) San Diego, Academic Press, 1998, for representative examples. Although host transcripts (generally corresponding to host cell genes) necessary or important for effective infection, replication, survival, maturation, pathogenicity, etc., of various infectious agents are known in the art and can be identified by reviewing the relevant scientific literature, additional such transcripts are likely to be identified in the future using any of a number of techniques. The importance of a host transcript in the life cycle of an infectious agent may be determined by comparing the ability of the infectious agent to replicate or infect a host cell in the presence or absence of the host cell transcript. For example, cells lacking an appropriate receptor for an infectious agent would generally be resistant to infection with that agent.

Additional preferred targets include transcripts transcribed from any endogenous gene wherein a disease or condition, or a symptom thereof, is associated with, characterized by, or features inappropriate or excessive expression of the transcript or inappropriate or excessive functional activity of a polypeptide encoded by the transcript. As is well known in the art, cancer is frequently associated with inappropriate or excessive expression of certain genes known as oncogenes and/or expression of mutant forms of these genes. Transcripts transcribed using these genes as a template are appropriate targets for siRNAs and shRNAs useful in the treatment of cancer. For example, amplification and overexpression of the Her2/neu oncogene is believed to play an important role in the pathogenesis of certain forms of breast cancer, and antibodies that bind to the Her1/neu protein are approved therapy for such conditions (Ross 2003). Transcripts that encode Her2/neu are therefore appropriate targets for RNAi-based therapy. The high degree of specificity exhibited by RNAi indicates that siRNA or shRNA targeted to mutant transcripts (e.g., transcripts transcribed from a mutant allele) will be able to specifically reduce expression of the mutant transcript while allowing continued expression of the normal transcript. One of ordinary skill in the art will readily be able to identify additional oncogenes whose excessive or inappropriate expression is associated with cancer. See, e.g., Coleman, W. G., and Tsongalis, G. J. (eds.), The Molecular Basis of Human Cancer, Humana Press, 2001; Mendelsohn, J. (ed.), Howley, P., Israel, M., and Liotta, L., Molecular Basis of Cancer, W.B. Saunders, 2001.

As another example, high blood pressure may be associated with excessive functional activity of angiotensin receptors, and accordingly angiotensin receptor antagonists or inhibitors are effective therapies for certain subjects suffering from high blood pressure. In accordance with the invention, transcripts transcribed using the gene that encodes the angiotensin receptor as a template is an appropriate target for siRNA useful in the treatment of hypertension.

In general, where a disease or condition or a symptom thereof is associated with excessive or inappropriate activity of a receptor (possibly due to inappropriate or excessive levels of the ligand), appropriate targets transcripts include transcripts that encode the receptor, transcripts that encode the ligand, or transcripts that encode proteins involved in synthesis of the receptor or ligand. These examples are intended to be representative only. One of ordinary skill in the art will readily be able to determine or ascertain using routine experimentation whether a particular transcript is an appropriate target for treatment or prevention of a given disease, condition, or symptom thereof.

IV. Testing the Efficacy of Compositions Comprising an RNAi-inducing Entity and a Delivery Agent

In various embodiments of the invention compositions comprising (i) an RNAi-inducing entity, wherein the RNAi-inducing entity is targeted to a transcript and (ii) a delivery agent are tested by administering the composition to cells in tissue culture. For purposes of description, the following discussion will refer to siRNAs, but similar considerations apply to testing the efficacy of other RNAi-inducing entities such as shRNAs or RNAi-inducing vectors. The ability of a candidate siRNA to reduce the level of the target transcript may be assessed by measuring the amount of the target transcript using, for example, Northern blots, nuclease protection assays, reverse transcription (RT)-PCR, real-time RT-PCR, microarray analysis, etc. The ability of a candidate siRNA to inhibit expression of a polypeptide encoded by the target transcript (either at the transcriptional or post-transcriptional level) may be measured using a variety of approaches, e.g., antibody-based approaches including, but not limited to, Western blots, immunoassays, flow cytometry, protein microarrays, etc. In general, any method of measuring the amount of either the target transcript or a polypeptide encoded by the target transcript may be used. In general, certain preferred inhibitors reduce the target transcript level at least about 2 fold, preferably at least about 4 fold, more preferably at least about 8 fold, at least about 16 fold, at least about 64 fold or to an even greater degree relative to the level that would be present in the absence of the inhibitor (e.g., in a comparable control cell lacking the inhibitor).

A variety of additional methods of testing the efficacy of inventive compositions ay be employed. For example, inventive compositions may be tested to assess their effect in vitro on cellular responses such as activation of gene transcription, cell division, apoptosis, release of cytokines or other molecules, degranulation, etc., in response to various stimuli. In general, for any of the above tests, cells to which inventive compositions have been delivered (test cells) may be compared with similar or comparable cells that have not received the inventive composition (control cells).

Inventive compositions can be administered to subjects, e.g., rodents, non-human primates, or humans, and cells can be harvested from the subject. The ability of an inventive composition to inhibit expression of the target trancript and/or its encoded protein is measured as above. The efficacy of a composition for the treatment or prevention of diseases or conditions associated with excessive or inappropriate expression of a transcript or inappropriate or excessive expression or activity of a polypeptide encoded by the transcript may be tested by administering the composition to a subject or group of subjects at risk of or suffering from the disease or condition and assessing the ability of the composition to alleviate or prevent one or more symptoms of the disease, e.g., by comparing the severity or incidence of the symptom in subjects that have received the composition with the severity or incidence in subjects that have not received the composition. Where the disease or condition is caused by an infectious agent, e.g., a virus, the inventive composition may be administered either before or after infection with the virus (or both before and after), and the ability of the composition to inhibit or reduce replication or production of the virus or to alleviate one or more symptoms of viral infection may be assessed, as described in the examples for influenza virus.

V. Applications

As described herein, various embodiments of the present invention provide compositions and methods that may be used to inhibit expression of any target transcript within an intact mammalian or avian organism. The invention provides a method of inhibiting expression of a target transcript in a mammalian subject comprising the step of administering to the subject a composition comprising: (i) an RNAi-inducing entity, wherein the RNAi-inducing entity is targeted to the target transcript; and (ii) a delivery agent selected from the group consisting of: cationic polymers, modified cationic polymers, peptide molecular transporters, surfactants suitable for introduction into the lung, lipids, liposomes, non-cationic polymers, modified non-cationic polymers, bupivacaine, and chloroquine.

Inhibiting target transcript expression is useful for a variety of purposes. For example, inhibiting expression of a target transcript in an intact organism sheds light on the normal role of the transcript in the physiology of the organism in a way that is impossible to achieve in tissue culture. Animals in which inhibition of a target transcript is achieved are useful models for the identification of therapeutic agents that may compensate for loss of the transcript or its encoded polypeptide, which may occur in certain diseases. In addition, such animals are useful to determine whether a particular therapeutic agent may function at least in part by inhibiting or activating expression of the transcript or inhibiting or activating the activity of a polypeptide encoded by the transcript.

The compositions of the present invention may be used to prevent or treat any disease or condition associated with overexpression or inappropriate expression of a transcript or inappropriate or excessive expression or activity of a polypeptide encoded by the transcript. The invention therefore provides a method of treating or preventing a disease or condition associated with overexpression or inappropriate expression of a transcript or inappropriate or excessive expression or activity of a polypeptide encoded by the transcript, the method comprising steps of: (i) providing a subject at risk of or suffering from a disease or condition associated with overexpression or inappropriate expression of a transcript or inappropriate or excessive expression or activity of a polypeptide encoded by the transcript; and (ii) administering to the subject a composition comprising an RNAi-inducing entity targeted to the trancript and a delivery agent selected from the group consisting of a delivery agent selected from the group consisting of: cationic polymers, peptide molecular transporters (including arginine or histidine-rich peptides), lipids (including cationic lipids, neutral lipids, and combinations thereof), liposomes, lipopolyplexes, non-cationic polymers, modified non-cationic polymers, chloroquine, and surfactants suitable for introduction into the lung. In various embodiments of the invention the RNAi-inducing agent may be an siRNA, shRNA, or RNAi-inducing vector.

The invention further provides a method of treating or preventing a disease or condition associated with overexpression or inappropriate expression of a transcript or inappropriate or excessive expression or activity of a polypeptide encoded by the transcript, the method comprising steps of: (i) providing a subject at risk of or suffering from a disease or condition associated with overexpression or inappropriate expression of a transcript or inappropriate or excessive expression or activity of a polypeptide encoded by the transcript; and (ii) administering a composition comprising an RNAi-inducing entity, wherein the RNAi-inducing entity is targeted to the trancript, and a delivery agent selected from the group consisting of cationic polymers, modified cationic polymers, peptide molecular transporters (including arginine or histidine-rich peptides), lipids (including cationic lipids, neutral lipids, and combinations thereof), liposomes, lipopolyplexes, non-cationic polymers, modified non-cationic polymers, chloroquine, and surfactants suitable for introduction into the lung. In various embodiments of the invention the RNAi-inducing agent may be an siRNA, shRNA, or RNAi-inducing vector.

Inventive compositions may comprise a single RNAi-inducing entity, targeted to a single site in a single target RNA transcript, or may comprise a plurality of different RNAi-inducing entities (e.g., different siRNAs, shRNA, or RNA-inducing vectors), targeted to one or more sites in one or more target RNA transcripts. In some embodiments of the invention, it will be desirable to utilize compositions containing collections of different RNAi-inducing entities targeted to different transcripts. For example, it may be desirable to use a variety of RNAi-inducing entities directed against transcripts expressed in different cell types. Alternately, it may be desirable to inhibit a number of different transcripts in a single cell type. Either of these strategies may provide a therapeutic benefit while allowing a reduced level of inhibition of any single transcript relative to the degree of inhibition that would be needed to achieve an equivalent therapeutic effect if only a single transcript were inhibited.

According to certain embodiments of the invention, inventive compositions may contain more than one RNAi-inducing entites targeted to a single transcript. To give but one example, it may be desirable to include at least one siRNA or shRNA targeted to coding regions of a target transcript and at least one siRNA or shRNA targeted to the 3′ UTR. This strategy may provide extra assurance that products encoded by the relevant transcript will not be generated because at least one siRNA or shRNA in the composition may target the transcript for degradation while at least one other inhibits the translation of any transcripts that avoid degradation. Such strategies are not limited to use for therapeutic purposes but may be used in general for inhibition of target genes.

As described above, the invention encompasses “therapeutic cocktails”, including, but not limited to, approaches in which multiple RNAi-inducing species are administered and approaches in which a single vector directs synthesis of siRNAs or shRNAs that inhibit multiple targets or of RNAs that may be processed to yield a plurality of siRNAs or shRNAs.

It may be desirable to combine the administration of an RNAi-inducing entity with one or more other therapeutic agents in order to inhibit, reduce, or prevent one or more symptoms or characteristics of the disease or condition. Appropriate agents are described in, Goodman and Gilman's Pharmacological Basis of Therapeutics. In different embodiments of the invention the terms “combined with” or “in combination with” may mean either that the RNAi-inducing entity present in the same mixture as the other agent(s) or that the treatment regimen for an individual includes both one or more RNAi-inducing entities and the other agent(s), not necessarily delivered in the same mixture or at the same time. Preferably the agent is approved by the U.S. Food and Drug Administration for the treatment of a condition associated with inappropriate or excessive expression of a target transcript or inappropriate or excessive expression or activity of a polypeptide encoded by the target transcript.

Gene therapy protocols may involve administering an effective amount of a gene therapy vector capable of directing expression of an inhibitory siRNA or shRNA to a subject. Another approach that may be used alternatively or in combination with the foregoing is to isolate a population of cells, e.g., stem cells or immune system cells from a subject, optionally expand the cells in tissue culture, and administer a gene therapy vector capable of directing expression of an inhibitory siRNA or shRNA to the cells in vitro. The cells may then be returned to the subject. Optionally, cells expressing the siRNA or shRNA can be selected in vitro prior to introducing them into the subject. In some embodiments of the invention a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

The present invention includes the use of inventive compositions comprising RNAi-inducing entities for the treatment of nonhuman species including, but not limited to, dogs, cats, bovines, ovines, swine, horses, and birds.

VI. Pharmaceutical Formulations

Inventive compositions as described above may be administered to a subject or may first be formulated for delivery by any available route including, but not limited to parenteral (e.g., intravenous), intradermal, subcutaneous, oral, nasal, bronchial, opthalmic, transdermal (topical), transmucosal, rectal, and vaginal routes. Certain preferred routes of delivery are discussed above. Inventive pharmaceutical compositions typically include an RNAi-inducing entity, a delivery agent (i.e., a cationic polymer, peptide molecular transporter, surfactant, etc., as described above) in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compositions comprising an RNAi-inducing entity and a delivery agent are preferably delivered in the form of an aerosol spray from a pressured container or dispenser which contains a suitable propellant, e.g., a gas such as carbon dioxide, or a nebulizer. The present invention particularly contemplates delivery of the compositions using a nasal spray, inhaler, or other direct delivery to the upper and/or lower airway. Intranasal administration of DNA vaccines directed against influenza viruses has been shown to induce CD8 T cell responses, indicating that at least some cells in the respiratory tract can take up DNA when delivered by this route, and the delivery agents of the invention will enhance cellular uptake. According to certain embodiments of the invention the compositions comprising an RNAi-inducing entity and a delivery agent are formulated as large porous particles for aerosol administration as described in more detail in Example 3.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds and delivery agents are formulated into ointments, salves, gels, or creams as generally known in the art.

The compositions can also be prepared in the form of suppositories (e.g., with conventional suppository bases such as cocoa butter and other glycerides) or retention enemas for rectal delivery.

In one embodiment, the compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, appropriate care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any composition used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an RNAi-inducing entity as described herein, can include a single treatment or, in many cases, can include a series of treatments.

Exemplary doses include milligram or microgram amounts of the inventive siRNA per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses may depend upon the potency of the RNAi-inducing entity and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

As mentioned above, the present invention includes the use of inventive compositions for treatment of nonhuman animals. Accordingly, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001.

As described above, nucleic acid molecules that serve as templates for transcription of siRNA or shRNA can be inserted into vectors which can be used as gene therapy vectors. In general, gene therapy vectors can be delivered to a subject by, for example, intravenous injection, local administration, or by stereotactic injection (see e.g., Chen et al. (1994) Proc. Natl. Acad. Sci. USA 91:3054-3057). In certain embodiments of the invention compositions comprising gene therapy vectors and a delivery agent may be delivered orally or inhalationally and may be encapsulated or otherwise manipulated to protect them from degradation, etc. The pharmaceutical compositions comprising a gene therapy vector can include an acceptable diluent, or can comprise a slow release matrix in which the gene delivery vehicle is imbedded. Alternatively, where the complete gene delivery vector can be produced intact from recombinant cells, e.g., retroviral or lentiviral vectors, the pharmaceutical preparation can include one or more cells which produce the gene delivery system.

Inventive pharmaceutical compositions can be included in a container, pack, or dispenser together with instructions for administration.

### Exemplification

### EXAMPLE 1

Evaluation of non-viral delivery agents that facilitate cellular uptake of siRNA. This example describes testing a variety of non-viral delivery agents for their ability to enhance delivery of siRNA to cells in tissue culture and in intact organisms. Subsequent examples provide data showing positive results in tissue culture and in vivo with a number of the polymers that were tested as described below and in the examples themselves.

siRNA Selection and Design

In general, an siRNA may be selected according to the criterial described above. For example, according to one approach, a target region within the RNA whose expression is to be inhibited is selected. For purposes of description, it is assumed that the target region is 19 nt in length and that the region is selected by reference to the sequence of an mRNA or cDNA, where the cDNA has the same sequence as the mRNA, which sequence is referred to as the sense sequence (except that the cDNA contains T rather than U). The sense strand of the siRNA has the same sequence as the 19 nucleotide region, and the antisense strand of the siRNA has a sequence perfectly complementary to the sense strand. Each strand further includes a two nt 3′ overhang consisting of dTdT. Hybridization of the sense and antisense strands results in an siRNA having a 19 base pair core duplex region, with each strand having a 2 nucleotide 3′ OH overhang.

Cationic polymers. As mentioned above, the ability of cationic polymers to promote intracellular uptake of DNA is believed to result partly from their ability to bind to DNA and condense large plasmid DNA molecules into smaller DNA/polymer complexes for more efficient endocytosis. siRNA duplexes are short (e.g., only approximately 19-21 nucleotides in length), suggesting that they probably cannot be condensed much further. siRNA precursors, such as shRNAs, are also relatively short. However, the ability of cationic polymers to bind negatively charged siRNA and interact with the negatively charged cell surface may facilitate intracellular uptake of siRNAs. Thus, known cationic polymers including, but not limited to, PLL, modified PLL (e.g., modified with acyl, succinyl, acetyl, or imidazole groups (32)), polyethyleneimine (PEI) (37), polyvinylpyrrolidone (PVP) (38), and chitosan (39, 40) are promising candidates as delivery agents for siRNA and shRNA.

In addition, novel cationic polymers and oligomers developed in Robert Langer's laboratory at the Massachusetts Institute of Technology are promising candidates as delivery agents. Efficient strategies to synthesize and test large libraries of novel cationic polymers and oligomers from diacrylate and amine monomers for their use in DNA transfection have been developed. These polymers are referred to herein as poly(β-amino ester) (PAE) polymers. In a first study, a library of 140 polymers from 7 diacrylate monomers and 20 amine monomers was synthesized and tested (34). Of the 140 members, 70 were found sufficiently water-soluble (2 mg/ml, 25 mM acetate buffer, pH=5.0). Fifty-six of the 70 water-soluble polymers interacted with DNA as shown by electrophoretic mobility shift. Most importantly, two of the 56 polymers were found to mediate DNA transfection into COS-7 cells. Transfection efficiencies of the novel polymers were 4-8 times higher than PEI and equal or better than Lipofectamine 2000.

Since the initial study, a library of 2,400 cationic polymers has been constructed and screened, and another approximately 40 polymers that promote efficient DNA transfection have been obtained (93). Because structural variations could have a significant impact on DNA binding and transfection efficacies (33), it is preferable to test many polymers for their ability to promote intracellular uptake of siRNA. Furthermore, it is possible that in the transition to an in vivo system, i.e., in mammalian subjects, certain polymers will likely be excluded as a result of studies of their in vivo performance, absorption, distribution, metabolism, and excretion (ADME). Thus testing is intact organisms is important.

Together, at least approximately 50 cationic polymers will be tested in siRNA transfection experiments. Most of them will be PAE and imidazole group-modified PLL as described above. PEI, PVP, and chitosan will be purchased from commercial sources. To screen these polymers rapidly and efficiently, the library of PAE polymers that successfully transfects cells has already been moved into solution into a 96-well plate. Storage of the polymers in this standard 96 well format allows for the straightforward development of a semi-automated screen, using a sterile Labcyte EDR 384S/96S micropipettor robot. The amount of polymer will be titrated (using a predetermined amount of siRNA) to define proper polymer siRNA ratios and the most efficient delivery conditions. Depending on the specific assay, the semi-automated screen will be slightly different as described below.

Characterization of siRNA/polymer complexes. For various cationic polymers to facilitate intracellular uptake of siRNA, they should be able to form complexes with siRNA. This issue will be examined this by electrophoretic mobility shift assay (EMSA) following a similar protocol to that described in (34). Briefly, an siRNA will be mixed with each of the polymers at various ratios, e.g., 1:0.1, 1:0.3, 1:0.9, 1:2.7, 1:8.1, and 1:24.3 (siRNA/polymer, w/w) in 96-well plates using micropipettor robot. The mixtures will be loaded into 4% agarose gel slab capable of assaying up to 500 samples using a multichannel pipettor. Migration patterns of siRNA will be visualized by ethidium bromide staining. If the mobility of an siRNA is reduced in the presence of a polymer, the siRNA forms complexes with that polymer. Based on the ratios of siRNA to polymer, it may be possible to identify the neutralizing ratio. Those polymers that do not bind siRNA will be less preferred and further examination will focus on those polymers that do bind siRNA.

Cytotoxicity of imidazole group-modified PLL, PEI, PVP, chitosan, and some PAE polymers has been measured alone or in complexes with DNA in cell lines. Because cytotoxicity changes depending on bound molecules, the cytotoxicity of various polymers in complexes with siRNA will be measured in MDCK cells. Briefly, an siRNA will be mixed with different amounts of polymers as above, using the sterile Labcyte micropipettor robot. The complexes will be applied to MDCK cells in 96-well plates for 4 hrs. Then, the polymer-containing medium will be replaced with normal growth medium. 24 hrs later, the metabolic activity of the cells will be measured in the 96-well format using the MTT assay (41). A variety of different siRNAs may be tested to avoid effects due to possible inhibition of gene expression due to the siRNA. Those polymers that kill 90% or more cells at the lowest amount used will be less preferred, and the focus of further investigation will be polymers that do not kill more than 90% of the cells at the lowest amount used. In general, polymers that exhibit minimal effect on cell growth or proliferation are preferred.

While in some cases similar studies have been performed using DNA/polymer compositions, it will be important to determine whether similar results (e.g., cytotoxicity, promotion of cellular uptake) are obtained with RNA/polymer compositions.

siRNA uptake by cultured cells. Once siRNA/polymer complexes have been characterized, their ability to promote cellular uptake of siRNA will be tested, starting with cultured cells using two different assay systems. In the first approach, a GFP-specific siRNA (GFP-949) will be tested on GFP-expressing MDCK cells, because a decrease in GFP expression is easily quantified by measuring fluorescent intensity. Briefly, GFP-949/polymer at the same ratios as above will be applied to MDCK cells in 96-well plates. As negative controls, an unrelated siRNA (e.g., NP-1496 siRNA, described below) or no siRNA will be used. As a positive control, GFP-949 will be introduced into cells by electroporation, which has previously been shown to be an effective means of introducing siRNA into cells. 36 hrs later, cells will be lysed in 96-well plates and fluorescent intensity of the lysates measured by a fluorescent plate reader. The capacities of various polymers to promote cellular uptake of siRNA will be indicated by the overall decrease of GFP intensity. Alternatively, cells will be analyzed for GFP expression using a flow cytometer that is equipped to handle samples in the 96-well format. The capacities of various polymers to promote cellular uptake of siRNA will be indicated by percentage of cells with reduced GFP intensity and the extent of decrease in GFP intensity. Results from these assays will also shed light on the optimal siRNA:polymer ratio for most efficient transfection.

As described in co-pending U.S. patent application entitled “Influenza Therapeutic”, filed on even date herewith, siRNA targeted to influenza virus transcripts inhibits production of influenza virus when introduced into cells by electroporation. For example, the inventors showed that an siRNA targeted to the viral NP (nucleoprotein) transcript, referred to as NP-1496, significantly inhibited production of influenza virus when applied to cells in tissue culture prior to infection with influenza virus and when administered to mice either before or after infection with influenza virus. Thus influenza virus infection in cells or intact organisms provide appropriate systems in which to test the efficacy of inventive siRNA/delivery agent compositions.

To further characterize such compositions, their ability to inhibit influenza virus production in MDCK cells will be measured. As described above, NP-1496 siRNA/polymer at various ratios will be applied to MDCK cells in 96-well plates. As a positive control, siRNA will be introduced into MDCK cells by electroporation. As negative controls, GFP-949 with or without polymer, polymer alone, or no treatment will be used. NP-1496 without polymer may also be used for purposes of comparison. Eight hrs later, cells will be infected with PR8 or WSN virus at a predetermined MOI. Culture supernatants will be harvested 60 hrs later and assayed for virus without dilution by hemagglutination in 96-well plates. Supernatants from wells that have low virus titers in the initial assay will be diluted and assayed by hemagglutination. Alternatively, infected cultures at 60 hrs will be assayed for metabolic activity by the MTT assay. Because infected cells eventually lyse, the relative level of metabolic activity should also give an indication of inhibition of virus infection.

If the virus titer or metabolic activity is substantially lower in cultures that are treated with siRNA/polymer than those that are not treated with such compositions, it will be concluded that the polymer promotes siRNA transfection. By comparing the virus titers in cultures in which siRNA is introduced by electroporation, the relative transfection efficiency of siRNAs and siRNA/polymer compositions will be estimated.

A number of the most effective cationic polymers from the initial two screens will be verified in the virus infection assay in 96-well plates by titrating both siRNA and polymers. Based on the results obtained, the capacity of the six polymers at the most effective siRNA:polymer ratios will be further analyzed in MDCK cells in 24-well plates and 6-well plates. A number of the most effective polymers will be selected for further studies in mice as described in Example 2.

Arginine-rich peptides. As an alternative to cationic polymers for efficient promotion of intracellular uptake of siRNA in cultured cells, arginine-rich peptides will be investigated in siRNA transfection experiments. Because ARPs are thought to directly penetrate the plasma membrane by interacting with the negatively charged phospholipids (48), whereas most currently used cationic polymers are thought to promote cellular uptake of DNA by endocytosis, the efficacy of ARPs in promoting intracellular uptake of siRNA will be, investigated. Like cationic polymers, ARPs and polyarginine (PLA) are also positively charged and likely capable of binding siRNA, suggesting that it is probably not necessary to covalently link siRNA to ARPs or PLAs. Therefore, ARPs or PLAs will be treated similarly to other cationic polymers. The ability of the ARP from Tat and different length of PLAs (available from Sigma) to promote cellular uptake of siRNA will be determined as described above.

### EXAMPLE 2

### Testing of siRNAs and siRNA/Delivery Agent Compositions in Mice

Rationale: The ability of identified polymers to promote siRNA delivery in mice will be evaluated, and the efficacies of siRNAs in preventing and treating influenza virus infection in mice will be examined. Demonstration of inhibition of influenza virus infection in mice by compositions comprising an siRNA and a delivery agent will provide evidence that the compositions promote delivery of the siRNAs to cells within the body, where they then act to inhibit expression of target transcripts. Positive results with the influenza virus system strongly suggest that the compositions will effectively inhibit any cellular or viral transcript. Methodology for identifying siRNA-containing compositions that effectively deliver siRNA to cells in the body are described in this Example. For simplicity the Example describes testing of siRNA/polymer compositions. Analogous methods may be used for testing of other inventive compositions such as siRNA/cationic polymer compositions, siRNA/arginine-rich peptide compositions, etc.

Routes of administration. One route by which siRNAs may be delivered to cells within the body is via the lungs. Many different methods have been used to deliver small molecule drugs, proteins, and DNA/polymer complexes into the upper airways and/or lungs of mice, including instillation, aerosol (both liquid and dry-powder) inhalation, intratracheal administration, and intravenous injection. By instillation, mice are usually lightly anesthetized and held vertically upright. Therapeutics (i.e. siRNA/polymer complexes) in a small volume (usually 30-50 μl) are applied slowly to one nostril where the fluid is inhaled (52). The animals are maintained in the upright position for a short period of time to allow instilled fluid to reach the lungs (53). Instillation is effective to deliver therapeutics to both the upper airways and the lungs and can be repeated multiple times on the same mouse.

By aerosol, liquid and dry-powder are usually applied differently. Liquid aerosols are produced by a nebulizer into a sealed plastic cage, where the mice are placed (52). Because aerosols are inhaled as animals breathe, the method may be inefficient and imprecise. Dry-powder aerosols are usually administered by forced ventilation on anesthetized mice. This method can be very effective as long as the aerosol particles are large and porous (see below) (31). For intratracheal administration, a solution containing therapeutics is injected via a tube into the lungs of anesthetized mice (54). Although it is quite efficient for delivery into the lungs, it misses the upper airways. Intravenous injection of a small amount of DNA (˜1 μg) in complexes with protein and polyethyleneimine has been shown to transfect endothelial cells and cells in interstitial tissues of the lung (55). Based on this consideration, siRNA/polymer complexes will first be administered to mice by instillation. Intravenous delivery and aerosol delivery using large porous particles will also be explored.

siRNA uptake by cells in the respiratory tract. A number of the most effective polymers identified as described in Example 1 will be tested for their ability to promote intracellular uptake of siRNA in the respiratory tract in mice. To facilitate investigations, inhibition of GFP expression by GFP-specific siRNA (GFP-949) in GFP-expressing transgenic mice will be used. The advantage of using GFP-specific siRNA initially is that the simplicity and accuracy of the assays may speed up the identification of effective polymers in mice. In addition, the results obtained may shed light on the areas or types of cells that take up siRNA in vivo. The latter information will be useful for modifying delivery agents and methods of administration for optimal delivery of siRNA by the pulmonary route.

Briefly, graded doses of GFP-949/polymer complexes (at the most effective ratio as determined in Example 1) will be administered to GFP transgenic mice by instillation. As controls, mice will be given siRNA alone, or polymers alone, or nothing, or non-specific siRNA/polymer complexes. Tissues from the upper airways and the lung will be harvested 36 to 48 hrs after siRNA administration, embedded in OCT, and frozen. Sections will be visualized under a fluorescence microscope for the GFP intensity, and adjacent sections will be stained with hematoxylin/eosin (H/E). Alternatively, tissues will be fixed in paraformaldehyde and embedded in OCT. Some sections will be stained with H&E and adjacent sections will be stained with HRP-conjugated anti-GFP antibodies. Overlay of histology and GFP images (or anti-GFP staining) may be able to identify the areas or cell types in which GFP expression is inhibited. For increased sensitivity, the tissues may be examined by confocal microscopy to identify areas where GFP intensity is decreased.

Based on findings from DNA transfection by instillation (52, 56), it is expected that siRNA will be most likely taken up by epithelial cells on the luminal surface of the respiratory tract. If a significant decrease in GFP intensity is observed in GFP-949/polymer treated mice compared to control mice, this would indicate that the specific polymer promotes cellular uptake of siRNA in vivo.

In addition to delivery of siRNA to epithelial cells in the respiratory tract, siRNAs may also be delivered to other regions of the body via the lung. The extensive capillary system of the lung offers a large surface area through which molecules may enter the bloodstream and subsequently be transported throughout the body. It is well known that pulmonary administration of a variety of compounds including small molecules, nucleic acids, polypeptides results in distribution to other tissues or organs in the body. Thus the ability of compositions comprising siRNA and a cationic polymer delivered to the respiratoy system to inhibit expression of target transcripts elsewhere in the body will be similarly evaluated.

siRNA inhibition of influenza virus infection in mice. In addition to the above GFP-949 study in GFP transgenic mice, a number of the most effective polymers in promoting siRNA uptake in mice will be examined using siRNA specific for influenza virus, such as NP-1496 or more likely two or three siRNA “cocktails”. For the initial study, siRNA/polymer complexes and influenza virus will be introduced into mice at the same time by mixing siRNA/polymer complexes and virus before instillation. Graded doses of siRNA/polymer complexes and PR8 virus (at a predetermined dose) will be used. As controls, mice will be given siRNA alone, or polymers alone, or nothing, or GFP-949/polymer. 2 or 3 days after infection, nasal lavage will be prepared and lungs will be homogenized to elute virus by freeze and thaw. The virus titer in the lavage and the lungs will be measured by hemagglutination. If the titer turns out to be too low to detect by hemagglutinin assay, virus will be amplified in MDCK cells before hemagglutinin assay. For more accurate determination of virus titer, plaque assays will be performed on selected samples.

If a single dose of siRNA/polymer is not effective in inhibiting influenza infection, multiple administrations of siRNA (at a relatively high dosage) will be investigated to determine whether multiple administrations are more effective. Following the initial siRNA/polymer and virus administration, mice will be given siRNA/polymer every 12 hrs for 2 days (4 doses). The titer of virus in the lung and nasal lavage will be measured on day 3 after the initial infection.

Results from these experiments should show whether siRNAs are effective in inhibiting influenza virus infection in the upper airways and the lungs, and point to the most effective single dose. It is expected that multiple administrations of siRNA/polymer are likely to be more effective than a single administration in treating influenza virus infection. If negative results are obtained using both siRNAs specific for GFP other effective polymers or delivery agents will be explored as well as a different approach for siRNA/polymer delivery as described below.

siRNA/polymer delivery using large porous particles. Another efficient delivery method to the upper airway and the lungs is using large porous particles originally developed by Robert Langer's group. In contrast to instillation, which is liquid-based, the latter method depends on inhalation of large porous particles (dry-powder) carrying therapeutics. In initial studies, it was demonstrated that double-emulsion solvent evaporation of therapeutics and poly(lactic acid-co-glycolic acid) (PLGA) or poly(lactic acid-co-lysine-graft-lysine) (PLAL-Lys) leads to the generation of large porous particles (31). These particles have mass densities less than 0.4 gram/cm³ and mean diameters exceeding 5 μm. They can be efficiently inhaled deep into the lungs because of their low densities. They are also less efficiently cleared by macrophages in the lungs (57). Inhalation of large porous insulin-containing particles by rats results in elevated systemic levels of insulin and suppression of systemic glycose levels for 96 hrs, as compared to 4 hrs by small nonporous particles.

Since their initial study, the Langer group has developed a procedure for producing large porous particles using excipients that are either FDA-approved for inhalation or endogenous to the lungs (or both) (58). In this procedure, water-soluble excipients (i.e. lactose, albumin, etc.) and therapeutics were dissolved in distilled water. The solution was fed to a Niro Atomizer Portable Spray Dryer (Niro, Inc., Colombus, Md.) to produce the dry powders, which have a mean geometric diameters ranged between 3 and 15 μm and tap density between 0.04 and 0.6 g/cm³.

The spray-dry method will be used to produce large porous low-density particles carrying siRNA/polymer described by Langer except that the therapeutics are replaced with siRNA/polymer. The resulting particles will be characterized for porosity, density, and size as described in (31, 58). Those that reach the aforementioned criteria will be administered to anesthetized mice by forced ventilation using a Harvard ventilator. Depending on whether siRNA specific for either GFP or influenza virus is used, different assays will be performed as described above. If GFP expression or the virus titer in mice that are given specific siRNA/polymer in large porous particles is significantly lower than in control mice, aerosol inhalation via large porous particles would appear to be an effective method for siRNA delivery.

Prophylactic and therapeutic application of siRNA/polymer complexes. The efficacy of siRNA/polymer complexes as prophylaxis or therapy for influenza virus infection in mice will be examined. Assuming a single dose of siRNA/polymer complexes is effective, the length of time after their administration over which the siRNAs remain effective in interfering with influenza infection will be assessed. siRNA/polymer complexes will be administered to mice by instillation or large porous aerosols (depending on which one is more effective as determined above). Mice will be infected with influenza virus immediately, or 1, 2, or 3 days later, and virus titer in the nasal lavage and the lung will be measured on 24 or 48 hrs after virus infection. If siRNA is found to be still effective after 3 days, mice will be infected 4, 5, 6, and 7 days after siRNA/polymer administration, and tissues will be harvested for assaying virus titer 24 hrs after the infection. Results from these experiments will likely reveal how long after administration, siRNAs remain effective in interfering with transcript expression in mice and will guide siRNA use in humans in general.

To evaluate therapeutic efficacy of siRNAs, mice will be infected with influenza virus and then given siRNA/polymer complexes at different times after infection. Specifically, mice will be infected intranasally, and then given an effective dose (as determined above) of siRNA/polymer immediately, or 1, 2, or 3 days later. As controls, mice will be given GFP-949 or no siRNA at all immediately after infection. The virus titer in the nasal lavage and the lung will be measured 24 or 48 hrs after siRNA administration.

In addition, mice will be infected with a lethal dose of influenza virus and into five groups (5-8 mice per group). Group 1 will be given an effective dose of siRNA/polymer complexes immediately. Groups 2 to 4 will be given an effective dose of siRNA/polymer complexes on day 1 to 3 after infection, respectively. Groups 5 will be given GFP-specific siRNA immediately after infection and used as controls. Survival of the infected mice will be followed. Results from these experiments will likely reveal how long after infection administration of siRNAs still exerts a therapeutic effect in mice. Since the efficacy of siRNAs targeted to influenza virus transcripts depends on their ability to inhibit expression of the target transcripts, the results obtained from these experiments and the methods described above may be generalized to develop appropriate therapeutic regimens for the inhibition of other target transcripts.

### EXAMPLE 3

### Inhibition of Influenza Virus Infection by siRNAs Transcribed from DNA Vectors

Rationale: Effective siRNA therapy depends on the ability to deliver a sufficient amount of siRNA into appropriate cells in vivo. For example, in the case of influenza virus infection, to prevent the emergence of resistant virus, it may be preferable to use two or three siRNAs together. Simultaneous delivery of two or three siRNAs into the same cells will require an efficient delivery system. As an alternative to the approaches described above, the use of DNA vectors from which siRNA precursors can be transcribed and processed into effective siRNAs will be explored.

We have previously shown that siRNA transcribed from a DNA vector can inhibit CD8α expression to the same extent as synthetic siRNA introduced into the same cells. Specifically, we found that one of the five siRNAs designed to target the CD8α gene, referred to as CD8-61, inhibited CD8 but not CD4 expression in a mouse CD8⁺CD4⁺ T cell line (27). By testing various hairpin derivatives of CD8-61 siRNA, we found that CD8-61F had a similar inhibitory activity as CD8-61 (59). Because of its hairpin structure, CD8-61F was constructed into pSLOOP III, a DNA vector in which CD8-61F was driven by the H1 RNA promoter. The H1 RNA promoter is compact (60) and transcribed by polymerase III (pol III). The Pol III promoter was used because it normally transcribes short RNAs and has been used to generate siRNA-type silencing previously (61). To test the DNA vector, we used HeLa cells that had been transfected with a CD8α expressing vector. Transient transfection of the pSLOOP III-CD8-61F plasmid into CD8α-expressing HeLa cells resulted in reduction of CD8α expression to the same extent as HeLa cells that were transfected with synthetic CD8-61 siRNA. In contrast, transfection of a promoter-less vector did not significantly reduce CD8α expression. These results show that a RNA hairpin can be transcribed from a DNA vector and then processed into siRNA for RNA silencing. A similar approach is used to design DNA vectors that express siRNA precursors specific for the influenza virus, as described herein.

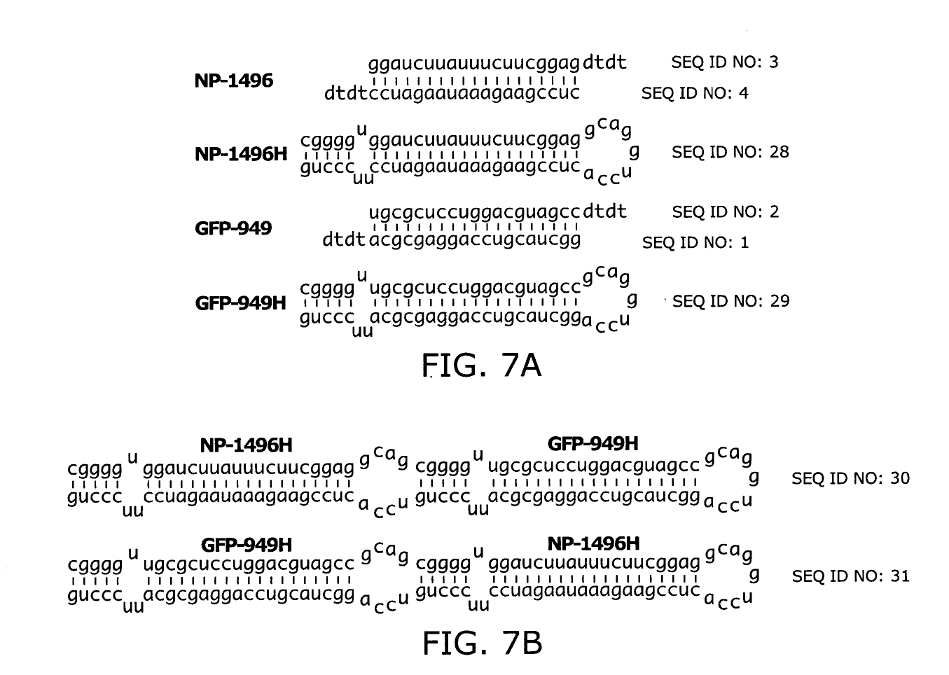

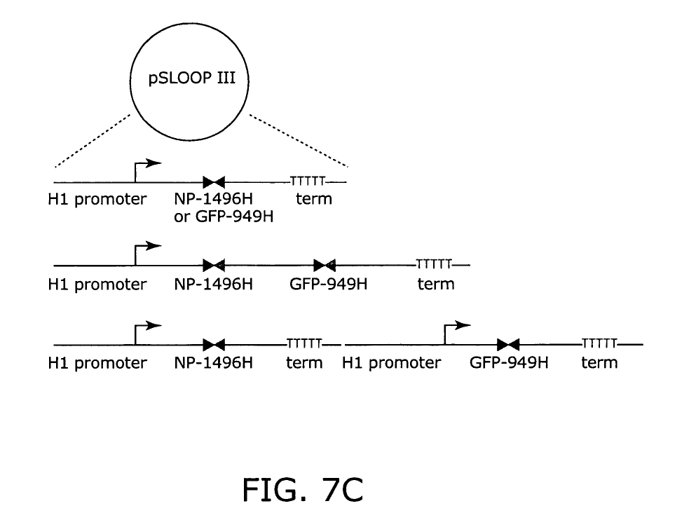

Investigation of siRNA transcribed from DNA templates in cultured cells. To express siRNA precursors from a DNA vector, hairpin derivatives of siRNA (specific for influenza virus) that can be processed into siRNA duplexes will be designed. In addition, vectors from which two or more siRNA precursors can be transcribed will be produced. To speed up these investigations, GFP-949 and NP-1496 siRNAs will be used in MDCK cells that express GFP. Following the CD8-61F design, hairpin derivatives of GFP-949 and NP-1496, referred to as GFP-949H and NP-1496H, respectively will be synthesized (FIG. 7A).

Both GFP-949 and GFP-949H will be electroporated into GFP-expressing MDCK cells. NP-1496 or mock electroporation will be used as negative controls. 24 and 48 hrs later, cells will be assayed for GFP expression by flow cytometry. If the percentage of GFP-positive cells and the intensity of GFP level are significantly reduced in cultures that are given GFP-949H, the hairpin derivative's effectiveness will have been demonstrated. Its efficacy will be indicated by comparing GFP intensity in cells given standard GFP-949.

Similarly, NP-1496 and NP-1496H will be electroporated into MDCK cells. GFP-949 or mock electroporation will be used as negative controls. 8 hrs later after transfection, cells will be infected with PR8 or WSN virus. The virus titers in the culture supernatants will be measured by hemagglutination 60 hrs after the infection. If the virus titer is significantly reduced in cultures given NP-1496H, the hairpin derivative inhibits virus production. It is expected that the hairpin derivatives will be functional based on studies with CD8-61F. If not, different designs of hairpin derivatives similar to those described in (59, 61, 62) will be synthesized and tested.

Designing DNA vectors and testing them in cultured cells. Once GFP-949H and NP-1496H are shown to be functional, the corresponding expression vectors will be constructed. GFP-949H and NP-1496H will be cloned individually behind the H1 promoter in the pSLOOP III vector (FIG. 7C, top). The resulting vectors will be transiently transfected into GFP-expressing MDCK cells by electroporation. Transfected cells will be analyzed for GFP intensity or infected with virus and assayed for virus production. The U6 Pol III promoter, which has also been shown to drive high levels of siRNA precursor expression will be tested this in addition to other promoters to identify a potent one for siRNA precursor transcription.

Once vectors that transcribe a single siRNA precursor are shown to be effective, vectors that can transcribe two siRNA precursors will be constructed. For this purpose, both GFP-949H and NP-1496H will be cloned into pSLOOP III vector in tandem, either GFP-949H at the 5′ and NP-1496H at the 3′, or the other way around (FIG. 7C, middle). In the resulting vectors, the two siRNA precursors will be linked by extra nucleotides present in the hairpin structure (FIG. 21B). Because it is not known whether two siRNAs can be processed from a single transcript, vectors in which both GFP-949H and NP-1496H are transcribed by independent promoters will also be constructed (FIG. 7C, bottom).

Because transfection efficiency in MDCK cells is about 50%, transient transfection may not be ideal for evaluating vectors that encode two siRNA precursors. Therefore, stable transfectants will be established by electroporating GFP-expressing MDCK cells with linearized vectors plus a neo-resistant vector. DNA will be isolated from multiple transfectants to confirm the presence of siRNA expressing vectors by Southern blotting. Positive transfectants will be assayed for GFP expression to determine if GFP-specific siRNA transcribed from the stably integrated vector can inhibit GFP expression. Those transfectants in which GFP expression is inhibited will be infected with PR8 or WSN virus and the virus titer will be measured by hemagglutination. The finding that both GFP expression and virus production are inhibited in a significant fraction of transfectants would establish that two siRNA precursors can be transcribed and processed from a single DNA vector.

Constructing vectors from which a single siRNA precursor will be transcribed should be straightforward because a similar approach has been successfully used in previous studies (59). Since many studies have shown that two genes can be transcribed independently from the same vector using identical promoter and termination sequences, it is likely that two siRNA precursors can be transcribed from the same vector. In the latter approach, siRNA precursors are independently transcribed. The length of the resulting dsRNA precursors is likely less than 50 nucleotides. In contrast, when two siRNA precursors are transcribed in tandem (FIGS. 7B and C), the resulting dsRNA precursor would be longer than 50 nucleotides. The presence of dsRNA longer than 50 nucleotides activates an interferon response in mammalian cells (22, 23). Thus, another advantage of independent transcription of two siRNA precursors from the same vector is that it would avoid an interferon response. Interferon inhibits virus infection and therefore could be useful, but the response also shuts down many metabolic pathways and therefore interferes with cellular function (63).

To determine if an interferon response is induced in MDCK cells transfected with various DNA vectors, the level of total and phosphorylated dsRNA-dependent protein kinase (PKR) will be assayed since phosphorylation of PKR is required for the interferon response (23). Cell lysates prepared from vector- and mock-transfected cells will be fractionated on SDS-PAGE. Proteins will be transferred onto a membrane and the membrane probed with antibodies specific to phosphorylated PKR or total PKR. If the assay is not sufficiently sensitive, immunoprecipitation followed by Western blotting will be performed. If no difference in the level of activated PKR is detected, dsRNA precursors transcribed from the DNA vectors do not activate the interferon response. Preferred DNA vectors for intracellular synthesis of siRNAs do not activate the interferon response, and the invention thus provides such vectors.

Investigation of DNA vectors in mice. Once it is shown that siRNA transcribed from DNA vectors can inhibit influenza virus production in MDCK cells, their efficacies in mice will be investigated. To minimize the integration of introduced plasmid DNA into the cellular genome, supercoiled DNA will be used for transient expression. The other advantage of transient expression is that the level of expression tends to be high, probably because the plasmid copy numbers per cell is high prior to integration. To facilitate DNA transfection in mice, cationic polymers that have been developed for gene therapy, including imidozole group-modified PLL, PEI, PVP, and PAE as described in Example 1, will be used.

Specifically, DNA vectors expressing GFP-949H or NP-1496H alone or both NP-1496H and GFP-949H will be mixed with specific polymers at a predetermined ratio. Graded amounts of the complexes plus PR8 or WSN virus will be introduced into anesthetized GFP transgenic mice by instillation. As controls, mice will be given DNA alone, or polymers alone, or nothing. Two and three days after infection, nasal lavage and lungs will be harvested for assaying for virus titer as described in Example 2. In addition, the upper airways and the lung sections will be examined for reduction in GFP expression.

DNA/polymer complexes will also be administered multiple times, e.g. together with the virus initially and once a day for the following two days. The effect of multiple administrations will be examined on day 3 after the infection. In addition, DNA vectors that encode two or three influenza-specific siRNA precursors will be constructed and their efficacies in inhibiting influenza infection in mice will be tested.

### EXAMPLE 4

### Inhibition of Influenza Virus Production in Mice by siRNAs

This example describes experiments showing that administration of siRNAs targeted to influenza virus NP or PA transcripts inhibit production of influenza virus in mice when administered either prior to or following infection with influenza virus. The inhibition is dose-dependent and shows additive effects when two siRNAs targeted to transcripts expressed from two different influenza virus genes were administered together.

Materials and Methods

SiRNA preparation. All siRNAs were synthesized by Dharmacon Research (Lafayette, Colo.) using 2'ACE protection chemistry. The siRNA strands were deprotected according to the manufacturer's instructions, mixed in equimolar ratios and annealed by heating to 95° C. and slowly reducing the temperature by 1° C. every 30 s until 35° C. and 1° C. every min until 5° C. The sequences of sense and antisense siRNA strands used in this and following examples (including an appended dTdT overhang), reading in the 5′ to 3′ direction were as follows:

| | | |
|---|---|---|
| GFP-949 sense | GGCUACGUCCAGGAGCGCAdTdT | (SEQ ID NO: 1) |
| GFP-949 antisense | UGCGCUCCUGGACGUAGCCdTdT | (SEQ ID NO: 2) |
| NP-1496 sense | GGAUCUUAUUUCUUCGGAGdTdT | (SEQ ID NO: 3) |
| NP-1496 antisense | CUCCGAAGAAAUAAGAUCCdTdT | (SEQ ID NO: 4) |
| PA-2087/2107 (G) sense | GCAAUUGAGGAGUGCCUGAdTdT | (SEQ ID NO: 5) |
| PA-2087/2107 (G) antisense | UCAGGCACUCCUCAAUUGCdTdT | (SEQ ID NO: 6) |
| PB1-2257/2277 sense | GAUCUGUUCCACCAUUGAAdTdT | (SEQ ID NO: 7) |
| PB1-2257/2277 antisense | UUCAAUGGUGGAACAGAUCdTdT | (SEQ ID NO: 8) |
| NP-231/251 sense | UAGAGAGAAUGGUGCUCUCdTdT | (SEQ ID NO: 9) |
| NP-231/251 antisense | GAGAGCACCAUUCUCUCUAdTdT | (SEQ ID NO: 10) |
| M-37/57 sense | CCGAGGUCGAAACGUACGUdTdT | (SEQ ID NO: 11) |
| M-37/57 antisense | ACGUACGUUUCGACCUCGGdTdT | (SEQ ID NO: 12) |
| PB1-129/149 sense | CAGGAUACACCAUGGAUACdTdT | (SEQ ID NO: 13) |
| PB1-129/149 antisense | GUAUCCAUGGUGUAUCCUGdTdT | (SEQ ID NO: 14) |

SiRNA delivery. siRNAs (30 or 60 μg of GFP-949, NP-1496, or PA-2087) were incubated with jetPEI™ for oligonucleotides cationic polymer transfection reagent, N/P ratio=5 (Qbiogene, Inc., Carlsbad, Calif.; Cat. No. GDSP20130; N/P refers to the number of nitrogen residues per nucleotide phosphate in the jetPEI reagent) or with poly-L-lysine (MW (vis) 52,000; MW (LALLS) 41,800, Sigma Cat. No. P2636) for 20 min at room temperature in 5% glucose. The mixture was injected into mice intravenously, into the retro-orbital vein, 200 μl per mouse, 4 mice per group. 200 μl 5% glucose was injected into control (no treatment) mice. The mice were anesthetized with 2.5% Avertin before siRNA injection or intranasal infection.

Viral infection. B6 mice (maintained under standard laboratory conditions) were intranasally infected with PR8 virus by dropping virus-containing buffer into the mouse's nose with a pipette, 30 μl (12,000 pfu) per mouse.

Determination of viral titer. Mice were sacrificed at various times following infection, and lungs were harvested. Lungs were homogenized, and the homogenate was frozen and thawed twice to release virus. PR8 virus present in infected lungs was titered by infection of MDCK cells. Flat-bottom 96-well plates were seeded with 3×10⁴ MDCK cells per well, and 24 hrs later the serum-containing medium was removed. 25 μl of lung homogenate, either undiluted or diluted from 1×10⁻¹ to 1×10⁻⁷, was inoculated into triplicate wells. After 1 h incubation, 175 μl of infection medium with 4 μg/ml of trypsin was added to each well. Following a 48 h incubation at 37° C., the presence or absence of virus was determined by hemagglutination of chicken RBC by supernatant from infected cells. The hemagglutination assay was carried out in V-bottom 96-well plates. Serial 2-fold dilutions of supernatant were mixed with an equal volume of a 0.5% suspension (vol/vol) of chicken erythrocytes (Charles River Laboratories) and incubated on ice for 1 h. Wells containing an aadherent, homogeneous layer of erythrocytes were scored as positive. The virus titers were determined by interpolation of the dilution end point that infected 50% of wells by the method of Reed and Muench (TCID₅₀). The data from any two groups were compared by Student t test, which was used throughout the experiments described herein to evaluate significance.

Results

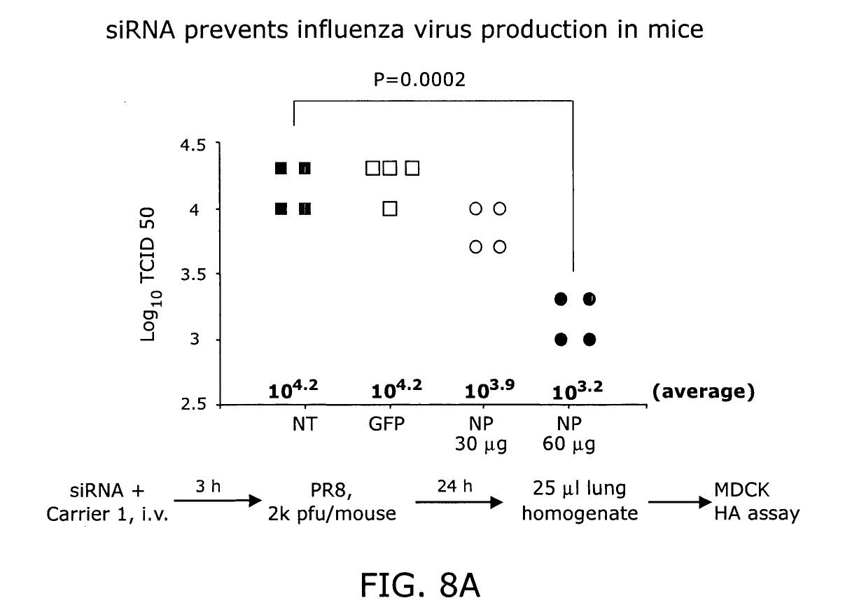

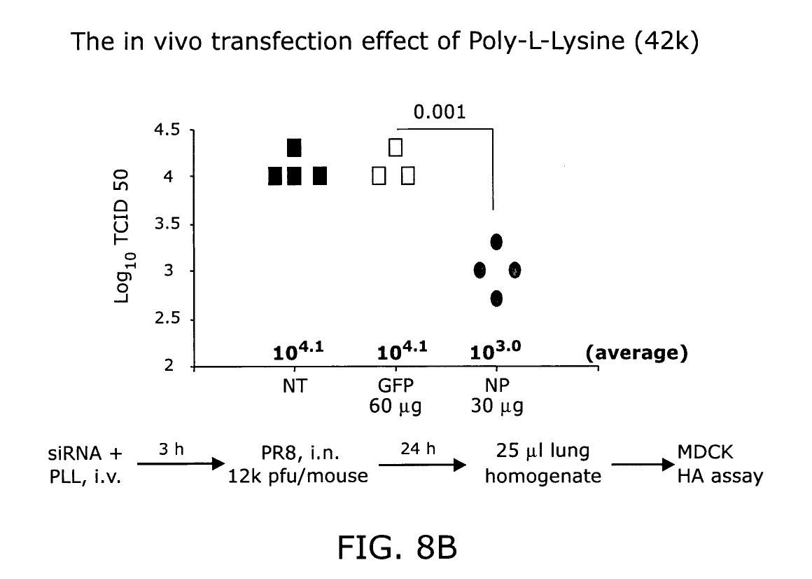

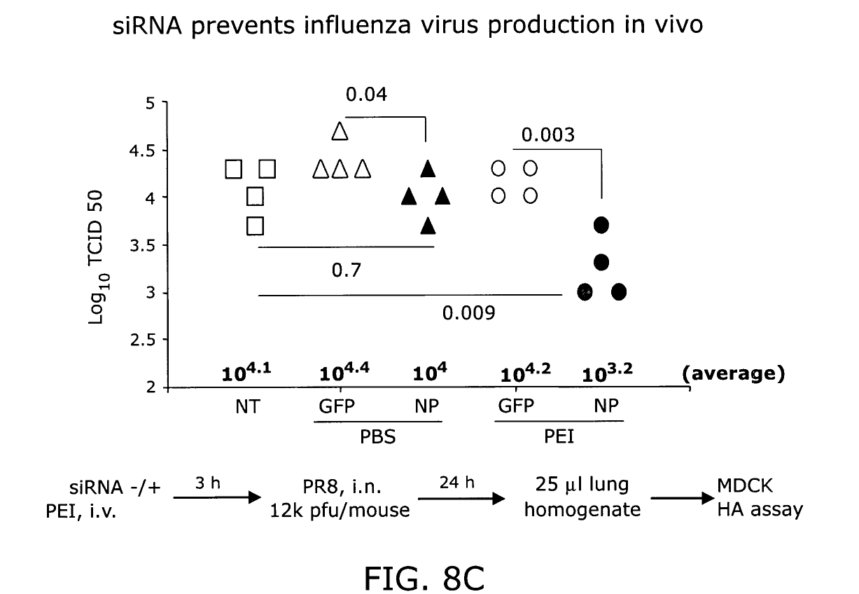

FIG. 8A shows results of an experiment demonstrating that siRNA targeted to viral NP transcripts inhibits influenza virus production in mice when administered prior to infection. 30 or 60 μg of GFP-949 or NP-1496 siRNAs were incubated with jetPEI and injected intravenously into mice as described above in Materials and Methods. Three hours later mice were intranasally infected with PR8 virus, 12000 pfu per mouse. Lungs were harvested 24 hours after infection. As shown in FIG. 8A, the average log₁₀TCID₅₀ of the lung homogenate for mice that received no siRNA treatment (NT; filled squares) or received an siRNA targeted to GFP (GFP 60 μg; open squares) was 4.2. In mice that were pretreated with 30 μg siRNA targeted to NP (NP 30 μg; open circles) and jetPEI, the average log₁₀TCID₅₀ of the lung homogenate was 3.9. In mice that were pretreated with 60 μg siRNA targeted to NP (NP 60 μg; filled circles) and jetPEI, the average log₁₀TCID₅₀ of the lung homogenate was 3.2. The difference in virus titer in the lung homogenate between the group that received no treatment and the group that received 60 μg NP siRNA was significant with P=0.0002. Data for individual mice are presented in Table 1 (NT=no treatment).

FIG. 8B shows results of another experiment demonstrating that siRNA targeted to viral NP transcripts inhibits influenza virus production in mice when administered prior to infection. 30 or 60 μg of GFP-949 or NP-1496 siRNAs were incubated with PLL and injected intravenously into mice as described above in Materials and Methods. Three hours later mice were intranasally infected with PR8 virus, 12000 pfu per mouse. Lungs were harvested 24 hours after infection. As shown in FIG. 8B, the average log₁₀TCID₅₀ of the lung homogenate for mice that received no siRNA treatment (NT; filled squares) or received an siRNA targeted to GFP (GFP 60 μg; open squares) was 4.1. In mice that were pretreated with 60 μg siRNA targeted to NP (NP 60 μg; filled circles) and PLL, the average log₁₀TCID₅₀ of the lung homogenate was 3.0. The difference in virus titer in the lung homogenate between the group that received 60 μg GFP and the group that received 60 μg NP siRNA was significant with P=0.001. Data for individual mice are presented in Table 1 (NT=no treatment). These data indicate that siRNA targeted to the influenza NP transcript reduced the virus titer in the lung when administered prior to virus infection. They also indicate that mixtures of siRNAs with cationic polymers are effective agents for the inhibition of influenza virus in the lung when administered by intravenous injection, not requiring techniques such as hydrodynamic transfection.

TABLE 1
Inhibition of influenza virus production in mice by siRNA

| | |
|---|---|
| Treatment | log₁₀TCID50 |

| | | | | |
|---|---|---|---|---|
| NT (jetPEI experiment) | 4.3 | 4.3 | 4.0 | 4.0 |
| GFP (60 μg) + jetPEI | 4.3 | 4.3 | 4.3 | 4.0 |
| NP (30 μg) + jetPEI | 4.0 | 4.0 | 3.7 | 3.7 |
| NP (60 μg) + jetPEI | 3.3 | 3.3 | 3.0 | 3.0 |
| NT (PLL experiment) | 4.0 | 4.3 | 4.0 | 4.0 |
| GFP (60 μg) + PLL | 4.3 | 4.0 | 4.0 | (not done) |
| NP (60 μg) + PLL | 3.3 | 3.0 | 3.0 | 2.7 |

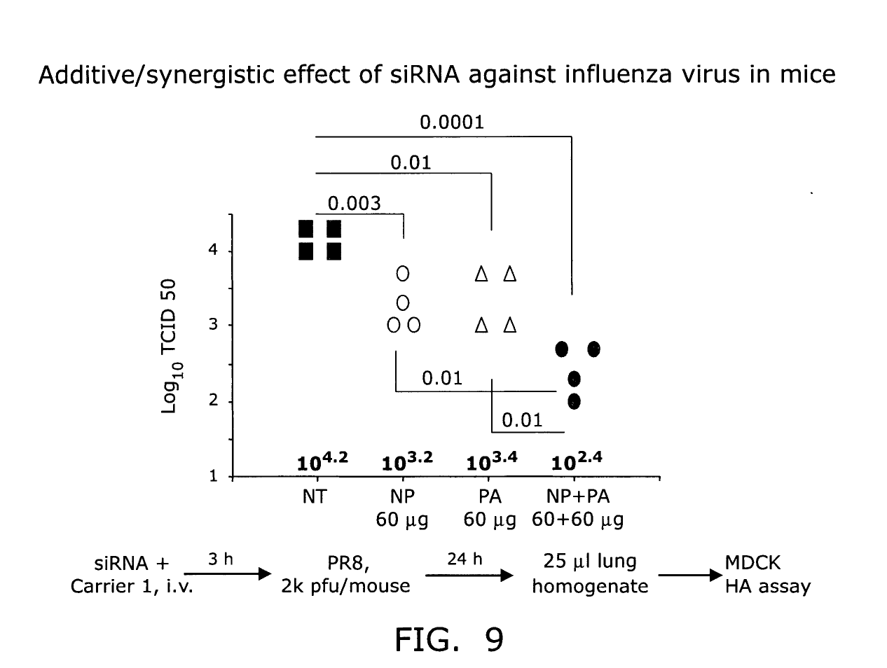

FIG. 9 shows results of an experiment demonstrating that siRNAs targeted to different influenza virus transcripts exhibit an additive effect. Sixty μg of NP-1496 siRNA, 60 μg PA-2087 siRNA, or 60 μg NP-1496 siRNA+60 μg PA-2087 siRNA were incubated with jetPEI and injected intravenously into mice as described above in Materials and Methods. Three hours later mice were intranasally infected with PR8 virus, 12000 pfu per mouse. Lungs were harvested 24 hours after infection. As shown in FIG. 9, the average log₁₀TCID₅₀ of the lung homogenate for mice that received no siRNA treatment (NT; filled squares) was 4.2. In mice that received 60 μg siRNA targeted to NP (NP 60 μg; open circles), the average log₁₀TCID₅₀ of the lung homogenate was 3.2. In mice that received 60 μg siRNA targeted to PA (PA 60 μg; open triangles), the average log₁₀TCID₅₀ of the lung homogenate was 3.4. In mice that received 60 μg siRNA targeted to NP+60 μg siRNA targeted to PA (NP+PA; filled circles), the average log₁₀TCID₅₀ of the lung homogenate was 2.4. The differences in virus titer in the lung homogenate between the group that received no treatment and the groups that received 60 μg NP siRNA, 60 μg PA siRNA, or 60 μg NP siRNA+60 μg PA siRNA were significant with P=0.003, 0.01, and 0.0001, respectively. The differences in lung homogenate between the groups that received 60 μg NP siRNA or 60 μg NP siRNA and the group that received 60 μg NP siRNA+60 μg PA siRNA were significant with P=0.01. Data for individual mice are presented in Table 2 (NT=no treatment). These data indicate that pretreatment with siRNA targeted to the influenza NP or PA transcript reduced the virus titer in the lungs of mice subsequently infected with influenza virus. The data further indicate that a combination of siRNA targeted to different viral transcripts exhibit an additive effect, suggesting that therapy with a combination of siRNAs targeted to different transcripts may allow a reduction in dose of each siRNA, relative to the amount of a single siRNA that would be needed to achieve equal efficacy. It is possible that certain siRNAs targeted to different transcripts may exhibit synergistic effects (i.e., effects that are greater than additive). The systematic approach to identification of potent siRNAs and siRNA combinations may be used to identify siRNA compositions in which siRNAs exhibit additive or synergistic effects.

TABLE 2
Additive effect of siRNA against influenza virus in mice

| | |
|---|---|
| Treatment | log₁₀TCID50 |

| | | | | |
|---|---|---|---|---|
| NT | 4.3 | 4.3 | 4.0 | 4.0 |
| NP (60 μg) | 3.7 | 3.3 | 3.0 | 3.0 |
| PA (60 μg) | 3.7 | 3.7 | 3.0 | 3.0 |
| NP + PA (60 μg | 2.7 | 2.7 | 2.3 | 2.0 |
| each) | | | | |

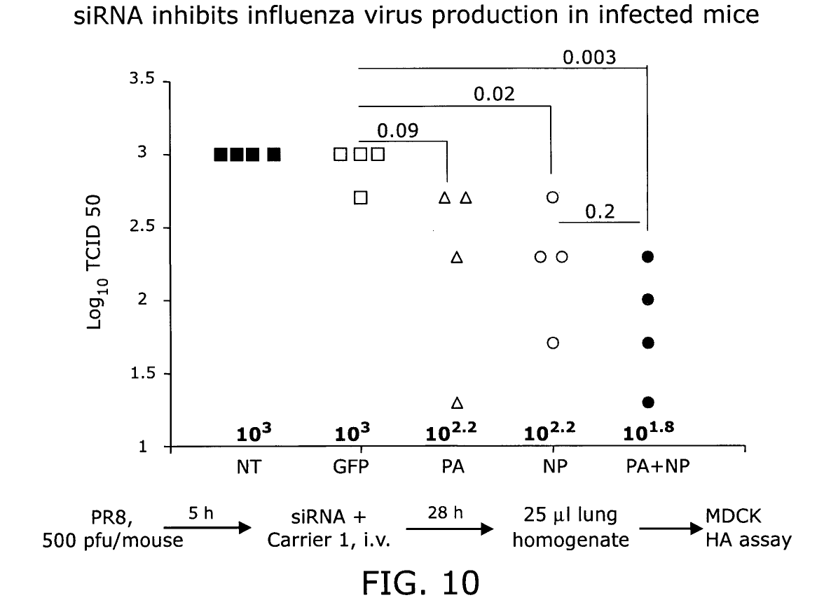

FIG. 10 shows results of an experiment demonstrating that siRNA targeted to viral NP transcripts inhibits influenza virus production in mice when administered following infection. Mice were intranasally infected with PR8 virus, 500 pfu. Sixty μg of GFP-949 siRNA, 60 μg PA-2087 siRNA, 60 μg NP-1496 siRNA, or 60 μg NP siRNA+60 μg PA siRNA were incubated with jetPEI and injected intravenously into mice 5 hours later as described above in Materials and Methods. Lungs were harvested 28 hours after administration of siRNA. As shown in FIG. 10, the average log₁₀TCID₅₀ of the lung homogenate for mice that received no siRNA treatment (NT; filled squares) or received the GFP-specific siRNA GFP-949 (GFP; open squares) was 3.0. In mice that received 60 μg siRNA targeted to PA (PA 60 μg; open triangles), the average log₁₀TCID₅₀ of the lung homogenate was 2.2. In mice that received 60 μg siRNA targeted to NP (NP 60 μg; open circles), the average log₁₀TCID₅₀ of the lung homogenate was 2.2. In mice that received 60 μg NP siRNA+60 μg PA siRNA (PA+NP; filled circles), the average log₁₀TCID₅₀ of the lung homogenate was 1.8. The differences in virus titer in the lung homogenate between the group that received no treatment and the groups that received 60 μg PA, NP siRNA, or 60 μg NP siRNA+60 μg PA siRNA were significant with P=0.09, 0.02, and 0.003, respectively. The difference in virus titer in the lung homogenate between the group that received NP siRNA and PA+NP siRNAs had a P value of 0.2. Data for individual mice are presented in Table 3 (NT=no treatment). These data indicate that siRNA targeted to the influenza NP and/or PA transcripts reduced the virus titer in the lung when administered following virus infection.

TABLE 3
Inhibition of influenza virus production
in infected mice by siRNA

| | |
|---|---|
| Treatment | log₁₀TCID50 |

| | | | | |
|---|---|---|---|---|
| NT | 3.0 | 3.0 | 3.0 | 3.0 |
| GFP (60 μg) | 3.0 | 3.0 | 3.0 | 2.7 |
| PA (60 μg) | 2.7 | 2.7 | 2.3 | 1.3 |
| NP (60 μg) | 2.7 | 2.3 | 2.3 | 1.7 |
| NP + PA (60 μg | 2.3 | 2.0 | 1.7 | 1.3 |
| each) | | | | |

### EXAMPLE 5

### Inhibition of Influenza Virus Production in Cells by Administration of a DNA Vector from which siRNA Precursors (Short Hairpin RNAs) can be Transcribed

Materials and Methods

Cell culture. Vero cells were seeded in 24-well plates at 4×10⁵ cells per well in 1 ml of DMEM-10% FCS and were incubated at 37° C. under 5% CO₂.

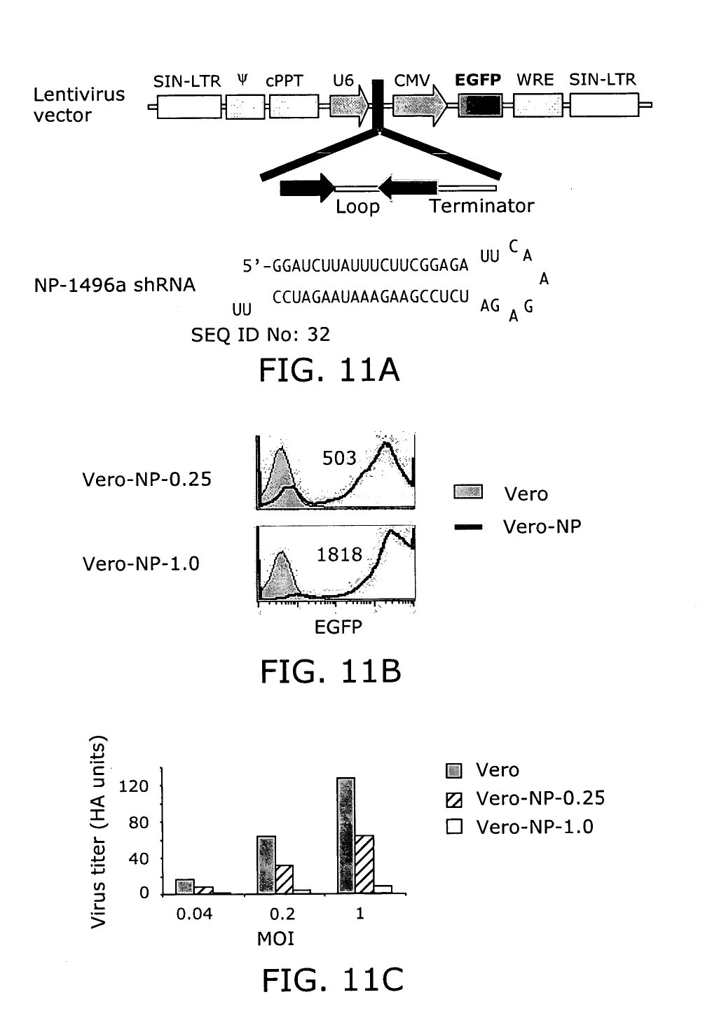

Production of lentivirus expressing shRNA. An oligonucleotide that serves as a template for synthesis of NP-1496a shRNA (see FIG. 1A) was cloned between the U6 promoter and termination sequence of lentiviral vector pLL3.7 (Rubinson, D., et al, Nature Genetics, Vol. 33, pp. 401-406, 2003), as depicted schematically in FIG. 11A. The oligonucleotide was inserted between the HpaI and XhoII restriction sites within the multiple cloning site of pLL3.7. This lentiviral vector also expresses EGFP for easy monitoring of transfected/infected cells. Lentivirus was produced by co-transfecting DNA vector encoding NP-1496a shRNA and packaging vectors into 293T cells. Forty-eight h later, culture supernatant containing lentivirus was collected, spun at 2000 rpm for 7 min at 4° C. and then filtered through a 0.45 um filter. Vero cells were seeded at 1×10⁵ per well in 24-well plates. After overnight culture, culture supernatants containing that contained the insert (either 0.25 ml or 1.0 ml) were added to wells in the presence of 8 ug/ml polybrene. The plates were then centrifuged at 2500 rpm, room temperature for 1 h and returned to culture. Twenty-four h after infection, the resulting Vero cell lines (Vero-NP-0.25, and Vero-NP-1.0) were analyzed for GFP expression by flow cytometry along with parental (non-infected) Vero cells. It is noted that NP-1496a differs from NP-1496 due to the inadvertent inclusion of an additional nucleotide (A) at the 3′ end of the sense portion and a complementary nucleotide (U) at the 5′ end of the antisense portion, resulting in a duplex portion that is 20 nt in length rather than 19 as in NP-1496. According to other embodiments of the invention NP-1496 sequences rather than NP-1496a sequences are used.

The sequences of the sense and antisense portions of NP-1496a were as follows:

| | | |
|---|---|---|
| NP-1496/1516a | GGAUCUUAUUUCUUCGGAGA | (SEQ ID NO: 15) |
| sense | | |
| NP-1496/1516a | UCUCCGAAGAAAUAAGAUCC | (SEQ ID NO: 16) |
| antisense | | |

The loop sequence for the NP-1496a hairpin was 5′-UUCAAGAGA-3′ (SEQ ID NO: 27)

Viral infection and determination of viral titer. Parental and NP-1496a shRNA expressing Vero cells were infected with PR8 virus at MOI of 0.04, 0.2 and 1. Virus titers in the supernatants were determined by hemagglutination (HA) assay 48 hrs after infection as described in Example 12.

Results

Lentivirus expressing NP-1496a shRNA were tested for ability to inhibit influenza virus production in Vero cells. The NP-1496a shRNA includes two self-complementary regions capable of forming a stem-loop structure containing a double-stranded portion that has the same sequence as the NP-1496a siRNA described above. Incubation of lentivirus-containing supernatants with Vero cells overnight resulted in expression of EGFP, indicating infection of Vero cells by lentivirus (FIG. 11B). When 1 ml of supernatant was used, almost all cells became EGFP positive and the mean fluorescence intensity was high (1818) (Vero-NP-1.0). When 0.25 ml of supernatant was used, most cells (˜95%) were EGFP positive and the mean fluorescence intensity was lower (503) (Vero-NP-0.25).

Parental and lentivirus-infected Vero cells were then infected with influenza virus at MOI of 0.04, 0.2, and 0.1, and virus titers were assayed 48 hrs after influenza virus infection. With increasing MOI, the virus titers increased in the supernatants of parental Vero cell cultures (FIG. 11C). In contrast, the virus titers remained very low in supernatants of Vero-NP-1.0 cell cultures, indicating influenza virus production was inhibited in these cells. Similarly, influenza virus production in Vero-NP-0.25 cell cultures was also partially inhibited. The viral titers are presented in Table 4. These results suggest that NP-1496 shRNA expressed from lentivirus vectors can be processed into siRNA to inhibit influenza virus production in Vero cells. The extent of inhibition appears to be proportional to the extent of virus infection per cell (indicated by EGFP level).

TABLE 4
Inhibition of influenza virus production by
siRNA expressed in cells in tissue culture

| Cell Line | Viral Titer | | |
|---|---|---|---|
| Vero | 16 | 64 | 128 |
| Vero-NP-0.25 | 8 | 32 | 64 |
| Vero-NP-1.0 | 1 | 4 | 8 |

### EXAMPLE 6

### Inhibition of Influenza Production in Mice by Intranasal Administration of a DNA Vector from which siRNA Precursors can be Transcribed

Materials and Methods

Construction of plasmids that serves as template for shRNA. Construction of a plasmid from which NP-1496a shRNA is expressed in described in Example 5. Oligonucleotides that serve as templates for synthesis of PB1-2257 shRNA or RSV-specific siRNA were cloned between the U6 promoter and termination sequence of lentiviral vector pLL3.7 as described in Example 13 and depicted schematically in FIG. 11A for NP-1496 shRNA. The sequences of the oligonucleotides were as follows:

NP-1496a sense:

| | |
|---|---|
| 5′-TGGATCTTATTTCTTCGGAGATTCAAGAGAT | (SEQ ID NO: 17) |
| CTCCGAAGAAATAAGATCCTTTTTTC-3′ | |

NP-1496a antisense:

| | |
|---|---|
| 5′-TCGAGAAAAAAGGATCTTATTTCTTCGGAGA | (SEQ ID NO: 18) |
| TCTCTTGAATCTCCGAAGAAATAAGATCCA-3′ | |

PB1-2257 sense:

| | |
|---|---|
| 5′-TGATCTGTTCCACCATTGAATTCAAGAGATT | (SEQ ID NO: 19) |
| CAATGGTGGAACAGATCTTTTTTC-3′ | |

PB 1-2257 antisense

| | |
|---|---|
| 5′-TCGAGAAAAAAGATCTGTTCCACCATTGAAT | (SEQ ID NO: 20) |
| CTCTTGAATTCAATGGTGGAACAGATCA-3′ | |

RSV sense:

| | |
|---|---|
| 5′-TGCGATAATATAACTGCAAGATTCAAGAGAT | (SEQ ID NO: 21) |
| CTTGCAGTTATATTATCGTTTTTTC-3′ | |

RSV antisense:

| | |
|---|---|
| 5′-TCGAGAAAAAACGATAATATAACTGCAAGAT | (SEQ ID NO: 22) |
| CTCTTGAATCTTGCAGTTATATTATCGCA-3′ | |

The RSV shRNA expressed from the vector comprising the above oligonucleotide is processed in vivo to generate an siRNA having sense and antisense strands with the following sequences:

| | | |
|---|---|---|
| Sense: | 5′-CGATAATATAACTGCAA | (SEQ ID NO: 23) |
| | GA-3′ | |
| Antisense: | 5′-TCTTGCAGTTATATTAT | (SEQ ID NO: 24) |
| | CG-3′ | |

A PA-specific hairpin may be similarly constructed using the following oligonucleotides:

PA-2087 sense:

| | |
|---|---|
| 5′-TGCAATTGAGGAGTGCCTGATTCAAGAGATC | (SEQ ID NO: 25) |
| AGGCACTCCTCAATTGCTTTTTTC-3′ | |

PA-2087 antisense:

| | |
|---|---|
| 5′-TCGAGAAAAAAGCAATTGAGGAGTGCCTGA | (SEQ ID NO: 26) |
| TCTCTTGAATCAGGCACTCCTCAATTGCA-3′ | |

Viral infection and determination of viral titer. These were performed as described in Example 12.

DNA Delivery. Plasmid DNAs capable of serving as templates for expression of NP-1496a shRNA, PB 1-2257, or RSV-specific shRNA (60 μg each) were individually mixed with 40 μl Infasurf® (ONY, Inc., Amherst N.Y.) and were administered intranasally to groups of mice, 4 mice each group, as described above. Sixty μl of 5% glucose was administered to the mouse in the no treatment (NT) group. The mice were intranasally infected with PR8 virus, 2000 pfu per mouse, 13 hours later, as described above. Lungs were harvested and viral titer determined 24 hours after infection.

Results

The ability of shRNAs expressed from DNA vectors to inhibit influenza virus infection in mice was tested. For these experiments, plasmid DNA was mixed with Infasurf, a natural surfactant extract from calf lung and that is known to promote DNA transfection in the lung (74). The DNA/Infasurf mixtures were instilled into mice by dropping the mixture into the nose using a pipette. Mice were infected with PR8 virus, 2000 pfu per mouse, 13 hours later. Twenty-four hrs after influenza virus infection, lungs were harvested and virus titers were measured by MDCK/hemagglutinin assay.

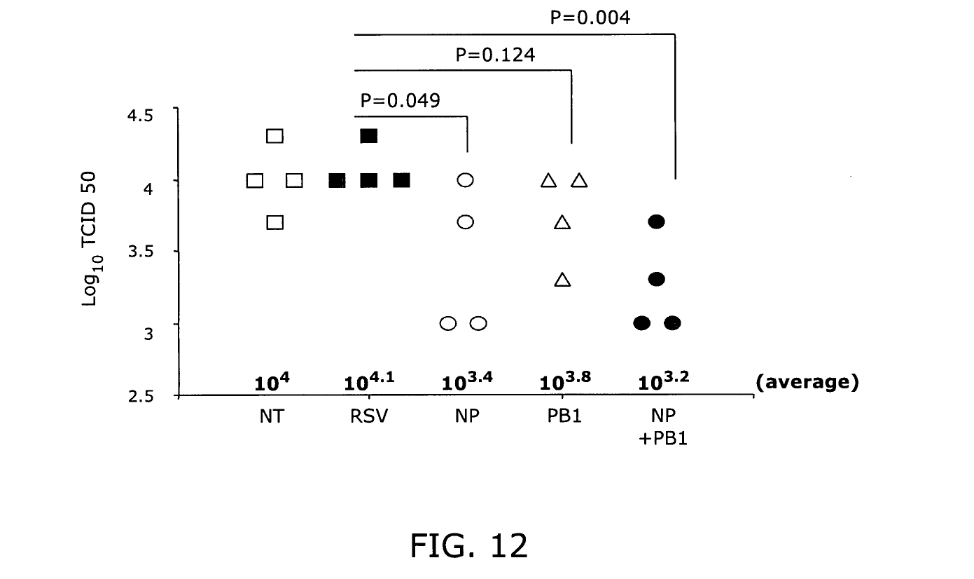

As shown in FIG. 12, virus titers were high in mice that were not given any plasmid DNA or were given a DNA vector expressing a respiratory syncytial virus (RSV)-specific shRNA. Lower virus titers were observed when mice were given plasmid DNA that expresses either NP-1496a shRNA or PB1-2257 shRNA. The virus titers were more significantly decreased when mice were given both influenza-specific plasmid DNAs together, one expressing NP-1496a shRNA and the other expressing PB 1-2257 shRNA. The average log₁₀TCID₅₀ of the lung homogenate for mice that received no siRNA treatment (NT; open squares) or received a plasmid encoding an RSV-specific siRNA (RSV; filled squares) was 4.0 or 4.1, respectively. In mice that received plasmid capable of serving as a template for NP-1496 shRNA (NP; open circles), the average log₁₀TCID₅₀ of the lung homogenate was 3.4. In mice that received plasmid capable of serving as a template for PB1-2257 shRNA (PB; open triangles), the average log₁₀TCID₅₀ of the lung homogenate was 3.8. In mice that received plasmids capable of serving as templates for NP and PB shRNAs (NP+PB1; filled circles), the average log₁₀TCID₅₀ of the lung homogenate was 3.2. The differences in virus titer in the lung homogenate between the group that received no treatment or RSV-specific shRNA plasmid and the groups that received NP shRNA plasmid, PB1 shRNA plasmid, or NP and PB1 shRNA plasmids had P values of 0.049, 0.124, and 0.004 respectively. Data for individual mice are presented in Table 5 (NT=no treatment). Preliminary experiments involving intranasal administration of NP-1496 siRNA in the presence of PBS or jetPEI but in the absence of Infasurf did not result in effective inhibition of influenza virus. These data indicate that siRNA targeted to the influenza NP and/or PB1 transcripts reduced the virus titer in the lung when administered following virus infection. These results show that shRNA intermediates expressed from DNA vectors can be processed into siRNA to inhibit influenza virus production in mice and demonstrate that Infasurf is a suitable delivery agent for the delivery of plasmids from which shRNA can be expressed.

TABLE 5
Inhibition of influenza virus production by
siRNA expressed in mice

| | |
|---|---|
| Treatment | log₁₀TCID50 |

| | | | | |
|---|---|---|---|---|
| NT | 4.3 | 4.0 | 4.0 | 4.3 |
| RSV (60 μg) | 4.3 | 4.0 | 4.0 | 4.0 |
| NP (60 μg) | 4.0 | 3.7 | 3.0 | 3.0 |
| PB1 (60 μg) | 4.0 | 4.0 | 3.7 | 3.3 |
| NP + PB1 (60 μg | 3.7 | 3.3 | 3.0 | 3.0 |
| each) | | | | |

### EXAMPLE 7

### Cationic Polymers Promote Cellular Uptake of siRNA

Materials and Methods

Reagents. Poly-L-lysines of two different average molecular weights [poly-L-lysine (MW (vis) 52,000; MW (LALLS) 41,800, Cat. No. P2636) and poly-L-lysine (MW (vis) 9,400; MW (LALLS) 8,400, Cat. No. P2636], poly-L-arginine (MW 15,000-70,000 Cat. No. P7762) and 3-(4,5-dimethylthiazol-2-yl)-2,5-diphenyl tetrazolium bromide (MTT) were purchased from Sigma. For purposes of description molecular weights obtained using the LALLS method will be assumed, but it is to be understood that molecular weights are approximate since the polymers display some heterogeneity in size.

Gel retardation assay. siRNA-polymer complexes were formed by mixing 10 μl of siRNA (10 pmol in 10 mM Hepes buffer, pH 7.2) with 10 μl of polymer solution containing varying amounts of polymer. Complexes were allowed to form for 30 min at room temperature, after which 20 μl was run on a 4% agarose gel. Bands were visualized with ethidium-bromide staining.

Cytotoxicity assay. siRNA-polymer complexes were formed by mixing equal amounts (50 pmol) of siRNA in 10 mM Hepes buffer, pH 7.2 with polymer solution containing varying amounts of polymer for 30 min at room temperature. Cytotoxicity was evaluated by MTT assay. Cells were seeded in 96-well plates at 30,000 cells per well in 0.2 ml of DMEM containing 10% fatal calf serum (FCS). After overnight incubation at 37° C., the medium was removed and replaced with 0.18 ml OPTI-MEM (GIBCO/BRL). siRNA-polymer complexes in 20 μl of Hepes buffer were added to the cells. After a 6-h incubation at 37° C., the polymer-containing medium was removed and replaced with DMEM-10% FCS. The metabolic activity of the cells was measured 24 h later using the MTT assay according to the manufacturer's instructions. Experiments were performed in triplicate, and the data was averaged.

Cell culture, transfection, siRNA-polymer complex formation, and viral titer determination. Vero cells were grown in DMEM containing 10% heat-inactivated FCS, 2 mM L-glutamine, 100 units/ml penicillin, and 100 μg/ml streptomycin at 37° C. under a 5% CO2/95% air atmosphere. For transfection experiments, logarithmic-phase Vero cells were seeded in 24-well plates at 4×10⁵ cells per well in 1 ml of DMEM-10% FCS. After overnight incubation at 37° C., siRNA-polymer complexes were formed by adding 50 μl of siRNA (400 pmol (about 700 ng) in 10 mM Hepes buffer, pH 7.2) to 50 μl of polymer vortexing. Different concentrations of polymer were used in order to achieve complete complex formation between the siRNA and polymer. The mixture was incubated at room temperature for 30 min to complete complex formation. The cell-growth medium was removed and replaced with OPTI-MEM I (Life Technologies) just before the complexes were added.

After incubating the cells with the complexes for 6 h at 37° C. under 5% CO₂, the complex-containing medium was removed and 200 μl of PR8 virus in infection medium, MOI=0.04, consisting of DMEM, 0.3% BSA (Sigma), 10 mM Hepes, 100 units/ml penicillin, and 100 μg/ml streptomycin, was added to each well. After incubation for 1 h at room temperature with constant rocking, 0.8 ml of infection medium containing 4 μg/ml trypsin was added to each well and the cells were cultured at 37° C. under 5% CO₂. At different times after infection, supernatants were harvested from infected cultures and the virus titer was determined by hemagglutination (HA) assay as described above.

Transfection of siRNA by Lipofectamine 2000 (Life Technology) was carried out according to the manufacturer's instruction for adherent cell lines. Briefly, logarithmic-phase Vero cells were seeded in 24-well plate at 4×10⁵ cells per well in 1 ml of DMEM-10% FCS and were incubated at 37° C. under 5% CO₂. On the next day, 50 μl of diluted Lipofectamine 2000 in OPTI-MEM I were added to 50 μl of siRNA (400 pmol in OPTI-MEM I) to form complexes. The cell were washed and incubated with serum-free medium. The complexes were applied to the cells and the cells were incubated at 37° C. for 6 h before being washed and infected with influenza virus as described above. At different times after infection, supernatants were harvested from infected cultures and the virus titer was determined by hemagglutination (HA) assay as described above.

Results

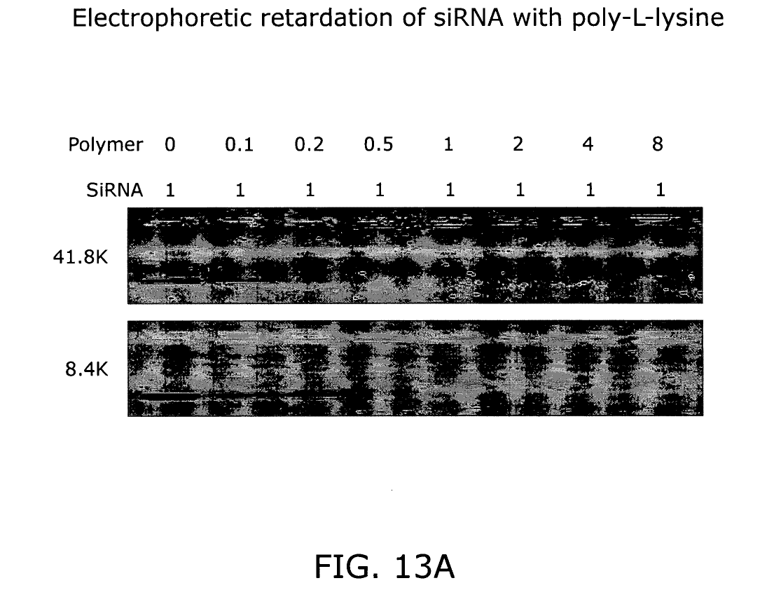

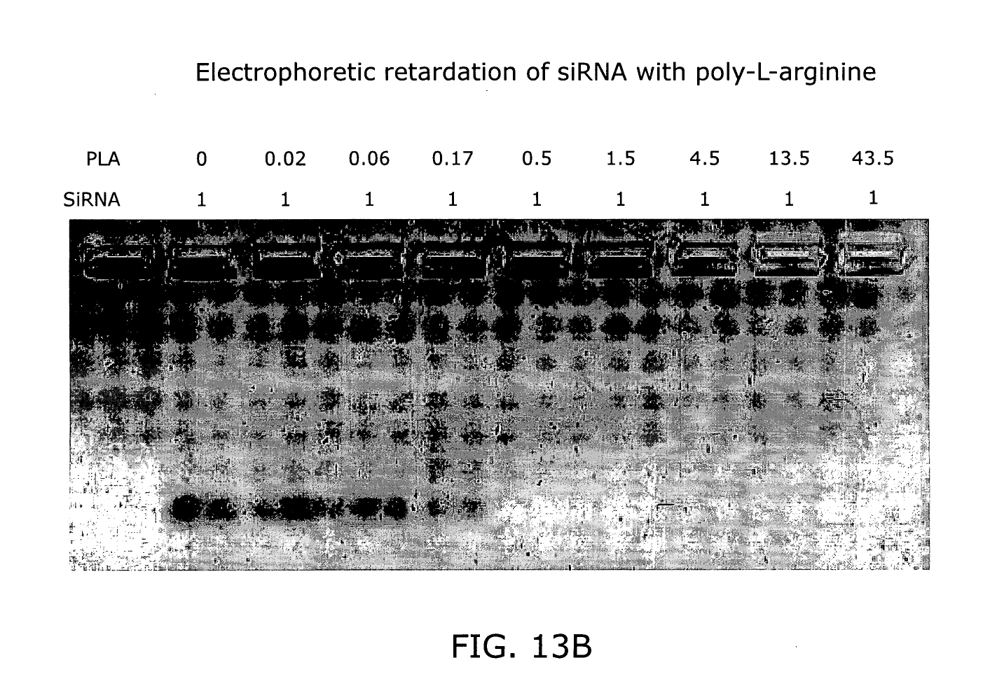

The ability of poly-L-lysine (PLL) and poly-L-arginine (PLA) to form complexes with siRNA and promote uptake of siRNA by cultured cells was tested. To determine whether PLL and/or PLA form complexes with siRNA, a fixed amount of NP-1496 siRNA was mixed with increasing amounts of polymer. Formation of polymer/siRNA complexes was then visualized by electrophoresis in a 4% agarose gel. With increasing amounts of polymer, electrophoretic mobility of siRNA was retarded (FIGS. 13A and 13B), indicating complex formation. FIGS. 13A and 13B represent complex formation between siRNAs and PLL (41.8K) or PLA, respectively. The amount of polymer used in each panel increases from left to right. In FIGS. 13A and 13B in each panel, a band can be seen in the lanes on the left, indicating lack of complex formation and hence entry of the siRNA into the gel and subsequent migration. As one moves to the right, the band disappears, indicating complex formation and failure of the complex to enter the gel and migrate.

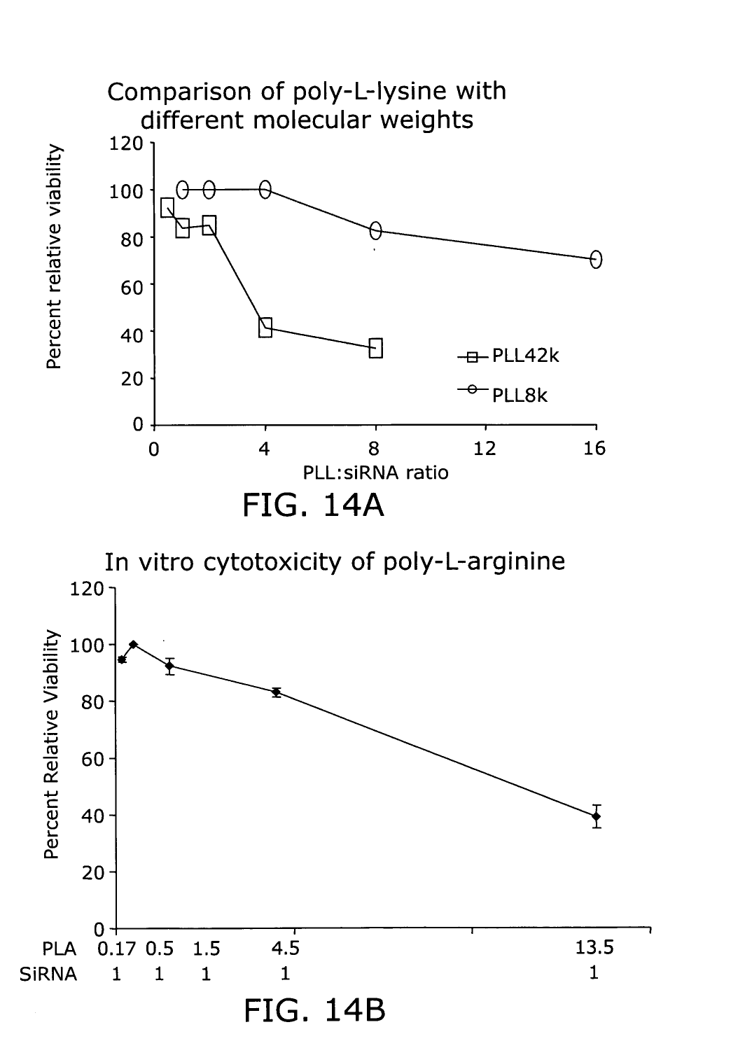

To investigate cytotoxicity of siRNA/polymer complexes, mixtures of siRNA and PLL or PLA at different ratios were added to Vero cell cultures in 96-well plates. The metabolic activity of the cells were measured by MTT assay (74). Experiments were performed in triplicate, and data was averaged. Cell viability was significantly reduced with increasing amounts of PLL (MW ˜42K) whereas PLL (˜8K) showed significantly lower toxicity, exhibiting minimal or no toxicity at PLL/siRNA ratios as high as 4:1 (FIG. 14A; circles=PLL (MW˜8K); squares=PLL (MW˜42K)). Cell viability was reduced with increasing PLA/siRNA ratios as shown in FIG. 14B, but viability remained above 80% at PLA/siRNA ratios as high as 4.5:1. The polymer/siRNA ratio is indicated on the x-axis in FIGS. 14A and 14B. The data plotted in FIGS. 14A and 14B are presented in Tables 6 and 7. In Table 6 the numbers indicate % viability of cells following treatment with polymer/siRNA complexes, relative to % viability of untreated cells. ND=Not done. In Table 7 the numbers indicate PLA/siRNA ratio, % survival, and standard deviation as shown.

TABLE 6
Cytotoxicity of PLL/siRNA complexes (% survival)

| | polymer/siRNA ratio | | | | | |
|---|---|---|---|---|---|---|
| Treatment | 0.5 | 1.0 | 2.0 | 4.0 | 8.0 | 16.0 |
| PLL ˜8.4 K | 92.26 | 83.57 | 84.39 | 41.42 | 32.51 | ND |
| PLL ˜41.8 K | ND | 100 | 100 | 100 | 82.55 | 69.63 |

TABLE 7
Cytotoxicity of PLA/siRNA complexes (% survival)

| | polymer/siRNA ratio | | | | |
|---|---|---|---|---|---|
| | 0.17 | 0.5 | 1.5 | 4.5 | 13.5 |
| % survival | 94.61 | 100 | 92.33 | 83 | 39.19 |
| Standard deviation | .74 | 1.91 | 2.92 | 1.51 | 4.12 |

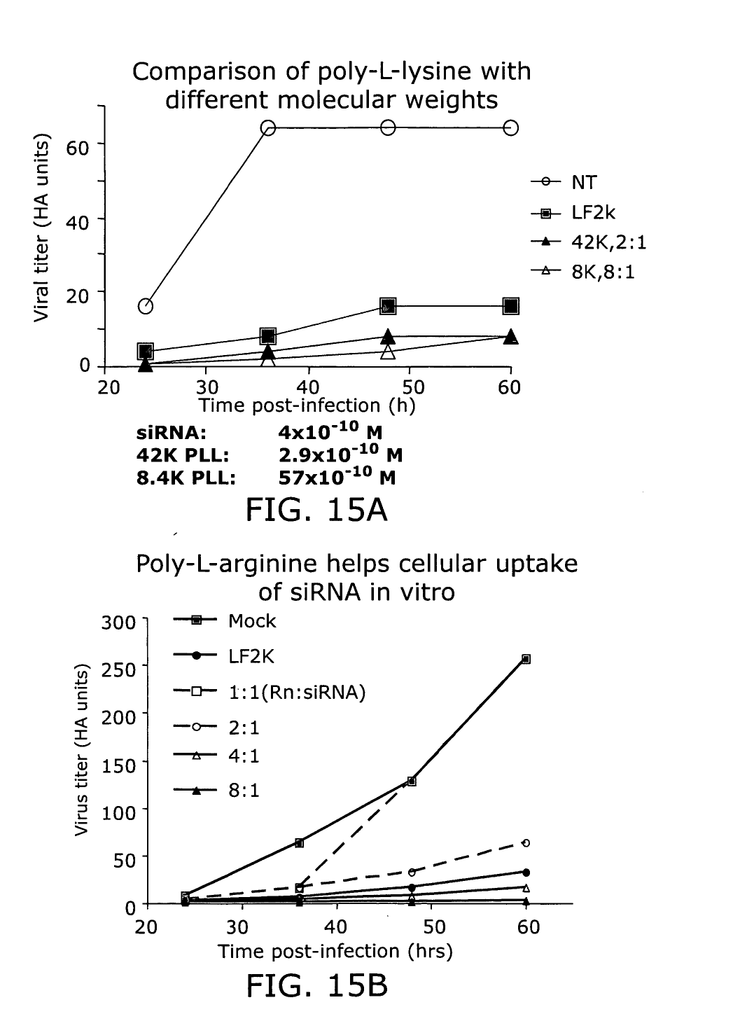

To determine whether PLL or PLA promotes cellular uptake of siRNA, various amounts of polymer and NP-1496 were mixed at ratios at which all siRNA was complexed with polymer. Equal amounts of siRNA were used in each case. A lower polymer/siRNA ratio was used for ˜42K PLL than for ˜8K PLL since the former proved more toxic to cells. The complexes were added to Vero cells, and 6 hrs later the cultures were infected with PR8 virus. At different times after infection, culture supernatants were harvested and assayed for virus by HA assay. FIG. 15A is a plot of virus titers over time in cells receiving various transfection treatments (circles=no treatment; squares=Lipofectamine; filled triangles=PLL (˜42K at PLL/siRNA ratio=2); open triangles=PLL (˜8K at PLL/siRNA ratio=8). As shown in FIG. 15A, virus titers increased with time in the non-transfected cultures. Virus titers were significantly lower in cultures that were transfected with NP-1496/Lipofectamine and were even lower in cultures treated with PLL/NP-1496 complexes. The data plotted in FIG. 15A are presented in Table 8 (NT=no treatment; LF2K=Lipofectamine. The PLL:siRNA ratio is indicated in parentheses.

PLA was similarly tested over a range of polymer/siRNA ratios. FIG. 15B is a plot of virus titers over time in cells receiving various transfection treatments (filled squares=mock transfection; filled circles=Lipofectamine; open squares=PLA at PLA/siRNA ratio=1; open circles=PLA at PLA/siRNA ratio=2; open triangles=PLA at PLA/siRNA ratio=4; filled triangles=PLA at PLA/siRNA ratio=8). As shown in FIG. 15B, virus titers increased with time in the control (mock-transfected) culture and in the culture treated with PLA/siRNA at a 1:1 ratio. Virus titers were significantly lower in cultures that were transfected with NP-1496/Lipofectamine and were even lower in cultures treated with PLA/siRNA complexes containing complexes at PLA/siRNA ratios of 4:1 or higher. Increasing amounts of polymer resulted in greater reduction in viral titer. The data plotted in FIG. 15B are presented in Table 9.

TABLE 8
Inhibition of influenza virus production by
polymer/siRNA complexes

Time (hours)

| Treatment | 24 | 36 | 48 | 60 |
|---|---|---|---|---|
| mock transfection | 16 | 64 | 64 | 64 |
| LF2K | 4 | 8 | 16 | 16 |
| PLL ˜42 K (2:1) | 1 | 4 | 8 | 8 |
| PLL ˜8 K (8:1) | 1 | 2 | 4 | 8 |

TABLE 9
Inhibition of influenza virus production by
polymer/siRNA complexes

Time (hours)

| Treatment | 24 | 36 | 48 | 60 |
|---|---|---|---|---|
| mock transfection | 8 | 64 | 128 | 256 |
| LF2K | 2 | 6 | 16 | 32 |
| PLA (1:1) | 4 | 16 | 128 | 256 |
| PLA (2:1) | 4 | 16 | 32 | 64 |
| PLA (4:1) | 1 | 4 | 8 | 16 |
| PLA (8:1) | 1 | 1 | 1 | 2 |

Thus, cationic polymers promote cellular uptake of siRNA and inhibit influenza virus production in a cell line and are more effective than the widely used transfection reagent Lipofectamine. These results also suggest that additional cationic polymers may readily be identified to stimulate cellular uptake of siRNA and describe a method for their identification. PLL and PLA can serve as positive controls for such efforts.

### EXAMPLE 8

### siRNAs that Target Viral RNA Polymerase or Nucleoprotein Inhibit Influenza A Virus Production in Chicken Embryos

Materials and Methods

SiRNA-oligofectamine complex formation and chicken embryo inoculation. SiRNAs were prepared as described above. Chicken eggs were maintained under standard conditions. 30 μl of Oligofectamine (product number: 12252011 from Life Technologies, now Invitrogen) was mixed with 30 μl of Opti-MEM I (Gibco) and incubated at RT for 5 min. 2.5 mmol (10 μl) of siRNA was mixed with 30 μl of Opti-MEM I and added into diluted oligofectamine. The siRNA and oligofectamine was incubated at RT for 30 min. 10-day old chicken eggs were inoculated with siRNA-oligofectamine complex together with 100 μl of PR8 virus (5000 pfu/ml). The eggs were incubated at 37° C. for the indicated time and allantoic fluid was harvested. Viral titer in allantoic fluid was tested by HA assay as described above.

Results

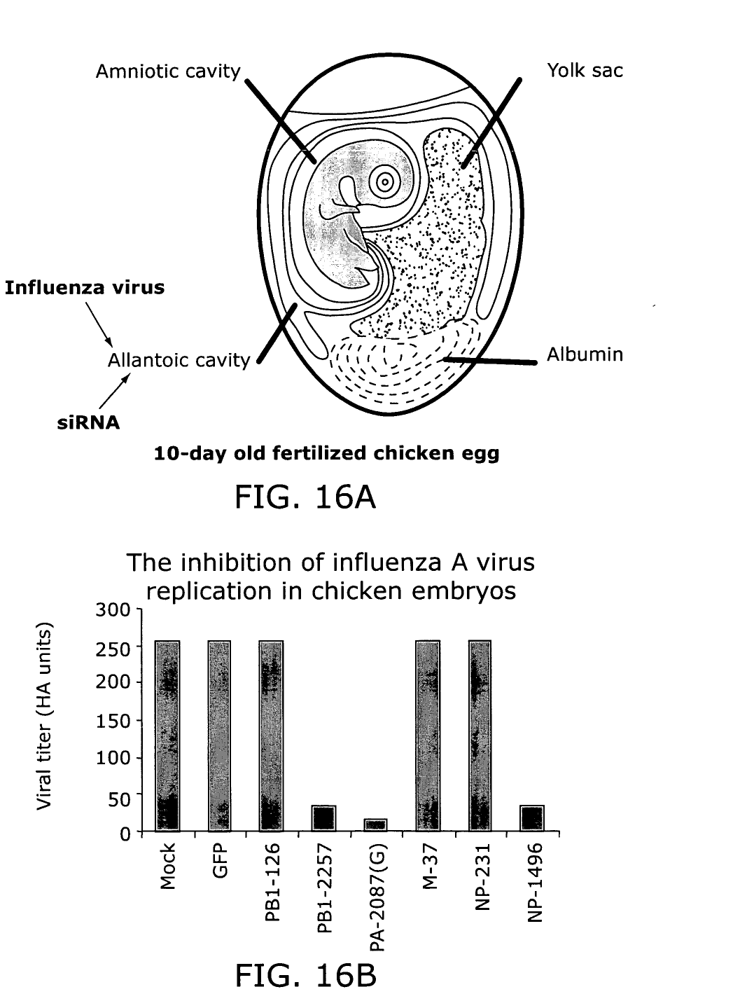

The ability of siRNA-containing compositions to inhibit influenza virus production in fertilized chicken eggs was assayed. Oligofectamine, a lipid-based agent that has been shown to facilitate intracellular uptake of DNA oligonucleotides as well as siRNAs in cell culture was used (25). Briefly, PR8 virus alone (500 pfu) or virus plus siRNA-oligofectamine complex was injected into the allantoic cavity of 10-day old chicken eggs as shown schematically in FIG. 16A. Allantoic fluids were collected 17 hours later for measuring virus titers by hemagglutinin assay. As shown in FIG. 16B, when virus was injected alone (in the presence of Oligofectamine), high virus titers were readily detected. Co-injection of GFP-949 did not significantly affect the virus titer. No significant reduction in virus titer was observed when Oligofectamine was omitted, indicating the utility of employing this delivery agent for achieving inhibition using siRNA in an intact avian organism.

The injection of siRNAs specific for influenza virus showed results consistent with those observed in MDCK cells (described in co-pending patent application “Influenza Therapeutic” filed on even date herewith). The same siRNAs (NP-1496, PA2087 and PB1-2257) that inhibited influenza virus production in MDCK cells also inhibited virus production in chicken eggs, whereas the siRNAs (NP-231, M-37 and PB 1-129) that were less effective in MDCK cells were ineffective in fertilized chicken eggs. Thus, siRNAs are also effective in interfering with influenza virus production in fertilized chicken eggs when delivered in combination with a lipid-based carrier.

### REFERENCES

1. Lamb, R. A., and R. M. Krug. 2001. Orthomyxoviridae: the viruese and their replication. Fundamental Virology Ed. D. M. Knipe & P. M. Hpwley:725-770.
2. Simonsen, L., K. Fukuda, L. B. Schomberger, and N. J. Cox. 2000. The impact of influenza epidemics on hospitalizations. J. Infect. Dis. 181:831-837.
3. Webster, R. G., W. J. Bean, O. T. Gorman, T. M. Chambers, and Y. Kawaoka. 1992. Evolution and ecology of influenza A viruses. Microbiol. Rev. 56:152-179.
4. Parvin, J. D., A. Moscona, W. T. Pan, J. M. Leider, and P. Palese. 1986. Measurement of the mutation rates of animal viruses: influenza A virus and poliovirus type 1. J. Virol. 59:377-383.
5. Smith, F. L., and P. Palese. 1989. Variation in influenza virus genes:epidemiology, pathogenic, and evolutionary consequences. The influenza viruses Krug, R. M. ed.:New York:Plenum.
6. Webster, R. G., W. G. Laver, G. M. air, and S. G. C. 1982. Molecular mechanisms of variation in influenza viruses. Nature 296:115-121.
7. Webby, R. J., and R. G. Webster. 2001. Emergence of influenza A viruses. Phil. Trans. R. Soc. Lond. 356:1817-1828.
8. Patterson, K. D., and G. F. Pyle. 1991. The geography and mortality of the 1918 influenza pandemic. Bull. Hist. Med. 65:4-21.
9. Taubenberger, J. K., A. H. Reid, T. A. Janczewski, and T. G. GFanning. 2001. Integrating historical, clinical and molecular genetic data in order to explain the origin and virulence of the 1918 Spanish influenza virus. Phil. Trans. R. Soc. Lond. 356:1829-1839.
10. Claas, E. C., A. D. Osterhaus, R. van Beek, J. C. De Jong, G. F. Rimmelzwaan, D. A. Senne, S. Krauss, K. F. Shortridge, and R. G. Webster. 1998. Human influenza A H5N1 virus related to a highly pathogenic avian influenza virus. Lancet 351:472-477.
11. Yuen, K. Y., P. K. Chan, M. Peiris, D. N. Tsang, T. L. Que, K. F. Shortridge, P. T. Cheung, W. K. To, E. T. Ho, R. Sung, and A. F. Cheng. 1998. Clinical features and rapid viral diagnosis of human disease associated with avian influenza A H5N1 virus. Lancet 351:467-471.
12. Fukuda, F., C. B. Bridges, and T. L.e.a. Brammer. 1999. Prevention and control of influenza: recommendations of the advisory committee on immunization practices (ACIP). MMWR Morb. Mortal. Wkly. Rep. 48:1-28.
13. Castle, S. C. 2000. Clinical relevane of age-related immune dysfunction. Clin. Infect. Dis. 31:578-585.
14. Luscher-Mattli, M. 2000. Influenza chemotherapy: a review of the present state of art and of new drugs in development. Arch. Virol. 145:2233-2248.
15. Cox, N. J., and K. Subbarao. 1999. Influenza. Lancet 354:1277-1282.
16. Vaucheret, H., C. Beclin, and M. Fagard. 2001. Post-transcriptional gene silencing in plants. J. Cell Sci. 114:3083-3091.
17. Sharp, P. A. 2001. RNA interference-2001. Genes Dev. 15:485-490.
18. Brantl, S. 2002. Antisense-RNA regulation and RNA interference. Biochem. Biophy. Acta 1575:15-25.
19. Baulcombe, D. 2002. RNA silencing. Curr. Biol. 12:R82-R84.
20. Fire, A., S. Xu, M. K. Montgomery, S. A. Kostas, S. E. Driver, and M. C. C. 1998. Potent and specific genetic interference by double-stranded RNA in Caenorhabditis elegans. Nature 391:806-811.
21. Elbashir, S., J. Harborth, W. Lendeckel, A. Yalcin, K. Weber, and T. Tuschl. 2001. Duplexes of 21-nucleotide RNAs mediate RNA interference in cultured mammalian cells. Nature 411:494-498.
22. McManus, M. T., and P. A. Sharp. 2002. Gene silencing in mammals by short interfering RNAs. Nature Rev. Gene. 3:737-747.
23. Kumar, M., and G. G. Carmichael. 1998. Antisense RNA: function and fate of duplex RNA in cells of higher eukaryotes. Microbiol. Mol. Biol. Rev. 62:1415-1434.
24. Gitlin, L., S. Karelsky, and R. Andino. 2002. Short interfering RNA confers intracellular antiviral immunity in human cells. Nature 418:430-434.
25. Pderoso de Lima, M. C., S. Simoes, P. Pires, H. Faneca, and N. Duzgunes. 2001. Cationic lipid-DNA complexes in gene delivery: from biophysics to biological applications. Adv. Drug Deliv. Rev. 47:277-294.
26. Holen, T., M. Amarzguioui, M. T. Wiiger, E. Babaie, and H. Prydz. 2002. Positional effects of short interfering RNAs targeting the human coagulation trigger tissue factor. Nucleic Acids Res. 30:1757-1766.
27. McManus, M. T., B. B. Haines, C. P. Dillon, C. E. Whitehurst, L. Van Parijs, J. Chen, and P. A. Sharp. 2002. siRNA-mediated gene silencing in T-lymphocytes. J. Immunol. in press.
28. Elbashir, S. M., J. Martinez, A. Patkaniowska, W. Lendeckel, and T. Tuschl. 2001. Functional anatomy of siRNAs for mediating efficient RNAi in Drosophila melanogaster embryo lysate. EMBO J. 20:6877-6888.
29. Yang, D., H. Lu, and J. W. Erickson. 2000. Evidence that processed small dsRNAs may mediate sequence-specific mRNA degradation during RNAi in Drosophila embryos. Curr. Biol. 10:1191-1200.
30. Caplen, N. J., J. Fleenor, A. Fire, and R. A. Morgan. 2000. dsRNA-mediated gene silencing in cultured Drosophila cells: a tissue culture model for the analysis of RAN interference. Gene 252:95-105.
31. Edwards, D. A., J. Hanes, G. Caponetti, J. Hrkach, A. Ben-Jebria, M. L. Eskew, J. Mintzes, D. Deaver, N. Lotan, and R. Langer. 1997. Large porous particles for pulmonary drug delivery. Science 276:1868-1871.
32. Putnam, D., C. A. Gentry, D. W. Pack, and R. Langer. 2001. Polymer-based gene delivery with low cytotoxicity by a unique balance of side-chain termini. Proc. Natl. Acad. Sci. USA 98:1200-1205.
33. Lynn, D. M., and R. Langer. 2000. Degradable Poly(-amino esters): Synthesis, Characterization, and Self-Assembly with Plasmid DNA. J. Am. Chem. Soc. 122:10761-10768.
34. Lynn, D. M., D. G. Anderson, D. Putnam, and R. Langer. 2001. Accelerated discovery of synthetic transfection vectors: parallel synthesis and screening of a degrable polymer library. J. Am. Chem. Soc. 123:8155-8156.
35. Han, S.-O., R. I. Mahato, Y. K. Sung, and S. W. Kim. 2000. Development of Biomaterials for gene therapy. Mol. Therapy 2:302-317.
36. Soane, R. J., M. Frier, A. C. Perkins, N. S. Jones, S. S. Davis, and L. Illum. 1999. Evaluation of the clearance characteristics of bioadhesive systems in humans. Int. J. Pharm. 178:55-65.
37. Boussif, O., F. Lezoualc'h, M. A. Zanta, M. D. Mergny, D. Scherman, B. Demeneix, and J. P. Behr. 1995. A versatile vector for gene and oligonucleotide transfer into cells in culture and in vivo: polyethylenimine. Proc. Natl. Acad. Sci. USA 92:7297-7301.
38. Astafieva, I., I. Maksimova, E. Lukanidin, V. Alakhov, and A. Kabanov. 1996. Enhancement of the polycation-mediated DNA uptake and cell transfection with Pluronic P85 block copolymer. FESB Lett. 389:278-280.
39. Davis, S. S. 1999. Delivery of peptide and non-peptide drugs through the respiratory tract. Pharm. Sci. Technol. Today 2:450-457.
40. Roy, K., H.-Q. Mao, S.-K. Huang, and K. W. Leong. 1999. Oral delivery with chitosan/DNA nanoparticles generates immunologic protection in murine model of peanut allergy. Nat. Med. 5:387-391.
41. Hansen, M. B., S. E. Nielsen, and K. Berg. 1989. Re-examination and further development of a precise and rapid dye method for measuring cell growth/cell kill. J. Immunol. Methods 119:203-210.
42. Green, M., and P. M. Loewenstein. 1988. Autonomous functional domains of chemically synthesized human immunodeficiency virus tat trans-activator protein. Cell 55:1179-1188.
43. Frankel, A. D., and C. O. Pabo. 1988. Cellular uptake of the tat protein from human immunodeficiency virus. Cell 55:1189-1193.
44. Elliott, G., and P. O'Hare. 1997. Intercellular trafficking and protein delivery by a herpesvirus structural protein. Cell 88:223-233.
45. Joliot, A., C. Pernelle, H. Deagostini-Bazin, and A. Prochiantz. 1991. Antennapedia homeobox peptide regulates neural morphogenesis. Proc. Natl. Acad. Sci. USA 88:1864-1868.
46. Fawell, S., J. Seery, Y. Daikh, C. Moore, L. L. Chen, B. Pepinsky, and J. Barsoum. 1994. Tat-mediated delivery of heterologous proteins into cells. Proc. Natl. Acad. Sci. USA 91:664-668.
47. Schwarze, S. R., A. Ho, A. Vocero-Akbani, and S. F. Dowdy. 1999. In vivo protein transduction: delivery of a biologically active protein. Science 285:1569-1572.
48. Derossi, D., G. Chassaing, and A. Prochiantz. 1998. Trojan peptides: the penetratin system for intracellular delivery. Trends Cell Biol. 8:84-87.
49. Troy, C. M., D. Derossi, A. Prochiantz, L. A. Greene, and M. L. Shelanski. 1996. Downregulation of Cu/Zn superoxide dismutase leads to cell death via the nitric oxide-peroxynitrite pathway. J. Neurosci. 16:253-261.
50. Allinquant, B., P. Hantraye, P. Mailleux, K. Moya, C. Bouillot, and A. Prochiantz. 1995. Downregulation of amyloid precursor protein inhibits neurite outgrowth in vitro. J. Cell Biol. 128:919-927.
51. Futaki, S., T. Suzuki, W. Ohashi, T. Yagami, S. Tanaka, K. Ueda, and Y. Sugiura. 2001. Arginine-rich peptides. An abundant source of membrane-permeable peptides having potential as carriers for intracellular protein delivery. J. Biol. Chem. 276:5836-5840.
52. Densmore, C. L., F. M. Orson, B. Xu, B. M. Kinsey, J. C. Waldrep, P. Hua, B. Bhogal, and V. Knight. 1999. Aerosol delivery of robust polyethyleneimine-DNA complexes for gene therapy and genetic immunization. Mol. Therapy 1:180-188.
53. Arppe, J., M. Widgred, and J. C. Waldrep. 1998. Pulmonary pharmacokinetics of cyclosporin A liposomes. Intl. J. Pharm. 161:205-214.
54. Griesenbach, U., A. chonn, R. Cassady, V. Hannam, C. Ackerley, M. Post, A. K. Transwell, K. Olek, H. O'Brodovich, and L.-C. Tsui. 1998. Comparison between intratracheal and intravenous administration of liposome-DNA complexes for cystic fibrosis lung gene therapy. Gene Ther. 5:181-188.
55. Orson, F. M., L. Song, A. Gautam, C. L. DEnsmore, B. Bhogal, and B. M. Kinsey. 2002. Gene delivery to the lung using protein/polyethyleneimine/plasmid complexes. Gene Therapy 9:463-471.
56. Gautam, A., C. L. Densmore, E. Golunski, B. Xu, and J. C. Waldrep. 2001. Transgene expression in mouse airway epithelium by aerosol gene therapy with PEI-DNA complexes. Mol. Therapy 3:551-556.
57. Tabata, Y., and Y. Ikada. 1988. Effect of size and surface charge of polymer microspheres on their phagocytosis by macrophage. J. Biomed. Mater. Res. 22:837-842.
58. Vanbever, R., J. D. Mintzes, J. Wang, J. Nice, D. chen, R. Batycky, L. R., and D. A. Edwards. 1999. Formulation and physical characterization of large porous particles for inhalation. Pharmaceutical Res. 16:1735-1742.
59. McManus, M. T., C. P. Peterson, B. B. Haines, J. Chen, and P. A. Sharp. 2002. Gene silencing using micro-RNA designed hairpins. RNA 8:842-850.
60. Myslinski, E., J. C. Ame, A. Krol, and P. Carbon. 2001. An unusually compact external promoter for RNA polymerase III transcription of the human H1 RNA gene. Nucleic Acids Res. 29:2502-2509.
61. Brummelkamp, T. R., R. Bernards, and R. Agami. 2002. A system for stable expression of short interfering RNAs in mammalian cells. Science 296:550-553.
62. Paddison, P. J., A. A. Caudy, E. Bernstein, G. J. Hannon, and D. S. Conklin. 2002. Short hairpin RNAs (shRNAs) induce sequence-sepcific silencing in mammalian cells. Genes Dev. 16:948-958.
63. Gil, J., and M. Esteban. 2000. Induction of apoptosis by the dsRNA-dependent protein kinase (PKR): mechanism of action. Apoptosis 5:107-114.
64. Bitko, V., and S. Barik. 2001. Phenotypic silencing of cytoplasmic genes using sequence-specific double-stranded short interfering RNA and its application in the reverse genetic of wild type negative-strand RNA viruses. BMC Microbiol. 1:34-43.
65. Garcia-Sastre, A. (2002) Microbes & Inf 4, 647-655.
66. Katze, M. G., He, Y. & Gale Jr., M. (2002) Nature Rev. Immunol. 2, 675-687.
67. Diaz, M. O., Ziemin, S., Le Beau, M. M., Pitha, P., Smith, S. D., Chilcote, R. R. & Rowley, J. D. (1988) Proc. Natl. Acad. Sci. USA 85, 5259-5263.
68. Diaz, M. O., Pomykala, H. M., Bohlander, S. K., Maltepe, E., Malik, K., Brownstein, B. & Olopade, O. I. (1994) Genomics 22, 540-552.
69. Kim, M.-J., Latham, A. G. & Krug, R. M. (2002) Proc. Natl. Acad. Sci. USA 99, 10096-10101.
70. Medcalf, L., Poole, E., Elton, D. & Digard, P. (1999) J. Virol. 73, 7349-7356.
71. Shapiro, G. I. & Krug, R. M. (1988) J. Virol. 62, 2285-2290.
72. Beaton, A. R. & Krug, R. M. (1986) Proc. Natl. Acad. Sci. USA 83, 6282-6286
73. Lois, C., E. J. Hong, S. Pease, E. J. Brown, and D. Baltimore. (2002) Science 295:868-872.
74. Weiss, D. J., G. M. Mutlu, L. Bonneau, M. Mendez, Y. Wang, V. Dumasius, and P. Factor. (2002) Mol. Ther. 6:43-49.
75. Hansen, M. B., S. E. Nielsen, and K. Berg. (1989) J. Immunol. Methods 119:203-210.
76. Kunath, K, von Harpe A, Fischer D, Petersen H, Bickel U, Voigt K, Kissel T. (2003) J Control Release 89(1): 113-25.
77. Jobe A. Surfactant treatment for respiratory distress syndrome. Respir Care 1986;31(6): 467-476.
78. Berry D. Neonatology in the 1990's: surfactant replacement therapy becomes a reality. Clin Pediatr 1991;30(3): 167-170.
79. Avery M E, Mead J. Surface properties in relation to atelectasis and hyaline membrane disease. Am J Dis Child 1959;97:517-523.
80. von Neergard K. Neue Auffassungen über einen Grundbegriff der Atemmechanik: die Retraktionskraft der Lunge, äbhangig von der Oberfläichenspannung in den Alveolen. Z Ges Exp Med 1929;66:373.
81. Hallman M, Teramo K, Ylikorkala O, Merritt T A. Natural surfactant substitution in respiratory distress syndrome. J Perinat Med 1987;15:463-468.
82. Bloom B T, Kattwinkel J, Hall R T, et al. Comparison of Infasurf (calf lung surfactant extract) to Survanta (beractant) in the treatment and prevention of respiratory distress syndrome. Pediatrics. 1997;100:31-38.
83. Mizuno K, Ikegami M, Chen C-M, et al. Surfactant protein-B supplementation improves in vivo function of a modified natural surfactant. Pediatr Res. 1995;37:271-276.
84. Hall S B, Venkitaraman A R, Whitsett J A, et al. Importance of hydrophobic apoproteins as constituents of clinical exogenous surfactants. Am Rev Respir Dis. 1992;145:24-30.
85. C. H. Ahn, S. Y. Chae, Y. H. Bae and S. W. Kim, Biodegradable poly(ethylenimine) for plasmid DNA delivery. J Control Release 80, 273-82, 2002.
86. Kichler, A., Leborgne C, Coeytaux E, Danos O. Polyethylenimine-mediated gene delivery: a mechanistic study. J Gene Med. 2001 March-April; 3(2): 135-44.
87. Brissault B, Kichler A, Guis C, Leborgne C, Danos O, Cheradame H. Synthesis of linear polyethylenimine derivatives for DNA transfectionBioconjug Chem. 2003 May-June;14(3):581-7
88. Kichler A, Leborgne C, Marz J, Danos O, Bechinger B.Histidine-rich amphipathic peptide antibiotics promote efficient delivery of DNA into mammalian cellsProc Natl Acad Sci USA. 2003 Feb. 18;100(4):1564-8
Brantl, S. (2002). Antisense-RNA regulation and RNA interference. Biochim Biophys Acta 1575, 15-25.
90. Semizarov, D., Frost, L., Sarthy, A., Kroeger, P., Halbert, D. N., and Fesik, S. W. (2003). Specificity of short interfering RNA determined through gene expression signatures. Proc Natl Acad Sci USA 100, 6347-6352.
91. Chi, J. T., Chang, H. Y., Wang, N. N., Chang, D. S., Dunphy, N., and Brown, P. O. (2003). Genomewide view of gene silencing by small interfering RNAs. Proc Natl Acad Sci USA 100, 6343-6346.
92. Bitko, V., and Barik, S. (2001). Phenotypic silencing of cytoplasmic genes using sequence-specific double-stranded short interfering RNA and its application in the reverse genetics of wild type negative-strand RNA viruses. BMC Microbiol 1, 34.
93. Anderson, D. G., Lynn, D. M., and Langer, R. (2003). Semi-Automated Synthesis and Screening of a Large Library of Degradable Cationic Polymers for Gene Delivery. Angew Chem Int Ed Engl 42, 3153-3158.
94. Ge, Q., McManus, M., Nguyen, T., Shen, C.-H., Sharp, P. A., Eisen, H. N., and Chen, J. (2003). RNA interference of influenza virus production by directly targeting mRNA for degradation and indirectly inhibiting all viral RNA transcription. Proc Natl Acad Sci USA 100, 2718-2723.
95. Kumar, M., and Carmichael, G. G. (1998). Antisense RNA: function and fate of duplex RNA in cells of higher eukaryotes. Microbiol Mol Biol Rev 62, 1415-1434.
96. Lèuscher-Mattli, M. (2000). Influenza chemotherapy: a review of the present state of art and of new drugs in development. Arch Virol 145, 2233-2248.
97. McCaffrey, A. P., Meuse, L., Pham, T. T., Conklin, D. S., Hannon, G. J., and Kay, M. A. (2002). RNA interference in adult mice. Nature 418, 38-39.
98. McCaffrey, A. P., Nakai, H., Pandey, K., Huang, Z., Salazar, F. H., Xu, H., Wieland, S. F., Marion, P. L., and Kay, M. A. (2003). Inhibition of hepatitis B virus in mice by RNA interference. Nat Biotechnol 21, 639-644.
99. Rubinson, D. A., Dillon, C. P., Kwiatkowski, A. V., Sievers, C., Yang, L., Kopinja, J., Rooney, D. L., Ihrig, M. M., McManus, M. T., Gertler, F. B., et al. (2003). A lentivirus-based system to functionally silence genes in primary mammalian cells, stem cells and transgenic mice by RNA interference. Nat Genet 33, 401-406.
100. Shapiro, G. I., and Krug, R. M. (1988). Influenza virus RNA replication in vitro: synthesis of viral template RNAs and virion RNAs in the absence of an added primer. J Virol 62, 2285-2290.
101. Shen, C., Buck, A. K., Liu, X., Winkler, M., and Reske, S. N. (2003). Gene silencing by adenovirus-delivered siRNA. FEBS lett 539, 111-114.
102. Simeoni, F., Morris, M. C., Heitz, F., and Divita, G. (2003). Insight into the mechanism of the peptide-based gene delivery system MPG: implications for delivery of siRNA into mammalian cells. Nucleic Acids Res 31, 2717-2724.
103. Soane, R. J., Frier, M., Perkins, A. C., Jones, N. S., Davis, S. S., and Illum, L. (1999). Evaluation of the clearance characteristics of bioadhesive systems in humans. Int J Pharm 178, 55-65.
104. Stiver, G. (2003). The treatment of influenza with antiviral drugs. CMAJ 168, 49-56.
105. Tachibana, R., Harashima, H., Ide, N., Ukitsu, S., Ohta, Y., Suzuki, N., Kikuchi, H., Shinohara, Y., and Kiwada, H. (2002). Quantitative analysis of correlation between number of nuclear plasmids and gene expression activity after transfection with cationic liposomes. Pharm Res 19, 377-381.
106. Thomas, C. E., Ehrhardt, A., and Kay, M. A. (2003). Progress and problems with the use of viral vectors for gene therapy. Nat Rev Genet 4, 346-358.
107. Xia, H., Mao, Q., Paulson, H. L., and Davidson, B. L. (2002). siRNA-mediated gene silencing in vitro and in vivo. Nat Biotechnol 20, 1006-1010.
108. Zhang, G., Song, Y. K., and Liu, D. (2000). Long-term expression of human alpha1-antitrypsin gene in mouse liver achieved by intravenous administration of plasmid DNA using a hydrodynamics-based procedure. Gene Ther 7, 1344-1349
109. Cheung, M., and Lieberman, J. M. (2002). Influenza: update on strategies for managment. Contemporay Pediatrics 19, 82-114.
110. Yang, P., Althage, A., Chung, J., and Chisari, F. (2000) Hydrodynamic injection of viral DNA: A mouse model of acute hepatitis B virus infection, Proc. Natl. Acad. Sci., 99(21): 13825-13830.
111. Zhang, G., Budker, V., Wolff, J A (1999) High levels of foreign gene expression in hepatocytes after tail vein injections of naked plasmid DNA, Hum Gene Ther, 10:1735-1737.
112. Liu, F., Song, Y. K., Liu, D. (1999) Hydrodynamics-based transfection in animals by systemic administration of plasmid DNA. Gene Therapy; 6:1258-1266.
113. Courrier, H. M., Butz, N., and Vandamme, T. F. (2002), Pulmonary drug delivery systems: recent developments and prospects. Crit. Rev. Ther. Drug Carrier Syst. 19(4-5):425-98.
114. Agu, R., Ugwoke, M. I., Armand, M., Kinget, R., and Verbeke, N. (2001). The lung as a route for systemic delivery of therapeutic proteins and peptides, Respir. Res. 2:198-209.
115. Lewis, D. L., Hagstrom, J. E., Loomis, A. G., Wolff, J. A., and Herweijer, H. (2002). Efficient delivery of siRNA for inhibition of gene expression in postnatal mice, Nat. Genet. 32:107-108.
116. Amarzguioui, A., Holen, T., Babaie, E., and Prydz, H. Tolerance for mutations and chemical modifications in a siRNA. Nuc. Acids. Res. 31(2): 589-595 (2003).
117. Braasch, D. A., Jensen, S., Liu, Y., Kaur, K., Arar, K., White, M. A., and Corey, D. R. RNA Interference in Mammalian Cells by Chemically Modified RNA. Biochemistry 42: 7967-7975 (2003).
118. Chiu, Y-L. and Rana, T. M. siRNA function in RNAi: A chemical modification analysis. RNA 9(9): 1034-1048 (2003).
119. McCown, M., Diamond, M. S., and Pekosz, A., Virology, 313: 514-524 (2003).
120. Gratton, J. P., Yu, J., Griffith, J. W., Babbitt, R. W., Scotland, R. S., Hickey, R., Giordano, F. J., and Sessa, W. C., Cell-permeable peptides improve cellular uptake and therapeutic gene delivery of replication-deficient viruses in cells and in vivo. Nat. Med., 9(3): 357-362 (2003).
121. McKenzie, D. L., Kwok, K. Y., and Rice, K. G., A Potent New Class of Reductively Activated Peptide Gene Delivery Agents. J. Biol. Chem., 275(14): 9970-9977 (2000).
122. Park, Y., Kwok, K. Y., Boukarim, C., and Rice, K. G., Synthesis of Sulfhydryl Cross-Linking Poly(Ethylene Glycol Peptides and Glycopeptides as Carriers for Gene Delivery. Bioconjugate Chem., 13: 232-239 (2002).
123. Zhang, X., Sawyer, G., J., Dong, X., Qiu, Y., Collins, L., and Fabre, J. W. The in vivo use of chloroquine to promote non-viral gene delivery to the liver via the portal vein and bile duct. J. Gene Med., 5:209-218, 2003.
124. Thomas, N., and Klibanov, A. M. Non-viral gene therapy: polycation-mediated DNA delivery. Appl. Microbiol. Biotechnol. 62:27-34 (2003).
125. S.-O. Han, R. I. Mahato, Y. K. Sung, S. W. Kim, “Development of biomaterials for gene therapy”, Molecular Therapy 2:302317, 2000.
126. Weiss, D., Delivery of Gene Transfer Vectors to the Lung: Obstacles and the Role of Adjunct Techniques for Airway Administration. Mol. Therapy 6(2) (2002).
127. Ferrari S, Geddes D M, Alton E W. Barriers to and new approaches for gene therapy and gene delivery in cystic fibrosis. Adv Drug Deliv Rev, 54(11): 1373-93 (2002).
128. Orson F M, Song L, Gautam A, Densmore C L, Bhogal B S, Kinsey B M. Gene delivery to the lung using protein/polyethylenimine/plasmid complexes. Gene Ther. 2002 April; 9(7): 463-71.
129. Tiyaboonchai, W., Woiszwillo, J., and Middauth, C. R. Formulation and characterization of DNA-polyethylenemimine-dextran sulfate nanoparticles. Eur. J. Pharm. Sci. 19:191-202 (2003).
130. Benns, J., Mahato, R., and Kim, S. W., Optimization of factors influencing the transfection efficiency of folate-PEG-folate-graft polyethyleneimine, J. Cont. Release 79:255-269 (2002).
131. Putnam, D., Zelikin, A. N., Izumrudov, V. A., and Langer, R., Polyhistidine-PEG: DNA nanocomposites for gene delivery, Biomaterials 24: 4425-4433 (2003).
132. Benns, J., Choi, J-S., Mahato, R. I., Park, J-H., and Kim, S. W., pH-Sensitive Cationic Polymer Gene Delivery Vehicle: N-Ac-poly(L-histidine)-graft-poly(L-lysine) Comb Shaped Polymer, Bioconj. Chem. 11:637-645 (2000).
133. Panyam, J., Zhou, W-Z., Prabha, S., Sahoo, S. K., and Labhasetwar, V., Rapid endo-lysosomal escape of poly(DL-lactide-co-glycolide) nanoparticles: implications for drug and gene delivery, FASEB J., 16: 1217-1226 (2002).
134. Lindgren, M., Hallbrink, M., Prochiantz, A., and Langel, U. Cell-penetrating peptides. Trends Pharmacol. Sci. 21:99-103 (2000).
135. Morris, M. C., Depollier, J., Mery, J., Heitz, F., and Divita, G., A peptide carrier for the biologically active proteins into mammalian cells. Nat. Biotechnol. 19:1174-1176 (2001).
136. Schwarze, S. R. and Dowdy, S. F. In vivo protein transduction: intracellular delivery of biologically active proteins, compounds, and DNA. Trends Pharmacol. Sci. 21:99-103 (2000).
137. Amarzguioui, A., Holen, T., Babaie, E., and Prydz, H. Tolerance for mutations and chemical modifications in a siRNA. Nuc. Acids. Res. 31(2): 589-595 (2003).
138. Braasch, D. A., Jensen, S., Liu, Y., Kaur, K., Arar, K., White, M. A., and Corey, D. R>RNA Interference in Mammalian Cells by Chemically Modified RNA. Biochemistry 42: 7967-7975 (2003).
139. Chiu, Y-L. and Rana, T. M. siRNA function in RNAi: A chemical modification analysis. RNA 9(9): 1034-1048 (2003).
140. Ross, J. S., Fletcher, J. A., Linette, G. P., Stec, J., Clark, E., Ayers, M., Symmans, W. F., Pusztaie, L., and Kenneth J. Bloom, K. J. The HER-2/neu Gene and Protein in Breast Cancer 2003: Biomarker and Target of Therapy. The Oncologist, Vol. 8, No. 4, 307-325 (2003).
141. Satishchandran C. Characterization of a new class of DNA delivery complexe formed by the local anesthetic bupivacaine. Biochimica et Biophysica Acta 1468: 20-30 (2000).
142. Jeong J H, Park T G., Poly(L-lysine)-g-poly(D, L-lactic-co-glycolic acid) micelles for low cytotoxic biodegradable gene delivery carriers. J Control Release, 82(1): 159-66 (2002)

1. A composition comprising:
an RNAi-inducing entity, wherein the RNAi-inducing entity is targeted to a target transcript; and
a delivery agent selected from the group consisting of: cationic polymers, modified cationic polymers, peptide molecular transporters, surfactants suitable for introduction into the lung, liposomes, non-cationic polymers, modified non-cationic polymers, bupivacaine, and chloroquine.

2. The composition of claim 1, wherein the delivery agent comprises a delivery-enhancing moiety to enhance delivery to a cell of interest.

3. The composition of claim 2, wherein the delivery-enhancing moiety comprises an antibody, antibody fragment, or ligand that specifically binds to a molecule expressed by the cell of interest.

4. The composition of claim 3, wherein the cell of interest is a respiratory epithelial cell.

5. The composition of claim 2, wherein the delivery-enhancing moiety comprises a moiety selected to reduce degradation, clearance, or nonspecific binding of the delivery agent.

6. The composition of claim 1, wherein a disease or clinical condition, or a symptom thereof, is associated with excessive expression or inappropriate expression of the target transcript or inappropriate or excessive functional activity of a polypeptide encoded by the target transcript.

7. The composition of claim 1, wherein the RNAi-inducing entity comprises an siRNA.

8. The composition of claim 1, wherein the RNAi-inducing entity comprises an shRNA.

9. The composition of claim 1, wherein the RNAi-inducing entity comprises a lentivirus.

10. The composition of claim 1, wherein the RNAi-inducing entity comprises an RNAi-inducing vector.

11. The composition of claim 10, wherein:
the vector comprises a nucleic acid comprising a promoter for RNA polymerase III.

12. The composition of claim 11, wherein:
the promoter is a U6 or H1 promoter.

13. The composition of claim 1, wherein the RNAi-inducing entity comprises a viral vector.

14. The composition of claim 1, wherein the RNAi-inducing entity comprises a lentiviral vector.

15. The composition of claim 1, wherein the RNAi-inducing entity comprises a DNA vector.

16. The composition of claim 1, wherein:
the RNAi-inducing entity is an siRNA or shRNA targeted to a target transcript or an RNAi-inducing vector whose presence within a cell results in production of an siRNA or shRNA targeted to a target transcript, wherein the siRNA or shRNA comprises a portion that is perfectly complementary to a region of the target transcript, wherein the portion is at least 15 nucleotides in length.

17. The composition of claim 1, wherein:
the RNAi-inducing entity is an siRNA or shRNA targeted to a target transcript or an RNAi-inducing vector whose presence within a cell results in production of an siRNA or shRNA targeted to a target transcript, wherein the siRNA or shRNA comprises a portion that is perfectly complementary to a region of the target transcript, wherein the portion is approximately 19 nucleotides in length.

18. The composition of claim 1, wherein:
the RNAi-inducing entity is an siRNA or shRNA targeted to a target transcript or an RNAi-inducing vector whose presence within a cell results in production of an siRNA or shRNA targeted to a target transcript, wherein the siRNA or shRNA comprises a portion that is perfectly complementary to a portion of the target transcript, with the exception of three or fewer nucleotides, wherein the portion is at least 15 nucleotides in length.

19. The composition of claim 1, wherein:
the RNAi-inducing entity is an siRNA or shRNA targeted to a target transcript or an RNAi-inducing vector whose presence within a cell results in production of an siRNA or shRNA targeted to a target transcript, wherein the siRNA or shRNA comprises a portion that is perfectly complementary to a portion of the target transcript, with the exception of three or fewer nucleotides, wherein the portion is approximately 19 nucleotides in length.

20. The composition of claim 1, further comprising at least one pharmaceutically acceptable diluent, excipient, or carrier.

21. The composition of claim 1, wherein:
the composition comprises a plurality of different siRNAs, shRNAs, or RNAi-inducing vectors whose presence within a cell results in production of a plurality of different siRNAs or shRNAs, wherein the siRNAs or shRNAs are targeted to a single target transcript.

22. The composition of claim 1, wherein:
the composition comprises a plurality of different siRNAs, shRNAs, or RNAi-inducing vectors whose presence within a cell results in production of a plurality of different siRNAs or shRNAs, wherein the siRNAs or shRNAs are targeted to different target transcripts.

23. The composition of claim 1, wherein the delivery agent is selected from the group consisting of cationic polymers and modified cationic polymers.

24. The composition of claim 23, wherein the cationic polymer is selected from the group consisting of polylysine, polyarginine, polyethyleneimine, polyvinylpyrrolidone, chitosan, and poly(β-amino ester) polymers.

25. The composition of claim 24, wherein the cationic polymer is polyethyleneimine.

26. The composition of claim 24, wherein the cationic polymer is selected from the group consisting of poly(β-amino ester) polymers.

27. The composition of claim 24, wherein the modified cationic polymer incorporates a modification selected to reduce the cationic nature of the polymer.

28. The composition of claim 27, wherein the modification comprises substitution with a group selected from the group consisting of: acetyl, imidazole, succinyl, and acyl.

29. The composition of claim 24, wherein between 25% and 75% of the residues of the modified cationic polymer are modified.

30. The composition of claim 29, wherein approximately 50% of the residues of the modified cationic polymer are modified.

31. The composition of claim 23, wherein the RNAi-inducing entity comprises an siRNA.

32. The composition of claim 23, wherein the RNAi-inducing entity comprises an shRNA.

33. The composition of claim 23, wherein the RNAi-inducing entity comprises an RNAi-inducing vector.

34. The composition of claim 23, wherein the RNAi-inducing entity comprises a DNA vector.

35. The composition of claim 23, wherein the RNAi-inducing entity comprises a viral vector.

36. The composition of claim 23, wherein the RNAi-inducing entity comprises a lentiviral vector.

37. The composition of claim 23, wherein the RNAi-inducing entity comprises a lentivirus.

38. A method of inhibiting a target transcript in a mammalian subject comprising administering the composition of claim 23 to the respiratory system of a subject by introducing the composition into the vascular system of the subject.

39. The method of claim 38, wherein the solid organ is the lung.

40. The method of claim 38, wherein the composition is administered by intravenous injection.

41. The method of claim 38, wherein the composition is administered using a conventional fluid delivery technique.

42. The method of claim 38, wherein the RNAi-inducing entity comprises an siRNA.

43. The method of claim 38, wherein the RNAi-inducing entity comprises an shRNA.

44. The method of claim 38, wherein the RNAi-inducing entity comprises an RNAi-inducing vector.

45. The method of claim 38, wherein the RNAi-inducing vector comprises a DNA vector.

46. The method of claim 38, wherein the RNAi-inducing vector comprises a viral vector.

47. The method of claim 38, wherein the RNAi-inducing vector comprises a lentiviral vector.

48. The method of claim 38, wherein the RNAi-inducing vector comprises a lentivirus.

49. A method of treating or preventing a disease or clinical condition associated with overexpression or inappropriate expression of a transcript or excessive functional activity of a polypeptide encoded by the transcript comprising the step of delivering the composition of claim 23 to a solid organ or tissue of a subject at risk of or suffering from the disease or clinical condition by introducing the composition into the vascular system of the subject.

50. The composition of claim 1, wherein the delivery agent comprises a surfactant suitable for introduction into the lung.

51. The composition of claim 50, wherein the surfactant comprises 10-20% protein and 80-90% lipid by weight both based on the whole surfactant, which lipid consists of about 10% neutral lipid and of about 90% phospholipid.

52. The composition of claim 50, wherein the surfactant is derived from animal tissue or lung lavage.

53. The composition of claim 50, wherein the surfactant is synthetic.

54. The composition of claim 50, wherein the surfactant is approved by the U.S. Food and Drug Administration.

55. The composition of claim 50, wherein the surfactant is Infasurf®, Survanta®, or Exosurf®.

56. The composition of claim 50, wherein the RNAi-inducing entity comprises an siRNA

57. The composition of claim 50, wherein the RNAi-inducing entity comprises an shRNA.

58. The composition of claim 50, wherein the RNAi-inducing entity comprises an RNAi-inducing vector.

59. The composition of claim 50, wherein the RNAi-inducing entity comprises a DNA vector.

60. The composition of claim 50, wherein the RNAi-inducing entity comprises a viral vector.

61. The composition of claim 50, wherein the RNAi-inducing entity comprises a lentiviral vector.

62. The composition of claim 50, wherein the RNAi-inducing entity comprises a lentivirus.

63. A method of inhibiting a target transcript in a mammalian subject comprising administering the composition of claim 50 to the respiratory system of a subject by inhalation or intranasal delivery.

64. The method of claim 63, wherein the RNAi-inducing entity comprises an siRNA.

65. The method of claim 63, wherein the RNAi-inducing entity comprises an shRNA.

66. The method of claim 63, wherein the RNAi-inducing entity comprises an RNAi-inducing vector.

67. The method of claim 63, wherein the RNAi-inducing entity comprises a viral vector.

68. The method of claim 63, wherein the RNAi-inducing entity comprises a lentiviral vector.

69. The method of claim 63, wherein the RNAi-inducing entity comprises a lentivirus.

70. The method of claim 63, wherein the RNAi-inducing entity comprises a DNA vector.

71. A method of treating or preventing a disease or clinical condition associated with overexpression or inappropriate expression of a target transcript or excessive functional activity of a polypeptide encoded by the target transcript comprising the step of administering the composition of claim 50 to the respiratory system of a subject at risk of or suffering from the disease or clinical condition by inhalation or intranasal delivery.

72. The composition of claim 1, wherein the delivery agent is a peptide molecular transporter.

73. The composition of claim 72, wherein the peptide molecular transporter is an arginine-rich peptide containing at least 4 arginine residues.

74. The composition of claim 72, wherein the RNAi-inducing entity comprises an siRNA.

75. The composition of claim 72, wherein the RNAi-inducing entity comprises an shRNA.

76. The composition of claim 72, wherein the RNAi-inducing entity comprises an RNAi-inducing vector.

77. The composition of claim 72, wherein the RNAi-inducing entity comprises a viral vector.

78. The composition of claim 72, wherein the RNAi-inducing entity comprises a lentiviral vector.

79. The composition of claim 72, wherein the RNAi-inducing entity comprises a lentivirus.

80. The composition of claim 72, wherein the RNAi-inducing entity comprises a DNA vector.

81. A method of inhibiting expression of a target transcript in a mammalian subject comprising the step of administering to the subject a composition comprising:
(i) an RNAi-inducing entity targeted to the target transcript; and
(ii) a delivery agent selected from the group consisting of: cationic polymers, modified cationic polymers, peptide molecular transporters, surfactants suitable for introduction into the lung, lipids, liposomes, lipopolyplexes, non-cationic polymers, modified non-cationic polymers, bupivacaine, and chloroquine.

82. The method of claim 81, wherein adminstration of the composition inhibits expression of the target transcript in the lung.

83. The method of claim 81, wherein administration of the composition inhibits expression of the target transcript in at least one tissue or organ other than the lung, in addition to, or instead of, inhibiting the transcript in the lung.

84. A method of treating or preventing a disease or condition associated with overexpression or inappropriate expression of a transcript or inappropriate or excessive expression or activity of a polypeptide encoded by the transcript, the method comprising steps of:
(a) providing a subject at risk of or suffering from a disease or condition associated with overexpression or inappropriate expression of a transcript or inappropriate or excessive expression or activity of a polypeptide encoded by the transcript; and
(b) administering to the subject a composition comprising:
(i) an RNAi-inducing entity targeted to the target transcript; and
(ii) a delivery agent selected from the group consisting of: cationic polymers, modified cationic polymers, peptide molecular transporters, surfactants suitable for introduction into the lung, lipids, liposomes, non-cationic polymers, modified non-cationic polymers, bupivacaine, and chloroquine.

85. The method of claim 84, wherein the composition is administered by inhalation or intranasally.

86. The composition of claim 85, wherein the composition is administered as an aerosol.

87. The method of claim 84, wherein the composition is administered intravenously.

88. The method of claim 87, wherein the composition is administered using a conventional intravenous administration technique.

89. The method of claim 84, wherein the delivery agent comprises a delivery enhancing moiety to enhance delivery to a cell of interest.

90. The method of claim 89, wherein the delivery-enhancing moiety comprises an antibody, antibody fragment, or ligand that specifically binds to a molecule expressed by the cell of interest.

91. A composition comprising:
an analog of an siRNA or shRNA whose presence within a cell results in production of an siRNA or shRNA, wherein the siRNA or shRNA is targeted to a target transcript, wherein the analog differs from the siRNA or shRNA in that it contains at least one modification that results in increased stability, enhanced absorption, or enhanced cellular entry of the siRNA or shRNA; and
a delivery agent selected from the group consisting of: cationic polymers, modified cationic polymers, peptide molecular transporters, surfactants suitable for introduction into the lung, liposomes, non-cationic polymers, modified non-cationic polymers, bupivacaine, and chloroquine.

92. The composition of claim 91, wherein:
the modification modifies a base, a sugar, or an internucleoside linkage.

93. The composition of claim 91, wherein:
the modification is not a nucleotide 2′ modification.

94. The composition of claim 91, wherein:
the modification is a nucleotide 2′ modification.

95. The composition of claim 91, wherein:
the analog differs from the siRNA or shRNA in that at least one ribonucleotide is replaced by a deoxyribonucleotide.

96. A method of inhibiting a target transcript in a subject comprising administering the composition of claim 91 to the subject, wherein the RNAi-inducing agent is targeted to the target transcript.

97. A method of treating or preventing a disease or condition associated with overexpression or inappropriate expression of a transcript or inappropriate or excessive expression or activity of a polypeptide encoded by the transcript, the method comprising steps of:
(a) providing a subject at risk of or suffering from a disease or condition associated with overexpression or inappropriate expression of a transcript or inappropriate or excessive expression or activity of a polypeptide encoded by the transcript; and
(b) administering the composition of claim 91 to the subject, wherein the RNAi-inducing agent is targeted to the target transcript.
